(19) United States
(12) Patent Application Publication
(10) Pub. No.: US 2006/0121539 A1
(43) Pub. Date: Jun. 8, 2006

(54) Eph receptor tumor biomarkers

(75) Inventors: Waldemar Debinski, Winston-Salem, NC (US); Denise M. Gibo, Winston-Salem, NC (US); Jill E. Wykosky, Winston-Salem, NC (US)

Correspondence Address: AKERMAN SENTERFITT, P.O. BOX 3188, WEST PALM BEACH, FL, 33402-3188, US

(73) Assignee: The Penn State Research Foundation, University Park, PA (US)

(21) Appl. No.: 11/256,684

(22) Filed: Oct. 21, 2005

Related U.S. Application Data
(60) Provisional application No. 60/620,952, filed on Oct. 21, 2004.

(51) Int. Cl. G01N 33/574; C07K 14/82; C07K 16/30

(52) U.S. Cl. 435/7.23; 530/350; 530/388.22

(57) ABSTRACT

Specific, differential expression of EphA2 in both GBM cells and human GBM tumors compared to normal brain are shown. EphA2 serves as a useful molecular marker for cancer in such areas as diagnosis and prognosis. In addition, EphA2 is used in the development of new therapeutics for tumors, such as molecularly targeted drug delivery.

### PRIORITY DATA

This application claims the benefit of U.S. Provisional Application No. 60/620,952, filed Oct. 21, 2004, which is incorporated herein by reference in its entirety.

### FIELD OF THE INVENTION

The invention provides for the reliable detection and identification of biomarkers, important for the diagnosis and prognosis of cancer. The protein/peptide profile in patients with tumor cells are distinguished from normal individuals using inexpensive techniques. These techniques provide simple yet sensitive approaches to diagnosing cancer using biological fluids.

### BACKGROUND

The Eph receptors comprise the largest family of tyrosine kinase receptors, a group of transmembrane proteins that are crucial in mediating important signal transduction pathways in cells such as those controlling growth, migration, and differentiation. The fourteen members of the Eph receptors are divided into A and B classes based on the similarity of their extracellular domains and their ability to interact with their membrane-bound ligands, the ephrins. The fact that their endogenous ligands are anchored to the surface of neighboring cells makes them unique among the receptor tyrosine kinases, which typically bind soluble factors such as epidermal growth factor (EGF) and vascular endothelial growth factor (VEGF).

Most Eph receptors play an important role in axon guiding during neural development through the mediation of contact-dependent processes between cells.

Diagnosis of tumors, especially in the initial stage are difficult to diagnose and in most cases difficult to treat. Problems include: 1.) Patients are usually diagnosed 6 months to a year before death. Hence, a significant tumor mass is already established by the time treatment is initiated (2) The cancer cells invade normal tissue and do not establish defined barriers, thus surgery yields incomplete removal of residual tumor cells. (3) The brain is protected from the external environment by the blood brain barrier. This barrier is compromised in regions of the brain but there are areas that are still protected and thus accessibility to chemotherapy is restricted. The potency of standard chemotherapy is further reduced by expression of P-glycoprotein encoded by the multidrug resistance-1 (MDR) gene in capillary endothelium of brain capillaries. (4) Many primary tumors metastasize to the brain yielding multiple lesions further complicating therapy. (5) The knowledge base for brain cancer is greatly reduced in comparison to other types of cancer. Making it difficult to define molecular targets and also predicting the course of the disease. Standard therapy is surgical resection and postoperative radiation. Due to the tumor location, complete removal of the tumor may not be possible and some gliomas are completely inoperable.

Thus there is an urgent need in the art to diagnose tumor growth at the early stages and to identify therapeutic molecules that target cancer cells without affecting the surrounding normal tissue.

### SUMMARY

The present invention provides tumor biomarkers that are differentially present in the samples of patients suffering from cancer and/or cancer related disorders as compared to samples of control subjects. The present invention also provides sensitive and quick methods and kits that can be used as an aid for diagnosis of cancer and/or cancer related disorders by detecting these markers. The measurement of these markers, alone or in combination, in patient samples provides information that a diagnostician can correlate with a probable diagnosis of the extent of cancer (e.g., normal or cancer-free). Markers can be characterized by molecular weight. The markers can be resolved from other proteins in a sample by using a variety of fractionation techniques, e.g., chromatographic separation coupled with mass spectrometry, or by traditional immunoassays. In preferred embodiments, the method of resolution involves Surface-Enhanced Laser Desorption/Ionization (“SELDI”) mass spectrometry, in which the surface of the mass spectrometry probe comprises adsorbents that bind the markers.

In a preferred embodiment, various compositions are provided to further aid in the diagnosis of cancer. Preferred biomarker compositions are Eph receptors and ephrin molecules. Exemplary EPH receptors include the EphA1, EphA2, EphA3, EphA4, EphA5, EphA6, EphA7, EphA8, EphB1, EphB2, EphB3, EphB4 and EphB5, eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk and nuk receptors. Ephrin molecules include, but not limited to, ephrin-A1, ephrin-A2, ephrin-A3, ephrin-A4, ephrin-A5, ephrin-B1, ephrin-B2 and ephrin-B3. Eph receptor biomarkers include the membrane form of the receptor protein, as well as soluble extracellular fragments which retain the ability to bind their specific ligand. Preferably each of the markers in the compositions are purified.

In another preferred embodiment, the invention features an Ephrin A1 polypeptide, preferably a substantially pure preparation of an Ephrin A1 polypeptide, or a recombinant Ephrin A1 polypeptide. Ephrin A1 is a natural ligand for EphA2 and can serve as a vector for therapeutic deliveries and candidate therapeutic agent discovery. In preferred embodiments the polypeptide has a biological activity associated with its binding to an EphA2 receptor, though it may be able to either agonize or antagonize signal transduction by the EphA2 receptor. The polypeptide can be identical to the mammalian Ephrin A1 polypeptide, or it can merely be homologous to that sequence. For instance, the polypeptide preferably has an amino acid sequence at least 70% homologous to Ephrin A1, though higher sequence homologies of, for example, 80%, 85%, 90% or 95% are also contemplated. The polypeptide can comprise the full length Ephrin A1 protein, or it can comprise a fragment of that protein, which fragment may be, for instance, at least 5, 10, 20, 50 or 100 amino acids in length. The polypeptide can be glycosylated, or, by virtue of the expression system in which it is produced, or by modification of the protein sequence to preclude glycosylation, reduced carbohydrate analogs can be provided. Likewise, Ephrin A1 polypeptides can be generated which lack an endogenous signal sequence (though this is typically cleaved off even if present in the pro-form of the protein), or which lack a phosphatidylinositol linkage site to preclude addition of phosphatidylinositol. Polypeptides which lack at least the last 15 amino acid residues and polypeptides which are truncated anywhere are also contemplated.

Moreover, the polypeptide can be either an agonist (e.g. mimics), or alternatively, an antagonist of a biological activity of a naturally occurring form of the protein, e.g., the polypeptide is able to modulate growth and/or differentiation of a cell which expresses an EphA2 receptor.

In another preferred embodiment, the ephrin molecule is a targeting ligand for candidate therapeutic compounds and is used to identify candidate therapeutic compounds. In a preferred embodiment, a method of identifying candidate therapeutic agents for treatment of tumors, comprises culturing an isolated cell expressing a receptor comprising any one of: ephrin A1, ephrin-A2, ephrin-A3, ephrin-A4, ephrin-A5, ephrin-B1, ephrin-B2 or ephrin-B3, variants and fragments thereof, administering a candidate therapeutic agent to the cultured cell; and, correlating expression levels and phosphorylation of the receptor in the presence or absence of a candidate therapeutic agent as compared to a normal cell and a cell cultured in the presence of an ephrin molecule; thereby, identifying candidate therapeutic agents that bind to ephrin molecules, and identifying candidate therapeutic agents for treatment of tumors. Preferably, the candidate therapeutic agents are administered to normal cells and cancer cells and assayed for binding to ephrin molecules. Appropriate controls are used, such as for example, binding of the candidate therapeutic agents to ephrin molecules is compared to binding of said agents to normal cells and cancer cells. The effects of these candidate therapeutic agents have by binding to ephrin molecules is compared to expression and activation of Eph molecules, wherein activation of Eph molecules is determined by phosphorylation of tyrosine molecules as compared to Eph on a normal cell, Eph in the presence of its ephrin ligand and Eph in the presence of a candidate therapeutic agent. Preferably, expression of Eph is compared to Eph in a normal cell, Eph+ cells in the presence of its ephrin ligand and Eph+ cells in the presence of a candidate therapeutic agent.

In a preferred embodiment, a peptide having at least one biological activity of the Ephrin A1 polypeptide may differ in amino acid sequence, but such differences result in a modified protein which functions in the same or similar manner as a native Ephrin A1 protein or which has the same or similar characteristics of a native Ephrin A1 protein. However, homologs of the naturally occurring protein are contemplated which are antagonistic of the normal physiological role of the naturally occurring protein.

In another preferred embodiment, the invention provides variants of the polypeptide compositions described herein. Polypeptide variants generally encompassed by the present invention will typically exhibit at least about 70%, 75%, 80%, 85%, 90%, 91%, 92%, 93%, 94%, 95%, 96%, 97%, 98%, or 99% or more identity (determined as described below), along its length, to a polypeptide sequence set forth herein.

In other preferred embodiments, comparative protein profiles are generated using a microarray from patients diagnosed with cancer and from patients without known neoplastic cancers. A subset of biomarkers is selected based on collaborative results from supervised analytical methods. Examples of analytical methods include the Classification And Regression Tree (CART), implemented in for example, Biomarker Pattern Software V4.0 (BPS) (Ciphergen, CA), and the Unified Maximum Separability Analysis (UMSA) procedure, implemented in ProPeak (3Z nfomilatics, SC).

In a preferred embodiment, the analytical methods are used individually and in cross-comparison to screen for peaks that are most contributory towards the discrimination between cancer patients and the non-cancer controls. While the absolute identity of these markers is not yet known, such knowledge is not necessary to measure them in a patient sample, because they are sufficiently characterized by, e.g., mass and by affinity characteristics. It is noted that molecular weight and binding properties are characteristic properties of these markers and not limitations on means of detection or isolation. Furthermore, using the methods described herein or other methods known in the art, the absolute identity of the markers can be determined.

Preferred methods for detection and diagnosis of cancer comprise detecting at least one or more protein biomarkers in a subject sample, and; correlating the detection of one or more protein biomarkers with a diagnosis of cancer, wherein the correlation takes into account the detection of one or more biomarker in each diagnosis, as compared to normal subjects.

In other preferred embodiments, a plurality of the biomarkers are detected, preferably at least two of the biomarkers are detected, more preferably at least three of the biomarkers are detected, most preferably at least four of the biomarkers are detected.

In one aspect, the amount of each biomarker is measured in the subject sample and the ratio of the amounts between the markers is determined. Preferably, the amount of each biomarker in the subject sample and the ratio of the amounts between the biomarkers and known cancer markers is also determined to assess the stage of cancer.

In another aspect, preferably a single biomarker is used in combination with one or more known cancer biomarkers for diagnosing cancer, more preferably a plurality of the markers are used in combination with one or more known cancer markers for diagnosing cancer. Preferred cancer markers are cancer markers for diagnosing cancer, such as Eph. It is preferred that one or more protein biomarkers are used in comparing protein profiles from patients susceptible to, or suffering from cancer with normal subjects.

Preferred detection methods include use of a biochip array. Biochip arrays useful in the invention include protein and nucleic acid arrays. One or more markers are immobilized on the biochip array and subjected to laser ionization to detect the molecular weight of the markers. Analysis of the markers is, for example, by molecular weight of the one or more markers against a threshold intensity that is normalized against total ion current. Preferably, logarithmic transformation is used for reducing peak intensity ranges to limit the number of markers detected.

In another preferred method, data is generated on immobilized subject samples on a biochip array, by subjecting said biochip array to laser ionization and detecting intensity of signal for mass/charge ratio; and, transforming the data into computer readable form; and executing an algorithm that classifies the data according to user input parameters, for detecting signals that represent markers present in cancer patients and are lacking in non-cancer subject controls.

Preferably the biochip surfaces are, for example, ionic, anionic, comprised of immobilized nickel ions. comprised of a mixture of positive and negative ions, comprises one or more antibodies, single or double stranded nucleic acids, comprises proteins, peptides or fragments thereof, amino acid probes, comprises phage display libraries.

In other preferred methods one or more of the markers are detected using laser desorption/ionization mass spectrometry, comprising, providing a probe adapted for use with a mass spectrometer comprising an adsorbent attached thereto, and; contacting the subject sample with the adsorbent, and; desorbing and ionizing the marker or markers from the probe and detecting the deionized/ionized markers with the mass spectrometer.

Preferably, the laser desorption/ionization mass spectrometry comprises, providing a substrate comprising an adsorbent attached thereto; contacting the subject sample with the adsorbent; placing the substrate on a probe adapted for use with a mass spectrometer comprising an adsorbent attached thereto; and, desorbing and ionizing the marker or markers from the probe and detecting the desorbed/ionized marker or markers with the mass spectrometer.

The adsorbent can for example be, hydrophobic, hydrophilic, ionic or metal chelate adsorbent, such as, nickel or an antibody, single- or double stranded oligonucleotide, amino acid, protein, peptide or fragments thereof.

In another embodiment, a process for purification of a biomarker, comprising fractionating a sample comprising one or more protein biomarkers by size-exclusion chromatography and collecting a fraction that includes the one or more biomarker; and/or fractionating a sample comprising the one or more biomarkers by anion exchange chromatography and collecting a fraction that includes the one or more biomarkers. Fractionation is monitored for purity on normal phase and immobilized nickel arrays. Generating data on immobilized marker fractions on an array, is accomplished by subjecting said array to laser ionization and detecting intensity of signal for mass/charge ratio; and, transforming the data into computer readable form; and executing an algorithm that classifies the data according to user input parameters, for detecting signals that represent markers present in cancer patients and are lacking in non-cancer subject controls. Preferably fractions are subjected to gel electrophoresis and correlated with data generated by mass spectrometry. In one aspect, gel bands representative of potential markers are excised and subjected to enzymatic treatment and are applied to biochip arrays for peptide mapping.

In another preferred embodiment, a method of treatment for subjects with EphA2 positive tumors comprises administering a therapeutically effective amount of a pharmaceutical composition comprising ephrins, ephrin A1, peptides, variants and fragments thereof to a patient in need of treatment; and, modulating expression of Eph in tumors; thereby, ameliorating EphA2 positive tumors. Preferably, the ephrin molecules modulate expression of Eph by binding the EphA2 receptor, activating the receptor an/or cellular internalization of the EphA2 receptor.

In a preferred embodiment, modulation of Eph is by administration of a therapeutically effective amount of ephrin molecules or antibodies specific for Eph. Preferred ranges are from about 0.1 to about 300 milligrams per kilogram body weight of the vertebrate subject, more preferably, ranges from about 0.2 to about 200 milligrams per kilogram body weight of the vertebrate subject, more preferably, ranges from about 0.5 to about 20 milligrams per kilogram body weight of the vertebrate subject.

In another preferred embodiment, the active versus the inactive state of Eph receptor is measured by detecting the phosphorylation state of EphA2 molecules. Experimental details are provided in the Examples section which follows.

In another preferred embodiment, ephrin molecules are ephrin-A1, ephrin-A2, ephrin-A3, ephrin-A4, ephrin-A5, ephrin-B1, ephrin-B2 and ephrin-B3 proteins, peptides, variants, and fragments thereof.

In another preferred embodiment, the pharmaceutical compositions comprising ephrins are administered to a patient prior to, in conjunction with, or after administration of chemotherapeutic agents. Administration of pharmaceutical compositions and chemotherapeutic agents to a patient comprises intravenous administration, intrasynovial administration, transdermal administration, intramuscular administration, subcutaneous administration or oral administration.

In another preferred embodiment, the pharmaceutical composition comprises antibodies that specifically bind to EphA2.

In another preferred embodiment, a method of determining malignancy or invasiness of a tumor, comprises detecting at least one or more Eph biomarkers and/or ligands thereof, in a subject sample, and; correlating the detection of one or more Eph biomarkers and/or ligands thereof, with a diagnosis of a malignant tumor, wherein the correlation takes into account the detection of one or more biomarker in each diagnosis, as compared to normal subjects wherein the Eph biomarkers are one or more of: EphA1, EphA2, EphA3, EphA4, EphA5, EphA6, EphA7, EphA8, EphB1, EphB2, EphB3, EphB4 and EphB5, eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk and nuk receptors, peptides or fragments thereof, and ligands comprise at least one of: ephrin A1, ephrin-A2, ephrin-A3, ephrin-A4, ephrin-A5, ephrin-B1, ephrin-B2 or ephrin-B3, variants and fragments thereof, and; correlating the detection of one or more biomarkers with a diagnosis of malignancy, wherein the correlation takes into account the detection of one or more biomarkers in each diagnosis, as compared to normal subjects. Methods for determining invasiness of a tumor are detailed in the Examples which follow, such as the invasion assay.

In another preferred embodiment, the Eph biomarker is inactive EphA2 and is detected in increased levels as compared to benign tumors and normal cells.

In yet another preferred embodiment, a method of identifying candidate therapeutic agents for treatment of tumors, comprises culturing an isolated cell expressing a receptor comprising any one of: EphA1, EphA2, EphA3, EphA4, EphA5, EphA6, EphA7, EphA8, EphB1, EphB2, EphB3, EphB4 and EphB5, eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk or nuk receptors, proteins, peptides, variants, fragments and derivatives thereof; and, administering a candidate therapeutic agent to the cultured cell; correlating expression levels and phosphorylation of the receptor in the presence or absence of a candidate therapeutic agent as compared to a normal cell and a cell cultured in the presence of an ephrin molecule; thereby, identifying candidate therapeutic agents that decrease Eph expression and activate Eph molecules, thereby, identifying candidate therapeutic agents for treatment of tumors. The activation of Eph molecules is determined by phosphorylation of tyrosine molecules as compared to Eph on a normal cell, Eph in the presence of its ephrin ligand and Eph in the presence of a candidate therapeutic agent. Phosphorylation assays for determining the activation or phosphorylation state are described in the Examples which follow. The expression of Eph is s compared to Eph in a normal cell, Eph+ cells in the presence of its ephrin ligand and Eph+ cells in the presence of a candidate therapeutic agent.

In another preferred embodiment, the invention provides a composition for modulating Eph receptors comprising ephrin molecules, ephrin specific antibodies, peptides, variants and fragments thereof. Preferably, the ephrin molecules further comprise ephrin A1, ephrin-A2, ephrin-A3, ephrin-A4, ephrin-A5, ephrin-B1, ephrin-B2 or ephrin-B3, variants and fragments thereof. Binding of the ephrin molecule to Eph activates Eph and causes internalization of the receptor-ligand complex by the cell.

The invention further provides for kits for aiding the diagnosis of cancer, comprising: an adsorbent attached to a substrate, wherein the adsorbent retains one or more biomarkers.

In a preferred embodiment, a kit for diagnosing cancer in a subject, the kit comprises at least one biomarker identified by any one of EphA1, EphA2, EphA3, EphA4, EphA5, EphA6, EphA7, EphA8, EphB1, EphB2, EphB3, EphB4 and EphB5, eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk and nuk; a substrate for holding a biological sample isolated from a human subject suspected of having a damaged nerve cell, an antibody that detects at least one or more of the biomarkers; and, printed instructions for reacting the agent with the biological sample or a portion of the biological sample to detect the presence or amount of at least one marker in the biological sample. Preferably the kit comprises a plurality of biomarkers as identified by EphA1, EphA2, EphA3, EphA4, EphA5, EphA6, EphA7, EphA8, EphB1, EphB2, EphB3, EphB4 and EphB5, eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk and nuk.

In another preferred embodiment, the kit comprises at least one antibody that is specific for any one of: EphA1, EphA2, EphA3, EphA4, EphA5, EphA6, EphA7, EphA8, EphB1, EphB2, EphB3, EphB4 and EphB5, eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk and nuk.

Preferably, the kit comprises written instructions for use of the kit for detection of cancer and the instructions provide for contacting a test sample with the absorbent and detecting one or more biomarkers retained by the adsorbent.

The kit provides a substrate which allows for adsorption of said adsorbent. Preferably, the substrate can be hydrophobic, hydrophilic, charged, polar, metal ions.

The kit also provides for an adsorbent wherein the adsorbent is an antibody, single or double stranded oligonucleotide, amino acid, protein, peptide or fragments thereof.

Detection of one or more protein biomarkers using the kit, is by mass spectrometry or immunoassays such as an ELISA.

In another embodiment, various compositions are provided to further aid in the diagnosis of cancer. Preferably each of the markers in the compositions are purified.

In another prefer-red embodiment, the invention provides variants of the polypeptide compositions described herein. Polypeptide variants generally encompassed by the present invention will typically exhibit at least about 70%, 75%, 80%, 85%, 90%, 91%, 92%, 93%, 94%, 95%, 96%, 97%, 98%, or 99% or more identity (determined as described below), along its length, to a polypeptide sequence set forth herein.

In one preferred embodiment, the polypeptide fragments and variants provided by the present invention are immunologically reactive with an antibody and/or T-cell that reacts with a full-length polypeptide.

In another preferred embodiment, the polypeptide fragments and variants provided by the present invention exhibit a level of immunogenic activity of at least about 50%, preferably at least about 70%, and most preferably at least about 90% or more of that exhibited by a full-leiigth polypeptide sequence specifically set forth herein.

In other preferred embodiments, a plurality of the biomarkers are detected, preferably at least two of the biomarkers are detected, more preferably at least three of the biomarkers are detected, most preferably at least four of the biomarkers are detected.

In one aspect, the amount of each biomarker is measured in the subject sample and the ratio of the amounts between the markers is determined. Preferably, the amount of each biomarker in the subject sample and the ratio of the amounts between the biomarkers and compared to normal healthy individuals. The increase in ratio of amounts of biomarkers between healthy individuals and individuals suffering from cancer is indicative of the cancer magnitude, disorder progression as compared to clinically relevant data.

Preferably, biomarkers that are detected at different stages of cancer and clinical cancer are correlated to assess anatomical cancer, type of cellular cancer. Monitoring of which biomarkers are detected at which stage, degree of cancer will provide panels of biomarkers that provide specific information on mechanisms of cancer, identify multiple subcellular sites of cancer, identify multiple cell types involved in cancer related cancer and identify the anatomical location of cancer.

In another aspect, preferably a single biomarker is used in combination with one or more biomarkers from normal, healthy individuals for diagnosing cancer, location of cancer and progression of cancer and/or cancer related disorders, more preferably a plurality of the markers are used in combination with one or more biomarkers from normal, healthy individuals for diagnosing cancer, location of cancer and progression of cancer and/or cancer related disorders. It is preferred that one or more protein biomarkers are used in comparing protein profiles from patients susceptible to, or suffering from cancer and/or cancer related disorders, with normal subjects.

Other aspects of the invention are described infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 shows the EphA2 fluorescence in glioblastoma cells (GBM).

FIG. 2 shows results obtained with EphA2 fluorescence in snap frozen human GBM tissue sections.

FIG. 3 shows the results obtained with immunohistocytochemistry in paraffin-embedded human GBM tissue sections.

FIG. 4 is a Western blot of EphA2 immunoreactivity in GBM cells, transformed glial cells and normal brain.

FIG. 5 is a Western blot of EphA2 immunoreactivity in human GBM tumors and normal brain.

FIG. 6 shows the results obtained showing EphA2 expression on cDNA microarrays.

FIGS. 7A and 7B show EphA2 and ephrin A1 immunoreactivity in GBM cells and normal brain. FIG. 7A is a Western blot for EphA2 and ephrinA1 immunoreactivity in GBM cell lines and two normal brain samples. Five GBM cell lines (A-172 MG, DBTRG-05 MG, U-251 MG, U-87 MG, G48a) and one malignant glioma (H4) were analyzed. Normal brain samples were obtained from the frontal lobe tissue a frozen human normal brain (Normal brain I) or a normal brain protein medley (Normal brain II). FIG. 7B is a Western blot showing HUVEC non-induced and induced with 50 ng/mL TNF-α.

FIGS. 8A and 8B show EphA2 and ephrinA1 immunofluorescence in GBM cells. FIG. 5A shows the immunofluorescence for EphA2 in seven GBM cell lines (DBTRG-05 MG, 6B53-11, A-172 MG, G48a, U-251 MG, U-373 MG, and U-87 MG). FIG. 8B shows the immunofluorescence for ephrinA1 in four GBM cell lines (U-251 MG, DBTRG-05 MG, A-172 MG, and G48a). Red color represents the use of a polyclonal EphA2 antibody; green color represents the use of a monoclonal EphA2 antibody. DAPI staining reveals nuclei of cells stained for ephrinA1.

FIG. 9 shows EphA2 and ephrinA1 immunoreactivity in human GBM and normal brain tissue. Western blot of EphA2 and ephrinA1 immunoreactivity performed in GBM tumor lysates and normal brain. Normal brain was obtained from the same sources as in FIGS. 8A and 8B.

FIGS. 10A and 10B show EphA2 and ephrinA1 expression in human GBM and normal brain frozen tissue sections. FIG. 10A shows the results obtained by immunofluorescence for EphA2 and FIG. 10B shows ephrinA1 immunofluorescence in five snap-frozen human GBM tissue samples and normal brain. Nuclei were stained with DAPI.

FIGS. 11A and 11B show EphA2 and ephrinA1 expression in human GBM and normal brain paraffin-embedded tissue sections. FIG. 11A shows EphA2 expression and FIG. 11B shows ephrinA1 IHC of paraffin-embedded sections of GBM and normal brain tissue. Three representative areas of each section are shown. Arrow: specific endothelial cell staining for ephrinA1 in normal brain FIG. 11B.

FIGS. 12A and 12B are graphs showing the effect of ephrinA1 on the anchorage-independent growth of GBM cells. The ability of (FIG. 12A) Q-251 MG and DBTRG-05 MG and (FIG. 12B) H4 and U-87 MG cells to form colonies in soft agar was quantified by counting and averaging the number of cell clusters greater than or equal to 75 cells (U-251 MG, DBTRG-05 MG) or 25 cells (H4, U-87 MG) in 10 random low-power fields in each of three separate assays for each concentration of ephrinA1, 14 days after initial plating. In some fields of cells treated with 1 μg/mL of ephrinA1, there were no visible cell clusters greater than or equal to 75 or 25 cells. Vertical bars represent the standard deviation of the three separate assays.

FIGS. 13A to 13C are graphs are graphs showing the effect of ephrinA1 on the invasiveness of GBM cells. Invasiveness of (FIG. 13A) A-172-MG and (FIG. 13B) U-251-MG, and (FIG. 13C) U-87 MG GBM cells treated with various concentrations of ephrinA1. Invasion was determined as a percentage of cells migrating through Matrigel membranes as compared to control membranes. Each percentage represents the mean of 3 independent invasion chamber assays for, each concentration of ephrinA1; the vertical bars represent the standard deviation.

FIGS. 14A to 14C are Western blots showing EphA2 tyrosine phosphorylation in GBM cells. Phosphotyrosine and EphA2 detected by Western blotting following immunoprecipitation with EphA2 in (FIG. 14A) untreated GBM cell lines and tumor specimens and (FIG. 14B) U-251 MG cells treated with 1 μg/mL of ephrinA1 or IgG1 isotype control for the indicated times. FIG. 14C shows that immunoreactive EphA2 in ephrinA1-treated U-251 MG cells not subject to immunoprecipitation. A-431 cells treated with EGF were used as a positive control for the phosphotyrosine antibody (PY20), which detects phosphorylated EGF receptor at 170 kDa.

FIGS. 15A to 15C show EphrinA1 and DT390-ephrinA1 recombinant proteins expression in E. coli and partial purification of DT390-ephrinA1 cytotoxin. FIG. 15A is a gel and a Western blot of the gel. Ephrin A1 is a major bacterial protein upon induction with IPTG. 1, molecular size markers; 2, pre-induced lysated of cells; 3, IPTG-induced production of ephrin At in bacteria (lanes 1 to 3 represent SDS-PAGE); 4, pre-induced lysated of cells; 5, IPTG-induced production of ephrin A1in bacteria (lanes 4 to 5 represent Western blot). FIG. 15B is a gel and a Western blot of the gel. DT390-ephrin A1 is a major bacterial protein upon induction with IPTG. I, molecular size markers; II, pre-induced lysate of cells; III, IPTG-induced production of DT390-ephrin A1 in bacteria (lanes I to III represent SDS-PAGE); IV, Western blot using anti-ephrinA1 antibody of partially purified DT390-ephrinA1 cytotoxin. FIG. 15C is a graph showing the results of a cell proliferation assay in U-215 MG GBM cells using DT390-ephrinA1 (15 nM) in the absence or presence of an excess of ephrinA1-Fc.

### DETAILED DESCRIPTION

The present invention identifies biomarkers that are diagnostic of cancer and/or cancer related disorders. Detection of different biomarkers of the invention are also diagnostic of the degree of severity of cancer, the cell(s) involved, and prognosis of the disease. In particular, the invention employs a step of correlating the presence or amount of one or more Eph receptor protein(s) with the severity and/or type of cancer. The amount of a biomarker, fragment or derivative thereof directly relates to prognosis, diagnosis, tumor progression, and particular stage of cancer.

### DEFINITIONS

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein.

As used herein, the terms “Eph receptor” or “Eph-type receptor” refer to a class of receptor tyrosine kinases, comprising at least eleven paralogous genes, though many more orthologs exist within this class, e.g. homologs from different species. Eph receptors, in general, are a discrete group of receptors related by homology. They are characterized by an extracellular domain containing a characteristic spacing of cysteine residues near the N-terminus and two fibronectin type III repeats. Exemplary Eph receptors include the EphA1, EphA2, EphA3, EphA4, EphA5, EphA6, EphA7, EphA8, EphB1, EphB2, EphB3, EphB4 and EphB5, eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk and nuk receptors. The term “EPH receptor” refers to the membrane form of the receptor protein, as well as soluble extracellular fragments which retain the ability to bind their specific ligand. Ligands include, but not limited to, ephrin-A1, ephrin-A2, ephrin-A3, ephrin-A4, ephrin-A5, ephrin-B1, ephrin-B2 and ephrin-B3. Furthermore, “mek4/sek type receptors” refers to a closely related subgroup of the EPH receptor family, which subgroup includes: the “mek4-related receptors” such as mek4, cek4, hek and tyro4; the “sek-related receptors” such as sek, cek8, pagliaccio, tyro1 and rtk1; as well as other phylogenetically related homologs such as eek, bsk, ehk1, ehk2, and cek7. The term “ortholog” refers to genes or proteins which are homologs via speciation, e.g., closely related and assumed to have common descent based on structural and functional considerations. Orthologous proteins function as recognizably the same activity in different species. The term “paralog” refers to genes or proteins which are homologs via gene duplication, e.g., duplicated variants of a gene within a genome.

“Inactive Eph” refers to the Eph receptor in its inactive state, e.g. in the non-tyrosine-phosphorylated state. Determining the state of phosphorylation of a molecule is known in the art. Detailed methods for determining the Eph receptor phosphorylation are provided in the Examples section which follows.

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

As used herein, a “pharmaceutically acceptable” component is one that is suitable for use with humans and/or animals without undue adverse side effects (such as toxicity, irritation, and allergic response) commensurate with a reasonable benefit/risk ratio.

The term “compound” as used herein (e.g., as in “candidate therapeutic agent” or “test compound”) is meant to include both exogenously added test compounds and peptides endogenously expressed from a peptide library. For example, in certain embodiments, the reagent cell also produces the test compound which is being screened. For instance, the reagent cell can produce. e.g., a test polypeptide, a test nucleic acid and/or a test carbohydrate which is screened for its ability to modulate the receptor/channel activity. In such embodiments, a culture of such reagent cells will collectively provide a library of potential effector molecules and those members of the library which either agonize or antagonize the receptor or ion channel function can be selected and identified. Moreover, it will be apparent that the reagent cell can be used to detect agents which transduce a signal via the receptor or channel of interest.

A “chemotherapeutic agent” is a chemical compound useful in the treatment of cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenetlhiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carnomycin, carzinophilin, chromomycins, dactinomycin, daunorubicin, detorubicin, 6-diazo-5-oxo-L-norleucine, doxorubicin, epirubicin, esorubicin, idaribicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrenie; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Rhône-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantronie; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

By the term “modulate,” it is meant that any of the mentioned activities, are, e.g., increased, enhanced, increased, agonized (acts as an agonist), promoted, decreased, reduced, suppressed blocked, or antagonized (acts as an agonist). Modulation can increase activity more than 1-fold, 2-fold, 3-fold, 5-fold, 10-fold, 100-fold, etc., over baseline values. Modulation can also decrease its activity below baseline values.

The terms “patient” or “individual” are used interchangeably herein, and is meant a mammalian subject to be treated, with human patients being preferred. In some cases, the methods of the invention find use in experimental animals, in veterinary application, and in the development of animal models for cancer, including, but not limited to, rodents including mice, rats, and hamsters; and primates.

“Diagnostic” or “diagnosed” means identifying the presence or nature of a pathologic condition. Diagnostic methods differ in their sensitivity and specificity. The “sensitivity” of a diagnostic assay is the percentage of cancer related disorders individuals who test positive (percent of “true positives”). Cancer related disorders individuals not detected by the assay are “false negatives.” Subjects who are not cancer related disorders and who test negative in the assay, are termed “true negatives.” The “specificity” of a diagnostic assay is 1 minus the false positive rate, where the “false positive” rate is defined as the proportion of those without the cancer who test positive. While a particular diagnostic method may not provide a definitive diagnosis of a condition, it suffices if the method provides a positive indication that aids in diagnosis.

As used herein, “ameliorated” or “treatment” refers to a symptom which is approaches a normalized value, e.g., is less than 50% different from a normalized value, preferably is less than about 25% different from a nonmalized value, more preferably, is less than 10% different from a normalized value, and still more preferably, is not significantly different from a normalized value as determined using routine statistical tests.

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy. As used herein, “ameliorated” or “treatment” refers to a symptom which is approaches a normalized value (for example a value obtained in a healthy patient or individual), e.g., is less than 50% different from a normalized value, preferably is less than about 25% different from a normalized value, more preferably, is less than 10% different from a normalized value, and still more preferably,.is not significantly different from a normalized value as determined using routine statistical tests.

The “treatment of neoplastic cancer or neoplastic cells”, refers to a candidate therapeutic agent capable of invoking one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

As used herein, “cancer” refers to all types of cancer or neoplasm or malignant tumors found in mammals, including, but not limited to: leukemias, lymphomas, melanomas, carcinomas and sarcomas. Examples of cancers are cancer of the brain, breast, pancreas, cervix, colon, head and neck, kidney, lung, non-small cell lung, melanoma, mesothelioma, ovary, sarcoma, stomach, uterus and Medulloblastoma.

The term “leukemia” refers broadly to progressive, malignant cancers of the blood-forming organs and is generally characterized by a distorted proliferation and development of leukocytes and their precursors in the blood and bone marrow. Leukemia is generally clinically classified on the basis of (1) the duration and character of the cancer-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic). Accordingly, the present invention includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Examples of sarcomas include, but not limited to a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas include but not limited to, for example, acrallentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Carcinomas include but not limited to, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

Additional cancers include, for example, Hodgkin's Cancer, Non-Hodgkin's Lymphoma, multiple myeloma, neuroblastoma, breast cancer, cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

As used herein, “cell surface receptor” refers to molecules that occur on the surface of cells, interact with the extracellular environment, and transmit or transduce the information regarding the environment intracellularly in a manner that ultimately modulates transcription of specific promoters, resulting in transcription of specific genes.

An “allele” or “variant” is an alternative form of a gene. Of particular utility in the invention are variants of the gene encoding Eph2. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The terms “amino acid” or “amino acid sequence” refer to an oligopeptide, peptide, polypeptide, or protein sequence, or a fragment of any of these, and to naturally occurring or synthetic molecules. In this context, “fragments,” refer to fragments of Eph which are preferably at least or 10 to about 30 or 50, 60, 70, 80 90 or 100 amino acids in length, more preferably at least 15, 20, 25, 30, 40, or 50 amino acids. Where “amino acid sequence” is recited to refer to an amino acid sequence of a naturally occurring protein molecule, “amino acid sequence” and like terms are not meant to limit the amino acid sequence to the complete native amino acid sequence associated with the recited protein molecule.

As referred to herein, “fragments of a nucleic acid sequence” comprise at least about 10 or 15 nucleic acid residues (nucleotides), more preferably at least about 20, 30, 40, 50, 60, 70, 80, 90, 100, 150 or 200 nucleic acid residues.

As used herein, “extracellular signals” include a molecule or a change in the environment that is transduced intracellularly via cell surface proteins that interact, directly or indirectly, with the signal. An extracellular signal or effector molecule includes any compound or substance that in some manner specifically alters the activity of a cell surface protein. Examples of such signals include, but are not limited to, molecules such as acetylcholine, growth factors and hormones, that bind to cell surface and/or intracellular receptors and ion channels and modulate the activity of such receptors and channels.

As used herein, “extracellular signals” also include as yet unidentified substances that modulate the activity of a cellular receptor, and thereby influence intracellular functions. Such extracellular signals are potential pharmacological agents that may be used to treat specific cancers by modulating the activity of specific cell surface receptors.

“Orphan receptors” is a designation given to a receptors for which no specific natural ligand has been described.

A “test amount” of a marker refers to an amount of a marker present in a sample being tested. A test amount can be either in absolute amount (e.g., μg/ml) or a relative amount (e.g., relative intensity of signals).

A “diagnostic amount” of a marker refers to an amount of a marker in a subject's sample that is consistent with a diagnosis of cancer and/or cancer related disorder. A diagnostic amount can be either in absolute amount (e.g., μg/ml) or a relative amount (e.g., relative intensity of signals).

A “control amount” of a marker can be any amount or a range of amount which is to be compared against a test amount of a marker. For example, a control amount of a marker can be the amount of a marker in a person without cancer. A control amount can be either in absolute amount (e.g., μg/ml) or a relative amount (e.g., relative intensity of signals).

“Probe” refers to a device that is removably insertable into a gas phase ion spectrometer and comprises a substrate having a surface for presenting a marker for detection. A probe can comprise a single substrate or a plurality of substrates.

“Substrate” or “probe substrate” refers to a solid phase onto which an adsorbent can be provided (e.g., by attachment, deposition, etc.).

“Adsorbent” refers to any material capable of adsorbing a marker. The term “adsorbent” is used herein to refer both to a single material (“monoplex adsorbent”) (e.g., a compound or functional group) to which the marker is exposed, and to a plurality of different materials (“multiplex adsorbent”) to which the marker is exposed. The adsorbent materials in a multiplex adsorbent are referred to as “adsorbent species.” For example, an addressable location on a probe substrate can comprise a multiplex adsorbent characterized by many different adsorbent species (e.g., anion exchange materials, metal chelators, or antibodies), having different binding characteristics. Substrate material itself can also contribute to adsorbing a marker and may be considered part of an “adsorbent.”

“Adsorption” or “retention” refers to the detectable binding between an absorbent and a marker either before or after washing with an eluant (selectivity threshold modifier) or a washing solution.

“Eluant” or “washing solution” refers to an agent that can be used to mediate adsorption of a marker to an adsorbent. Eluants and washing solutions are also referred to as “selectivity threshold modifiers.” Eluants and washing solutions can be used to wash and remove unbound materials from the probe substrate surface.

“Resolve,” “resolution,” or “resolution of marker” refers to the detection of at least one marker in a sample. Resolution includes the detection of a plurality of markers in a sample by separation and subsequent differential detection. Resolution does not require the complete separation of one or more markers from all other biomolecules in a mixture. Rather, any separation that allows the distinction between at least one marker and other biomolecules suffices.

“Gas phase ion spectrometer” refers to an apparatus that measures a parameter which can be translated into mass-to-charge ratios of ions formed when a sample is volatilized and ionized. Generally ions of interest bear a single charge, and mass-to-charge ratios are often simply referred to as mass. Gas phase ion spectrometers include, for example, mass spectrometers, ion mobility spectrometers, and total ion current measuring devices.

“Mass spectrometer” refers to a gas phase ion spectrometer that includes an inlet system, an ionization source, an ion optic assembly, a mass analyzer, and a detector.

“Laser desorption mass spectrometer” refers to a mass spectrometer which uses laser as means to desorb, volatilize, and ionize an analyte.

“Detect” refers to identifying the presence, absence or amount of the object to be detected.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

“Detectable moiety” or a “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavidin, dioxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, or flow cytometry.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., F_{ab}′ and F_{(ab)′2} fragments. The term “antibody,” as used herein, also includes antibody fragments either produced by the. modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

“Immunoassay” is an assay that uses an antibody to specifically bind an antigen (e.g., a marker). The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Energy absorbing molecule” or “EAM” refers to a molecule that absorbs energy from an ionization source in a mass spectrometer thereby aiding desorption of analyte, such as a marker, from a probe surface. Depending on the size and nature of the analyte, the energy absorbing molecule can be optionally used. Energy absorbing molecules used in MALDI are frequently referred to as “matrix.” Cinnamic acid derivatives, sinapinic acid (“SPA”), cyano hydroxy cinnamic acid (“CHCA”) and dihydroxybenzoic acid are frequently used as energy absorbing molecules in laser desorption of bioorganic molecules.

“Substantially purified” refers to nucleic acid molecules or proteins that are removed from their natural environment and are isolated or separated, and are at least about 60% free, preferably about 75% free, and most preferably about 90% free, from other components with which they are naturally associated.

“Substrate” refers to any rigid or semi-rigid support to which nucleic acid molecules or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

Eph and Ephrin Biomarkers

The Eph receptors comprise the largest family of tyrosine kinase receptors, a group of transmembrane proteins that are crucial in mediating important signal transduction pathways in cells such as those controlling growth, migration, and differentiation. The fourteen members of the Eph receptors are divided into A and B classes based on the similarity of their extracellular domains and their ability to interact with their membrane-bound ligands, the ephrins. Endogenous ligands of Eph receptors are anchored to the surface of neighboring cells makes them unique among the receptor tyrosine kinases, which typically bind soluble factors such as epidermal growth factor (EGF) and vascular endothelial growth factor (VEGF).

Most Eph receptors play an important role in axon guiding during cancer development through the mediation of contact-dependent processes between cells. EphA2, however, is normally expressed at low levels on the surface of adult epithelial cells. It is localized to intercellular junctions, where virtually all of the receptor is bound by its ligand, ephrinA1. EphA2 is significantly overexpressed in several human epithelial cancers, including colon, breast, and pancreatic carcinoma. The unstable cell-cell contacts in cancer tissue hinder the ability of EphA2 to interact with ephrinA1 on neighboring cells. As a result, receptor activation as well as ephrinA1-induced EphA2 degradation are markedly decreased. Interestingly, this produces a situation in malignant cells in which EphA2 is significantly less activated, and at the same time, highly overexpressed. Many of the invasive and aggressive phenotypes of cancel are directly correlated with the overexpression of EphA2, both in vitro and in vivo.

In our previous analysis of cDNA microarrays, we found EphA2 to be one of the most uniformly overexpressed genes in glioblastoma multiforme (GBM), the most invasive and lethal brain tumor of astroglial origin. This finding was unexpected considering all reports of EphA2 association with cancer thus far are in malignancies of epithelial origin. If overexpressed specifically at the protein level in GBM compared to normal tissue, EphA2 is a new molecular marker and therapeutic target. Exemplary EPH receptors include the EphA1, EphA2, EphA3, EphA4, EphA5, EphA6, EphA7, EphA8, EphB1, EphB2, EphB3, EphB4 and EphB4, eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk and nuk receptors. Ligands include, but not limited to, ephrin-A1, ephrin-A2, ephrin-A3, ephrin-A4, ephrin-A5, ephrin-B1, ephrin-B2 and ephrin-B3.

In another preferred embodiment, ephrin molecules comprising any one of: ephrin A1, ephrin-A2, ephrin-A3, ephrin-A4, ephrin-A5, ephrin-B1, ephrin-B2 or ephrin-B3, variants and fragments thereof, are biomarkers of cancer.

In comparison to currently existing products, the invention provides several superior advantages and benefits. First, the identification of tumor biomarkers provide more rapid and less expensive diagnosis of cancer severity than existing diagnostic devices such as computed tomography (CT) and magnetic resonance imaging (MRI). The invention also allows quantitative detection and high content assessment of damage to organs. The invention also allows identification of the specific cell type affected (for example, neurons versus glia). In addition, levels of these tumor-specific and tumor-enriched proteins provides more accurate information regarding the diagnosis, prognosis and stage of cancer than what is available on the market.

In another preferred embodiment, diagnosis of cancer in a subject is analyzed by (a) providing a biological sample isolated from a subject suspected of having a tumor; (b) detecting in the sample the presence or amount of at least one marker selected from one or more tumor markers; and (c) correlating the presence or amount of the marker with the presence or type of tumor in the subject. Preferably, tumor cells are from any organ, tissue etc in the body. Examples of tumor cells that could express Eph receptor biomarkers are such cells that reside in the central and peripheral nerve systems, including nerve cells, glial cell, oligodendrocyte, microglia cells or tumor stem cells) in in vitro culture or in situ in an animal subjects (“tumor specific or tumor enriched” proteins); other types of cells include cardiomyocytes, myocytes in skeletal muscles, hepatocytes, kidney cells, ovarian cells, prostate, cells in testis. In some embodiments, the samples preferably comprise tumor cells, for example, a biopsy of a central nervous system or peripheral nervous system tissue are suitable biological samples for use in the invention. In addition, cellular damage can compromise the cell membrane leading to the efflux of these tumor proteins first into the extracellular fluid or space and to the cerebrospinal fluid and eventually in the circulating blood (as assisted by the compromised blood brain barrier) and other biofluids (e.g. urine, sweat, s saliva, etc.). Thus, other suitable biological samples include, but not limited to such cells or fluid secreted from these cells. Obtaining biological fluids such as cerebrospinal fluid, blood, plasma, serum, saliva and urine, from a subject is typically much less invasive and traumatizing than obtaining a solid tissue biopsy sample. Thus, samples, which are biological fluids, are preferred for use in the invention. CSF, in particular, is preferred for detecting tumor damage in a subject as it is in immediate contact with the nervous system and is readily obtainable.

A biological sample can be obtained from a subject by conventional techniques. For example, CSF can be obtained by lumbar puncture. Blood can be obtained by venipuncture, while plasma and serum can be obtained by fractionating whole blood according to known methods. Surgical techniques for obtaining solid tissue samples are well known in the art. For example, methods for obtaining a nervous system tissue sample are described in standard neurosurgery texts such as Atlas of Neurosurgery: Basic Approaches to Cranial and Vascular Procedures, by F. Meyer, Churchill Livingstone, 1999; Stereotactic and Image Directed Surgery of Brain Tumors, 1st ed., by David G. T. Thomas, WB Saunders Co., 1993; and Cranial Microsurgery: Approaches and Techniques, by L. N. Sekhar and E. De Oliveira, 1st ed., Thieme Medical Publishing, 1999. Methods for obtaining and analyzing brain tissue are also described in Belay et al., Arch. Neurol. 58: 1673-1678 (2001); and Seijo et al., J. Clin. Microbiol. 38: 3892-3895 (2000).

Any animal can be used as a subject from which a biological sample is obtained. Preferably, the subject is a mammal, such as for example, a human, dog, cat, horse, cow, pig, sheep, goat, chicken, primate, rat, or mouse. More preferably, the subject is a human. Particularly preferred are cancer patients.

The biomarkers of the invention can be detected in a sample by any means. Methods for detecting the biomarkers are described in detail in the materials and methods and Examples which follow. For example, immunoassays, include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, fluorescent immunoassays and the like. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding an antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen (i.e. tumor biomarker), coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

Identification of New Markers

In a preferred embodiment, a biological sample is obtained from a patient with a tumor. Biological samples comprising biomarkers from other patients and control subjects (i.e. normal healthy individuals of similar age, sex, physical condition) are used as comparisons. Biological samples are extracted as discussed above. Preferably, the sample is prepared prior to detection of biomarkers. Typically, preparation involves fractionation of the sample and collection of fractions determined to contain the biomarkers. Methods of pre-fractionation include, for example, size exclusion chromatography, ion exchange chromatography, heparin chromatography, affinity chromatography, sequential extraction, gel electrophoresis and liquid chromatography. The analytes also may be modified prior to detection. These methods are useful to simplify the sample for further analysis. For example, it can be useful to remove high abundance proteins, such as albumin, from blood before analysis.

In one embodiment, a sample can be pre-fractionated according to size of proteins in a sample using size exclusion chromatography. For a biological sample wherein the amount of sample available is small, preferably a size selection spin column is used. In general, the first fraction that is eluted from the column (“fraction 1”) has the highest percentage of high molecular weight proteins; fraction 2 has a lower percentage of high molecular weight proteins; fraction 3 has even a lower percentage of high molecular weight proteins; fraction 4 has the lowest amount of large proteins; and so on. Each fraction can then be analyzed by immunoassays, gas phase ion spectrometry, and the like, for the detection of markers.

In another embodiment, a sample can be pre-fractionated by anion exchange chromatography. Anion exchange chromatography allows pre-fractionation of the proteins in a sample roughly according to their charge characteristics. For example, a Q anion-exchange resin can be used (e.g., Q HyperD F, Biosepra), and a sample can be sequentially eluted with eluants having different pH's. Anion exchange chromatography allows separation of biomarkers in a sample that are more negatively charged from other types of biomarkers. Proteins that are eluted with an eluant having a high pH is likely to be weakly negatively charged, and a fraction that is eluted with an eluant having a low pH is likely to be strongly negatively charged. Thus, in addition to reducing complexity of a sample, anion exchange chromatography separates proteins according to their binding characteristics.

In yet another embodiment, a sample can be pre-fractionated by heparin chromatography. Heparin chromatography allows pre-fractionation of the markers in a sample also on the basis of affinity interaction with heparin and charge characteristics. Heparin, a sulfated mucopolysaccharide, will bind markers with positively charged moieties and a sample can be sequentially eluted with eluants having different pH's or salt concentrations. Markers eluted with an eluant having a low pH are more likely to be weakly positively charged. Markers eluted with an eluant having a high pH are more likely to be strongly positively charged. Thus, heparin chromatography also reduces the complexity of a sample and separates markers according to their binding characteristics.

In yet another embodiment, a sample can be pre-fractionated by isolating proteins that have a specific characteristic, e.g. are glycosylated. For example, a CSF sample can be fractionated by passing the sample over a lectin chromatography column (which has a high affinity for sugars). Glycosylated proteins will bind to the lectin column and non-glycosylated proteins will pass through the flow through. Glycosylated proteins are then eluted from the lectin column with an eluant containing a sugar, e.g., N-acetyl-glucosamine and are available for further analysis.

Thus there are many ways to reduce the complexity of a sample based on the binding properties of the proteins in the sample, or the characteristics of the proteins in the sample.

In yet another embodiment, a sample can be fractionated using a sequential extraction protocol. In sequential extraction, a sample is exposed to a series of adsorbents to extract different types of biomarkers from a sample. For example, a sample is applied to a first adsorbent to extract certain proteins, and an eluant containing non-adsorbent proteins (i.e., proteins that did not bind to the first adsorbent) is collected. Then, the fraction is exposed to a second adsorbent. This further extracts various proteins from the fraction. This second fraction is then exposed to a third adsorbent, and so on.

Any suitable materials and methods can be used to perform sequential extraction of a sample. For example, a series of spin columns comprising different adsorbents can be used. In another example, a multi-well comprising different adsorbents at its bottom can be used. In another example, sequential extraction can be performed on a probe adapted for use in a gas phase ion spectrometer, wherein the probe surface comprises adsorbents for binding biomarkers. In this embodiment, the sample is applied to a first adsorbent on the probe, which is subsequently washed with an eluant. Markers that do not bind to the first adsorbent are removed with an eluant. The markers that are in the fraction can be applied to a second adsorbent on the probe, and so forth. The advantage of performing sequential extraction on a gas phase ion spectrometer probe is that markers that bind to various adsorbents at every stage of the sequential extraction protocol can be analyzed directly using a gas phase ion spectrometer.

In yet another embodiment, biomarkers in a sample can be separated by high-resolution electrophoresis, e.g., one or two-dimensional gel electrophoresis. A fraction containing a marker can be isolated and further analyzed by gas phase ion spectrometry. Preferably, two-dimensional gel electrophoresis is used to generate two-dimensional array of spots of biomarkers, including one or more markers. See, e.g., Jungblut and Thiede, Mass Spectr. Rev. 16:145-162 (1997).

The two-dimensional gel electrophoresis can be performed using methods known in the art. See, e.g., Deutscher ed., Methods In Enzymology vol. 182. Typically, biomarkers in a sample are separated by, e.g., isoelectric focusing, during which biomarkers in a sample are separated in a pH gradient until they reach a spot where their net charge is zero (i.e., isoelectric point). This first separation step results in one-dimensional array of biomarkers. The biomarkers in one dimensional array is further separated using a technique generally distinct from that used in the first separation step. For example, in the second dimension, biomarkers separated by isoelectric focusing are further separated using a polyacrylamide gel, such as polyacrylamide gel electrophoresis in the presence of sodium dodecyl sulfate (SDS-PAGE). SDS-PAGE gel allows further separation based on molecular mass of biomarkers. Typically, two-dimensional gel electrophoresis can separate chemically different biomarkers in the molecular mass range from 1000-200,000 Da within complex mixtures.

Biomarkers in the two-dimensional array can be detected using any suitable methods known in the art. For example, biomarkers in a gel can be labeled or stained (e.g., Coomassie Blue or silver staining). If gel electrophoresis generates spots that correspond to the molecular weight of one or more markers of the invention, the spot can be further analyzed by densitometric analysis or gas phase ion spectrometry. For example, spots can be excised from the gel and analyzed by gas phase ion spectrometry. Alternatively, the gel containing biomarkers can be transferred to an inert membrane by applying an electric field. Then a spot on the membrane that approximately corresponds to the molecular weight of a marker can be analyzed by gas phase ion spectrometry. In gas phase ion spectrometry, the spots can be analyzed using any suitable techniques, such as MALDI or SELDI.

Prior to gas phase ion spectrometry analysis, it may be desirable to cleave biomarkers in the spot into smaller fragments using cleaving reagents, such as proteases (e.g., trypsin). The digestion of biomarkers into small fragments provides a mass fingerprint of the biomarkers in the spot, which can be used to determine the identity of markers if desired.

In yet another embodiment, high performance liquid chromatography (HPLC) can be used to separate a mixture of biomarkers in a sample based on their different physical properties, such as polarity, charge and size. HPLC instruments typically consist of a reservoir of mobile phase, a pump, an injector, a separation column, and a detector. Biomarkers in a sample are separated by injecting an aliquot of the sample onto the column. Different biomarkers in the mixture pass through the column at different rates due to differences in their partitioning behavior between the mobile liquid phase and the stationary phase. A fraction that corresponds to the molecular weight and/or physical properties of one or more markers can be collected. The fraction can then be analyzed by gas phase ion spectrometry to detect markers.

Optionally, a marker can be modified before analysis to improve its resolution or to determine its identity. For example, the markers may be subject to proteolytic digestion before analysis. Any protease can be used. Proteases, such as trypsin, that are likely to cleave the markers into a discrete number of fragments are particularly useful. The fragments that result from digestion function as a fingerprint for the markers, thereby enabling their detection indirectly. This is particularly useful where there are markers with similar molecular masses that might be confused for the marker in question. Also, proteolytic fragmentation is useful for high molecular weight markers because smaller markers are more easily resolved by mass spectrometry. In another example, biomarkers can be modified to improve detection resolution. For instance, neuraminidase can be used to remove terminal sialic acid residues from glycoproteins to improve binding to an anionic adsorbent and to improve detection resolution. In another example, the markers can be modified by the attachment of a tag of particular molecular weight that specifically bind to molecular markers, further distinguishing them. Optionally, after detecting such modified markers, the identity of the markers can be further determined by matching the physical and chemical characteristics of the modified markers in a protein database (e.g., SwissProt).

After preparation, biomarkers in a sample are typically captured on a substrate for detection. Traditional substrates include antibody-coated 96-well plates or nitrocellulose membranes that are subsequently probed for the presence of proteins. Preferably, the biomarkers are identified using immunoassays as described above. However, preferred methods also include the use of biochips. Preferably the biochips are protein biochips for capture and detection of proteins. Many protein biochips are described in the art. These include, for example, protein biochips produced by Packard BioScience Company (Meriden Conn.), Zyomyx (Hayward, Calif.) and Phylos (Lexington, Mass.). In general, protein biochips comprise a substrate having a surface. A capture reagent or adsorbent is attached to the surface of the substrate. Frequently, the surface comprises a plurality of addressable locations, each of which location has the capture reagent bound there. The capture reagent can be a biological molecule, such as a polypeptide or a nucleic acid, which captures other biomarkers in a specific manner. Alternatively, the capture reagent can be a chromatographic material, such as an anion exchange material or a hydrophilic material. Examples of such protein biochips are described in the following patents or patent applications: U.S. Pat. No. 6,225,047 (Hutchens and Yip, “Use of retentate chromatography to generate difference maps,” May 1, 2001), International publication WO 99/51773 (Kuimelis and Wagner, “Addressable protein arrays,” Oct. 14, 1999), International publication WO 00/04389 (Wagner et al., “Arrays of protein-capture agents and methods of use thereof,” Jul. 27, 2000), International publication WO 00/56934 (Englert et al., “Continuous porous matrix arrays,” Sep. 28, 2000).

In general, a sample containing the biomarkers is placed on the active surface of a biochip for a sufficient time to allow binding. Then, unbound molecules are washed from the surface using a suitable eluant. In general, the more stringent the eluant, the more tightly the proteins must be bound to be retained after the wash. The retained protein biomarkers now can be detected by appropriate means.

Analytes captured on the surface of a protein biochip can be detected by any method known in the art. This includes, for example, mass spectrometry, fluorescence, surface plasmon resonance, ellipsometry and atomic force microscopy. Mass spectrometry, and particularly SELDI mass spectrometry, is a particularly useful method for detection of the biomarkers of this invention.

Preferably, a laser desorption time-of-flight mass spectrometer is used in embodiments of the invention. In laser desorption mass spectrometry, a substrate or a probe comprising markers is introduced into an inlet system. The markers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Matrix-assisted laser desorption/ionization mass spectrometry, or MALDI-MS, is a method of mass spectrometry that involves the use of an energy absorbing molecule, frequently called a matrix, for desorbing proteins intact from a probe surface. MALDI is described, for example, in U.S. Pat. No. 5,118,937 (Hillenkamp et al.) and U.S. Pat. No. 5,045,694 (Beavis and Chait). In MALDI-MS the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenizoic acid. Other suitable energy absorbing molecules are known to those skilled in this art. The matrix dries, forming crystals that encapsulate the analyte molecules. Then the analyte molecules are detected by laser desorption/ionization mass spectrometry. MALDI-MS is useful for detecting the biomarkers of this invention if the complexity of a sample has been substantially reduced using the preparation methods described above.

Surface-enhanced laser desorption/ionization mass spectrometry, or SELDI-MS represents an improvement over MALDI for the fractionation and detection of biomolecules, such as proteins, in complex mixtures. SELDI is a method of mass spectrometry in which biomolecules, such as proteins, are captured on the surface of a protein biochip using capture reagents that are bound there. Typically, non-bound molecules are washed from the probe surface before interrogation. SELDI is described, for example, in: U.S. Pat. No. 5,719,060 (“Method and Apparatus for Desorption and Ionization of Analytes,” Hutchens and Yip, Feb. 17, 1998,) U.S. Pat. No. 6,225,047 (“Use of Retentate Chromatography to Generate Difference Maps,” Hutchens and Yip, May 1, 2001) and Weinberger et al., “Time-of-flight mass spectrometry,” in Encyclopedia of Analytical Chemistry, R. A. Meyers, ed., pp 11915-11918 John Wiley & Sons Chichesher, 2000.

Markers on the substrate surface can be desorbed and ionized using gas phase ion spectrometry. Any suitable gas phase ion spectrometers can be used as long as it allows markers on the substrate to be resolved. Preferably, gas phase ion spectrometers allow quantitation of markers.

In one embodiment, a gas phase ion spectrometer is a mass spectrometer. In a typical mass spectrometer, a substrate or a probe comprising markers on its surface is introduced into an inlet system of the mass spectrometer. The markers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of markers or other substances will typically involve detection of signal intensity. This, in turn, can reflect the quantity and character of markers bound to the substrate. Any of the components of a mass spectrometer (e.g., a desorption source, a mass analyzer, a detector, etc.) can be combined with other suitable components described herein or others known in the art in embodiments of the invention.

In another embodiment, an immunoassay can be used to detect and analyze markers in a sample. This method comprises: (a) providing an antibody that specifically binds to a marker; (b) contacting a sample with the antibody; and (c) detecting the presence of a complex of the antibody bound to the marker in the sample.

To prepare an antibody that specifically binds to a marker, purified markers or their nucleic acid sequences can be used. Nucleic acid and amino acid sequences for markers can be obtained by further characterization of these markers. For example, each marker can be peptide mapped with a number of enzymes (e.g., trypsin, V8 protease, etc.). The molecular weights of digestion fragments from each marker can be used to search the databases, such as SwissProt database, for sequences that will match the molecular weights of digestion fragments generated by various enzymes. Using this method, the nucleic acid and amino acid sequences of other markers can be identified if these markers are known proteins in the databases.

Alternatively, the proteins can be sequenced using protein ladder sequencing. Protein ladders can be generated by, for example, fragmenting the molecules and subjecting fragments to enzymatic digestion or other methods that sequentially remove a single amino acid from the end of the fragment. Methods of preparing protein ladders are described, for example, in International Publication WO 93/24834 (Chait et al.) and U.S. Pat. No. 5,792,664 (Chait et al.). The ladder is then analyzed by mass spectrometry. The difference in the masses of the ladder fragments identify the amino acid removed from the end of the molecule.

If the markers are not known proteins in the databases, nucleic acid and amino acid sequences can be determined with knowledge of even a portion of the amino acid sequence of the marker. For example, degenerate probes can be made based on the N-terminal amino acid sequence of the marker. These probes can then be used to screen a genomic or cDNA library created from a sample from which a marker was initially detected. The positive clones can be identified, amplified, and their recombinant DNA sequences can be subcloned using techniques which are well known. See, e.g., Current Protocols for Molecular Biology (Ausubel et al., Green Publishing Assoc. and Wiley-Interscience 1989) and Molecular Cloning: A Laboratory Manual, 3rd Ed. (Sambrook et al., Cold Spring Harbor Laboratory, NY 2001).

Using the purified markers or their nucleic acid sequences, antibodies that specifically bind to a marker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

After the antibody is provided, a marker can be detected and/or quantified using any of suitable immunological binding assays known in the art (see, e.g., U.S. Pat. Nos. 4,366,241; 4,376,110; 4,517,288; and 4,837,168). Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Terr, eds., 7th ed. 1991); and Harlow & Lane, supra.

Generally, a sample obtained from a subject can be contacted with the antibody that specifically binds the marker. Optionally, the antibody can be fixed to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a probe substrate or microchip array. The sample is preferably a biological fluid sample taken from a subject. Examples of biological fluid samples include cerebrospinal fluid, blood, serum, plasma, neuronal cells, tissues, urine, tears, saliva etc. In a preferred embodiment, the biological fluid comprises cerebrospinal fluid. The sample can be diluted with a suitable eluant before contacting the sample to the antibody.

After incubating the sample with antibodies, the mixture is washed and the antibody-marker complex formed can be detected. This can be accomplished by incubating the washed mixture with a detection reagent. This detection reagent may be, e.g., a second antibody which is labeled with a detectable label. Exemplary detectable labels include magnetic beads (e.g., DYNABEADS™), fluorescent dyes, radiolabels, enzymes (e.g., horse radish peroxide, alkaline phosphatase and others commonly used in an ELISA), and colorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker is incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

Immunoassays can be used to determine presence or absence of a marker in a sample as well as the quantity of a marker in a sample. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can be determined by comparing to a standard. A standard can be, e.g., a known compound or another protein known to be present in a sample. As noted above, the test amount of marker need not be measured in absolute units, as long as the unit of measurement can be compared to a control.

The methods for detecting these markers in a sample have many applications. For example, one or more markers can be measured to aid in the diagnosis of spinal injury, brain injury, the degree of injury, tumor due to neuronal disorders, alcohol and drug abuse, fetal injury due to alcohol and/or drug abuse by pregnant mothers, etc. In another example, the methods for detection of the markers can be used to monitor responses in a subject to treatment. In another example, the methods for detecting markers can be used to assay for and to identify compounds that modulate expression of these markers in vivo or in vitro.

Data generated by desorption and detection of markers can be analyzed using any suitable means. In one embodiment, data is analyzed with the use of a programmable digital computer. The computer program generally contains a readable medium that stores codes. Certain code can be devoted to memory that includes the location of each feature on a probe, the identity of the adsorbent at that feature and the elution conditions used to wash the adsorbent. The computer also contains code that receives as input, data on the strength of the signal at various molecular masses received from a particular addressable location on the probe. This data can indicate the number of markers detected, including the strength of the signal generated by each marker.

Data analysis can include the steps of determining signal strength (e.g., height of peaks) of a marker detected and removing “outliers” (data deviating from a predetermined statistical distribution). The observed peaks can be normalized, a process whereby the height of each peak relative to some reference is calculated. For example, a reference can be background noise generated by instrument and chemicals (e.g., energy absorbing molecule) which is set as zero in the scale. Then the signal strength detected for each marker or other biomolecules can be displayed in the form of relative intensities in the scale desired (e.g., 100). Alternatively, a standard (e.g., a CSF protein) may be admitted with the sample so that a peak from the standard can be used as a reference to calculate relative intensities of the signals observed for each marker or other markers detected.

The computer can transform the resulting data into various formats for displaying. In one format, referred to as “spectrum view or retentate map,” a standard spectral view can be displayed, wherein the view depicts the quantity of marker reaching the detector at each particular molecular weight. In another format, referred to as “peak map,” only the peak height and mass information are retained from the spectrum view, yielding a cleaner image and enabling markers with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique markers and markers which are up- or down-regulated between samples. Marker profiles (spectra) from any two samples may be compared visually. In yet another format, Spotfire Scatter Plot can be used, wherein markers that are detected are plotted as a dot in a plot, wherein one axis of the plot represents the apparent molecular mass of the markers detected and another axis represents the signal intensity of markers detected. For each sample, markers that are detected and the amount of markers present in the sample can be saved in a computer readable medium. This data can then be compared to a control (e.g., a profile or quantity of markers detected in control, e.g., normal, healthy subjects in whom tumor is undetectable).

Diagnosis of Cancer

In another aspect, the invention provides methods for aiding a human tumor and/or tumor disorder diagnosis using one or more markers. For example, Eph receptors, Eph receptor related proteins, peptides, fragments and derivatives thereof, and ephrin molecules comprising any one of: ephrin A1, ephrin-A2, ephrin-A3, ephrin-A4, ephrin-A5, ephrin-B1, ephrin-B2 or ephrin-B3, variants and fragments thereof. These markers can be used singularly or in combination with other markers in any set, for example, CEA, Her2⁺. Many tumor antigens are well known in the art. See for example, Van den Eynde B J, van der Bruggen P. Curr Opin Immunol 1997; 9: 684-93; Houghton A N, Gold J S, Blachere N E. Curr Opin Immunol 2001; 13: 134-140; van der Bruggen P, Zhang Y, Chaux P, Stroobant V, Panichelli C, Schultz E S, Chapiro J, Van den Eynde B J, Brasseur F, Boon T. Immunol Rev 2002; 188: 51-64, which are herein incorporated by reference. Alternatively, many antibodies directed towards tumor antigens are commercially available.

Non-limiting examples of tumor antigens, include, tumor antigens resulting from mutations, such as: alpha-actinin-4 (lung carcinoma); BCR-ABL fusion protein (b3a2) (chronic myeloid leukemia); CASP-8 (head and neck squamous cell carcinoma); beta-catenin (melanoma); Cdc27 (melanoma); CDK4 (melanoma); dek-can fusion protein (myeloid leukemia); Elongation factor 2 (lung squamous carcinoa); ETV6-AML1 fusion protein (acute lymphoblastic leukemia); LDLR-fucosyltransferaseAS fusion protein (melanoma); overexpression of HLA-A2^{d} (renal cell carcinoma); hsp70-2 (renal cell carcinoma); KIAAO205 (bladder tumor); MART2 (melanoma); MUM-1f (melanoma); MUM-2 (melanoma); MUM-3 (melanoma); neo-PAP (melanoma); Myosin class I (melanoma); OS-9g (melanoma); pml-RARalpha fusion protein (promyelocytic leukemia); PTPRK (melanoma); K-ras (pancreatic adenocarcinoma); N-ras (melanoma). Examples of differentiation tumor antigens include, but not limited to: CEA (gut carcinoma); gp100/Pme117 (melanoma); Kallikrein 4 (prostate); mammaglobin-A (breast cancer); Melan-A/MART-1 (melanoma); PSA (prostate carcinoma); TRP-1/gp75 (melanoma); TRP-2 (melanoma); tyrosinase (melanoma). Over or under-expressed tumor antigens include but are not limited to: CPSF (ubiquitous); EphA3; G250/MN/CAIX (stomach, liver, pancreas); HER-2/neu; Intestinal carboxyl esterase (liver, intestine, kidney); alpha-foetoprotein (liver); M-CSF (liver, kidney); MUC1 (glandular epithelia); p53 (ubiquitous); PRAME (testis, ovary, endometrium, adrenals); PSMA (prostate, CNS, liver); RAGE-1 (retina); RU2AS (testis, kidney, bladder); survivin (ubiquitous); Telomerase (testis, thymus, bone marrow, lymph nodes); WT1 (testis, ovary, bone marrow, spleen); CA125 (ovarian).

The Eph biomarkers are differentially present in samples of a human patient, for example a cancer patient, such as one suffering from GBM, and a normal subject in whom tumor is undetectable. For example, some of the markers are expressed at an elevated level and/or are present at a higher frequency in human patients with tumor and/or cancer related disorders than in normal subjects. Therefore, detection of one or more of these markers in a person would provide useful information regarding the probability that the person may have tumor and/or cancer related disorder.

Other diseases which can be treated, prevented, and/or diagnosed with the compositions of the invention (e.g., polypeptides, polynucleotides, and/or agonists or antagonists), include, but are not limited to, nervous system injuries, and diseases, disorders, and/or conditions which result in either a disconnection of axons, a diminution or degeneration of neurons, or demyelination. Nervous system lesions which may be treated, prevented, and/or diagnosed in a patient (including human and non-human mammalian patients) according to the invention, include but are not limited to, the following lesions of either the central (including spinal cord, brain) or peripheral nervous systems: (1) ischemic lesions, in which a lack of oxygen in a portion of the nervous system results in neuronal injury or death, including cerebral infarction or ischemia, or spinal cord infarction or ischemia; (2) traumatic lesions, including lesions caused by physical injury or associated with surgery, for example, lesions which sever a portion of the nervous system, or compression injuries; (3) malignant lesions, in which a portion of the nervous system is destroyed or injured by malignant tissue which is either a nervous system associated malignancy or a malignancy derived from non-nervous system tissue; (4) infectious lesions, in which a portion of the nervous system is destroyed or injured as a result of infection, for example, by an abscess or associated with infection by human immunodeficiency virus, herpes zoster, or herpes simplex virus or with Lyme disease, tuberculosis, syphilis; (5) degenerative lesions, in which a portion of the nervous system is destroyed or injured as a result of a degenerative process including but not limited to degeneration associated with Parkinson's disease, Alzheimer's disease, Huntington's chorea, or amyotrophic lateral sclerosis (ALS); (6) lesions associated with nutritional diseases, disorders, and/or conditions, in which a portion of the nervous system is destroyed or injured by a nutritional disorder or disorder of metabolism including but not limited to, vitamin B12 deficiency, folic acid deficiency, Wernicke disease, tobacco-alcohol amblyopia, Marchiafava-Bignami disease (primary degeneration of the corpus callosum), and alcoholic cerebellar degeneration; (7) neurological lesions associated with systemic diseases including, but not limited to, diabetes (diabetic neuropathy, Bell's palsy), systemic lupus erythematosus, carcinoma, or sarcoidosis; (8) lesions caused by toxic substances including alcohol, lead, or particular neurotoxins; and (9) demyelinated lesions in which a portion of the nervous system is destroyed or injured by a demyelinating disease including, but not limited to, multiple sclerosis, human immunodeficiency virus-associated myelopathy, transverse myelopathy or various etiologies, progressive multifocal leukoencephalopathy, and central pontine myelinolysis.

Accordingly, embodiments of the invention include methods for diagnosing human tumor and/or cancer related disorders, wherein the method comprises: (a) detecting at least one marker in a sample, wherein the marker is selected from any one of Eph receptors, Eph-related receptors, peptides, fragments and derivatives thereof; and (b) correlating the detection of the marker or markers with a probable diagnosis of human tumor and/or cancer related disorder. The correlation may take into account the amount of the marker or markers in the sample compared to a control amount of the marker or markers (up or down regulation of the marker or markers) (e.g., in normal subjects in whom human tumor is undetectable). The correlation may take into account the presence or absence of the markers in a test sample and the frequency of detection of the same markers in a control. The correlation may take into account both of such factors to facilitate determination of whether a subject has tumor, the degree of severity of the tumor, and subcellular location of the injury, or not.

Any suitable samples can be obtained from a subject to detect markers. Preferably, a sample is a blood sample and/or cerebrospinal fluid sample from the subject. If desired, the sample can be prepared as described above to enhance detectability of the markers. For example, to increase the detectability of markers, a blood serum sample from the subject can be preferably fractionated by, e.g., Cibacron blue agarose chromatography and single stranded DNA affinity chromatography, anion exchange chromatography and the like. Sample preparations, such as pre-fractionation protocols, is optional and may not be necessary to enhance detectability of markers depending on the methods of detection used. For example, sample preparation may be unnecessary if antibodies that specifically bind markers are used to detect the presence of markers in a sample.

Any suitable method can be used to detect a marker or markers in a sample. For example, an immunoassay or gas phase ion spectrometry can be used as described above. Using these methods, one or more markers can be detected. Preferably, a sample is tested for the presence of a plurality of markers. Detecting the presence of a plurality of markers, rather than a single marker alone, would provide more information for the diagnostician. Specifically, the detection of a plurality of markers in a sample would increase the percentage of true positive and true negative diagnoses and would decrease the percentage of false positive or false negative diagnoses.

The detection of the marker or markers is then correlated with a probable diagnosis of tumor and/or cancer related disorders. In some embodiments, the detection of the mere presence or absence of a marker, without quantifying the amount of marker, is useful and can be correlated with a probable diagnosis of tumor and/or cancer related disorders. For example, tumor proteins, fragments or derivatives thereof, such as for example, Eph2A; can be more frequently detected in patients with cancer than in normal subjects.

In other embodiments, the detection of markers can involve quantifying the markers to correlate the detection of markers with a probable diagnosis of tumor, degree of severity of tumor, diagnosis of tumor disorders and the like. Thus, if the amount of the markers detected in a subject being tested is higher compared to a control amount, then the subject being tested has a higher probability of having a tumor.

Similarly, in another embodiment, the detection of markers can further involve quantifying the markers to correlate the detection of markers with a probable diagnosis of tumor, degree of severity of tumor, and the like, wherein the markers are present in lower quantities in CSF or blood serum samples from patients than in blood serum samples of normal subjects. Thus, if the amount of the markers detected in a subject being tested is lower compared to a control amount, then the subject being tested has a higher probability of having a tumor.

When the markers are quantified, it can be compared to a control. A control can be, e.g., the average or median amount of marker present in comparable samples of normal subjects in whom a tumor is undetectable. The control amount is measured under the same or substantially similar experimental conditions as in measuring the test amount. For example, if a test sample is obtained from a subject's cerebrospinal fluid and/or blood serum sample and a marker is detected using a particular probe, then a control amount of the marker is preferably determined from a serum sample of a patient using the same probe. It is preferred that the control amount of marker is determined based upon a significant number of samples from normal subjects who do not have tumor and/or neuronal disorders so that it reflects variations of the marker amounts in that population.

Data generated by mass spectrometry can then be analyzed by a computer software. The software can comprise code that converts signal from the mass spectrometer into computer readable form. The software also can include code that applies an algorithm to the analysis of the signal to determine whether the signal represents a “peak” in the signal corresponding to a marker of this invention, or other useful markers. The software also can include code that executes an algorithm that compares signal from a test sample to a typical signal characteristic of “normal” and human tumor and determines the closeness of fit between the two signals. The software also can include code indicating which the test sample is closest to, thereby providing a probable diagnosis.

Production of Antibodies to Detect Tumor Biomarkers

Tumor biomarkers obtained from samples in patients suffering from varying Eph⁺ tumors and the like, can be prepared as described above. Furthermore, tumor biomarkers can be subjected to enzymatic digestion to obtain fragments or peptides of the biomarkers for the production of antibodies to different antigenic epitopes that can be present in a peptide versus the whole protein. Antigenic epitopes are useful, for example, to raise antibodies, including monoclonal antibodies, that specifically bind the epitope. Antigenic epitopes can be used as the target molecules in immunoassays. (See, for instance, Wilson et al., Cell 37:767-778 (1984); Sutcliffe et al., Science 219:660-666 (1983)).

Tumor biomarker epitopes can be used, for example, to induce antibodies according to methods well known in the art. (See, for instance, Sutcliffe et al., supra; Wilson et al., supra; Chow et al., Proc. Natl. Acad. Sci. USA 82:910-914; and Bittle et al., J. Gen. Virol. 66:2347-2354 (1985). Tumor polypeptides comprising one or more immunogenic epitopes may be presented for eliciting an antibody response together with a carrier protein, such as an albumin, to an animal system (such as rabbit or mouse), or, if the polypeptide is of sufficient length (at least about 25 amino acids), the polypeptide may be presented without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting).

Epitope-bearing polypeptides of the present invention may be used to induce antibodies according to methods well known in the art including, but not limited to, in vivo immunization, in vitro immunization, and phage display methods. See, e.g., Sutcliffe et al., supra; Wilson et al., supra, and Bittle et al., J. Gen. Virol., 66:2347-2354 (1985). If in vivo immunization is used, animals may be immunized with free peptide; however, anti-peptide antibody titer may be boosted by coupling the peptide to a macromolecular carrier, such as keyhole limpet hemacyanin (KLH) or tetanus toxoid. For instance, peptides containing cysteine residues may be coupled to a carrier using a linker such as maleimidobenzoyl-N-hydroxysuccinimide ester (MBS), while other peptides may be coupled to carriers using a more general linking agent such as glutaraldehyde. Animals such as rabbits, rats and mice are immunized with either free or carrier-coupled peptides, for instance, by intraperitoneal and/or intradermal injection of emulsions containing about 100 μg of peptide or carrier protein and Freund's adjuvant or any other adjuvant known for stimulating an immune response. Several booster injections may be needed, for instance, at intervals of about two weeks, to provide a useful titer of anti-peptide antibody which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

Nucleic acid tumor biomarker epitopes can also be recombined with a gene of interest as an epitope tag (e.g., the hemaglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with the recombinant vaccinia virus are loaded onto Ni²⁺ nitriloacetic acid-agarose column and histidine-tagged proteins can be selectively eluted with imidazole-containing buffers.

The antibodies of the present invention may be generated by any suitable method known in the art. The antibodies of the present invention can comprise polyclonal antibodies. Methods of preparing polyclonal antibodies are known to the skilled artisan (Harlow, et al., Antibodies: A Laboratory Manual, (Cold spring Harbor Laboratory Press, 2nd ed. (1988), which is hereby incorporated herein by reference). For example, a polypeptide of the invention can be administered to various host animals including, but not limited to, rabbits, mice, rats, etc. to induce the production of sera containing polyclonal antibodies specific for the antigen. The administration of the polypeptides of the present invention may entail one or more injections of an immunizing agent and, if desired, an adjuvant. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art. For the purposes of the invention, “immunizing agent” may be defined as a polypeptide of the invention, including fragments, variants, and/or derivatives thereof, in addition to fusions with heterologous polypeptides and other forms of the polypeptides as may be described herein.

Typically, the immunizing agent and/or adjuvant will be injected in the mammal by multiple subcutaneous or intraperitoneal injections, though they may also be given intramuscularly, and/or through I.V. The immunizing agent may include polypeptides of the present invention or a fusion protein or variants thereof. Depending upon the nature of the polypeptides (i.e., percent hydrophobicity, percent hydrophilicity, stability, net charge, isoelectric point etc.), it may be useful to conjugate the immunizing agent to a protein known to be immunogenic in the mammal being immunized. Such conjugation includes either chemical conjugation by derivatizing active chemical functional groups to both the polypeptide of the present invention and the immunogenic protein such that a covalent bond is formed, or through fusion-protein based methodology, or other methods known to the skilled artisan. Examples of such immunogenic proteins include, but are not limited to keyhole limpet hemocyanin, serum albumin, bovine thyroglobulin, and soybean trypsin inhibitor. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Additional examples of adjuvants which may be employed includes the MPL-TDM adjuvant (monophosphoryl lipid A, synthetic trehalose dicorynomycolate). The immunization protocol may be selected by one skilled in the art without undue experimentation.

The antibodies of the present invention can also comprise monoclonal antibodies. Monoclonal antibodies may be prepared using hybridoma methods, such as those described by Kohler and Milstein, Nature, 256:495 (1975) and U.S. Pat. No. 4,376,110, by Harlow, et al., Antibodies: A Laboratory Manual, (Cold spring Harbor Laboratory Press, 2nd ed. (1988), by Hammerling, et al., Monoclonal Antibodies and T-Cell Hybridomas (Elsevier, N.Y., (1981)), or other methods known to the artisan. Other examples of methods which may be employed for producing monoclonal antibodies includes, but are not limited to, the human B-cell hybridoma technique (Kosbor et al., 1983, Immnunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In a hybridoma method, a mouse, a humanized mouse, a mouse with a human immune system, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include tumor polypeptides, fragments or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986), pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. As inferred throughout the specification, human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63). The culture medium in which the hybridoma cells are cultured can then be assayed for the presence of monoclonal antibodies directed against the tumor polypeptides of the present invention. Preferably, the binding specificity of monoclonal antibodies produced by the hybridoma cells is determined by immunoprecipitation or by an in vitro binding assay, such as radioimmunoassay (RIA) or enzyme-linked immunoadsorbant assay (ELISA). Such techniques are known in the art and within the skill of the artisan. The binding affinity of the monoclonal antibody can, for example, be determined by the Scatchard analysis of Munson and Pollart, Anal. Biochem., 107:220 (1980). After the desired hybridoma cells are identified, the clones may be subcloned by limiting dilution procedures and grown by standard methods (Goding, supra). Suitable culture media for this purpose include, for example, Dulbecco's Modified Eagle's Medium and RPMI-1640. Alternatively, the hybridoma cells may be grown in vivo as ascites in a mammal. The monoclonal antibodies secreted by the subclones may be isolated or purified from the culture medium or ascites fluid by conventional immunoglobulin purification procedures such as, for example, protein A-sepharose, hydroxyapatite chromatography, gel exclusion chromatography, gel electrophoresis, dialysis, or affinity chromatography.

The skilled artisan would acknowledge that a variety of methods exist in the art for the production of monoclonal antibodies and thus, the invention is not limited to their sole production in hybridomas. For example, the monoclonal antibodies may be made by recombinant DNA methods, such as those described in U.S. Pat. No. 4,816,567. In this context, the term “monoclonal antibody” refers to an antibody derived from a single eukaryotic, phage, or prokaryotic clone. The DNA encoding the monoclonal antibodies of the invention can be readily isolated and sequenced using conventional procedures (e.g., by using oligonucleotide probes that are capable of binding specifically to genes encoding the heavy and light chains of murine antibodies, or such chains from human, humanized, or other sources). The hybridoma cells of the invention serve as a preferred source of such DNA. Once isolated, the DNA may be placed into expression vectors, which are then transformed into host cells such as Simian COS cells, Chinese hamster ovary (CHO) cells, or myeloma cells that do not otherwise produce immunoglobulin protein, to obtain the synthesis of monoclonal antibodies in the recombinant host cells.

Methods for producing and screening for specific antibodies using hybridoma technology are routine and well known in the art. In a non-limiting example, mice can be immunized with a biomarker polypeptide or a cell expressing such peptide. Once an immune response is detected, e.g., antibodies specific for the antigen are detected in the mouse serum, the mouse spleen is harvested and splenocytes isolated. The splenocytes are then fused by well-known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention. The antibodies detecting tumor biomarkers, peptides and derivatives thereof, can be used in immunoassays and other methods to identify new tumor biomarkers and for use in the diagnosis of tumor, degree of severity of injury and/or neurological disorders.

Other methods can also be used for the large scale production of tumor biomarker specific antibodies. For example, antibodies can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference.

The antibodies of the present invention have various utilities. For example, such antibodies may be used in diagnostic assays to detect the presence or quantification of the polypeptides of the invention in a sample. Such a diagnostic assay can comprise at least two steps. The first, subjecting a sample with the antibody, wherein the sample is a tissue (e.g., human, animal, etc.), biological fluid (e.g., blood, urine, sputum, semen, amniotic fluid, saliva, etc.), biological extract (e.g., tissue or cellular homogenate, etc.), a protein microchip (e.g., See Arenkov P, et al., Anal Biochem., 278(2): 123-131 (2000)), or a chromatography column, etc. And a second step involving the quantification of antibody bound to the substrate. Alternatively, the method may additionally involve a first step of attaching the antibody, either covalently, electrostatically, or reversibly, to a solid support, and a second step of subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp 147-158). The antibodies used in the diagnostic assays can be labeled with a detectable moiety. The detectable moiety should be capable of producing, either directly or indirectly, a detectable signal. For example, the detectable moiety may be a radioisotope, such as ²H, ¹⁴C, ³²P, or ¹²⁵I, a florescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); David et al., Biochem., 13:1014 (1974); Pain et al., J. Immunol. Methods, 40:219(1981); and Nygren, J. Histochem. Cytochem., 30:407 (1982).

Identification of Nucleic Acid Sequences Expressing Tumor Biomarkers

A preferred embodiment of the invention is to identify genes and/or variants and correlate the effects of the protein encoded by these genes, when a patient is diagnosed with cancer. The identification of genes which can distinguish between susceptible and resistant individuals is important for distinguishing which nucleic acid sequences render individuals susceptible to Eph related cancers.

The genes identified from individuals are amplified by PCR and sequenced by methods well known in the art. These nucleic acid sequences are then used in the assays described in the examples and materials and methods to correlate the sequence of the genes identified with Eph+tumors. As more gene sequences and their amino acid sequences are identified, allows for a correlation between the effects of Eph2 expression and different gene sequences.

As more genes or variants thereof, are identified, oligonucleotide sequences are generated, or fragments thereof, may be employed as probes in the purification, isolation and detection of genes with similar sequences. Identification of a nucleic acid sequence capable of binding to a biomolecule of interest can be achieved by immobilizing a library of nucleic acids onto the substrate surface so that each unique nucleic acid was located at a defined position to form an array. The array would then be exposed to the biomolecule under conditions which favored binding of the biomolecule to the nucleic acids. Non-specifically binding biomolecules could be washed away using mild to stringent buffer conditions depending on the level of specificity of binding desired. The nucleic acid array would then be analyzed to determine which nucleic acid sequences bound to the biomolecule. Preferably the biomolecules would carry a fluorescent tag for use in detection of the location of the bound nucleic acids. Assays using an immobilized array of nucleic acid sequences may be used for determining the sequence of an unknown nucleic acid; single nucleotide polymorphism (SNP) analysis; analysis of gene expression patterns from a particular species, tissue, cell type, etc.; gene identification; etc. Any sequence can then be tested in macrophage viability assays described infra, or any other physical phenotypic criteria such as localization, MAP kinase 3 cleavage patterns and the like.

Other methods to determine the contributions of individual genes and or variants thereof, and their expression products. Genes or variants, thereof, can be isolated. Techniques are available to inactivate or alter any genetic region to any mutation desired by using targeted homologous recombination to insert specific changes into chromosomal variants. One approach for detecting homologous alteration events uses the polymerase chain reaction (PCR) to screen pools of transformant cells for homologous insertion, followed by screening individual clones (Kim et al., Nucleic Acids Res. 16:8887-8903 (1988); Kim et al, Gene 103:227-233 (1991)). Alternatively, a positive genetic selection approach has been developed in which a marker gene is constructed which will only be active if homologous insertion occurs, allowing these recombinants to be selected directly (Sedivy et al., Proc. Natl. Acad. Sci. USA 86:227-231 (1989)). One of the most general approaches developed for selecting homologous recombinants is the positive-negative selection (PNS) method developed for genes for which no direct selection of the alteration exists (Mansour et al., Nature 336:348-352: (1988); Capecchi, Science 244:1288-1292, (1989); Capecchi, Trends in Genet. 5:70-76 (1989)). The PNS method is more efficient for targeting genes that are not expressed at high levels because the marker gene has its own promoter. Nonhomologous recombinants are selected against by using the Herpes Simplex virus thymidine kinase (HSV-TK) gene and selecting against its nonhomologous insertion with the herpes drugs such as gancyclovir (GANC) or FIAU (1-(2-deoxy 2-fluoro-B-D-arabinofluranosyl)-5-iodouracil). By this counter-selection, the number of homologous recombinants in the surviving transformants can be enriched. Such transformants can be correlated with phenotypes as described infra.

Candidate Therapeutic Compounds and Compositions

In these methods, subjects are selected as described above, and tested for the identity of allelic variants of Eph or other genes identified according to the invention. The polynucleotides encoding Eph and allelic variants thereof, ephrins comprising any one of: ephrin A1, ephrin-A2, ephrin-A3, ephrin-A4, ephrin-A5, ephrin-B1, ephrin-B2 or ephrin-B3, variants and fragments thereof, may be used for diagnostic purposes. A variety of protocols for measuring Eph levels; including ELISAs, RIAs, and FACS, are known in the art and provide a basis for detecting Eph or its encoded product.

Other diagnostic methods include use of polynucleotides in a variety of methods. The polynucleotides which may be used include oligonucleotide sequences, complementary RNA and DNA molecules, and PNAs. The polynucleotides may be used to detect and quantitate gene expression in biopsied tissues in which expression of Eph may be correlated with susceptibility to cancer. The diagnostic assay may be used to determine absence, presence, and excess expression of Eph, and to monitor regulation of Eph levels during therapeutic intervention.

In a preferred embodiment, a method of identifying candidate therapeutic agents for treatment of tumors, comprises culturing an isolated cell expressing a receptor comprising any one of: EphA1, EphA2, EphA3, EphA4, EphA5, EphA6, EphA7, EphA8, EphB1, EphB2, EphB3, EphB4 and EphB5, eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk or nuk receptors, proteins, peptides, variants, fragments and derivatives thereof; and, administering a candidate therapeutic agent to the cultured cell; correlating expression levels and phosphorylation of the receptor in the presence or absence of a candidate therapeutic agent as compared to a normal cell and a cell cultured in the presence of an ephrin molecule; thereby, identifying candidate therapeutic agents that decrease Eph expression and activate Eph molecules, thereby, identifying candidate therapeutic agents for treatment of tumors. The activation of Eph molecules is determined by phosphorylation of tyrosine molecules as compared to Eph on a normal cell, Eph in the presence of its ephrin ligand and Eph in the presence of a candidate therapeutic agent. Phosphorylation assays for determining the activation or phosphorylation state are described in the Examples which follow. The expression of Eph is s compared to Eph in a normal cell, Eph+ cells in the presence of its ephrin ligand and Eph+ cells in the presence of a candidate therapeutic agent.

Another suitable method for diagnosis and candidate drug discovery includes contacting a test sample with the Eph gene, an allele or fragment thereof, or expression product of the Eph gene, an allele or fragment thereof; and detecting interaction of the test sample with the Eph gene, an allele or fragment thereof, or expression product of the Eph gene, an allele or fragment thereof. The test sample is a mammalian tissue or fluid (e.g. blood) sample. The Eph gene, an allele or fragment thereof, or expression product of the Eph gene, an allele or fragment thereof suitably can be detectably labeled e.g. with a fluorescent or radioactive component.

In another preferred embodiment, the ephrin molecule is a targeting ligand for candidate therapeutic compounds and is used to identify candidate therapeutic compounds. In a preferred embodiment, a method of identifying candidate therapeutic agents for treatment of tumors, comprises culturing an isolated cell expressing a receptor comprising any one of: ephrin A1, ephrin-A2, ephrin-A3, ephrin-A4, ephrin-A5, ephrin-B1, ephrin-B2 or ephrin-B3, variants and fragments thereof, administering a candidate therapeutic agent to the cultured cell; and, correlating expression levels and phosphorylation of the receptor in the presence or absence of a candidate therapeutic agent as compared to a normal cell and a cell cultured in the presence of an ephrin molecule; thereby, identifying candidate therapeutic agents that bind to ephrin molecules, and identifying candidate therapeutic agents for treatment of tumors. Preferably, the candidate therapeutic agents are administered to normal cells and cancer cells and assayed for binding to ephrin molecules. Appropriate controls are used, such as for example, binding of the candidate therapeutic agents to ephrin molecules is compared to binding of said agents to normal cells and cancer cells. The effects of these candidate therapeutic agents have by binding to ephrin molecules is compared to expression and activation of Eph molecules, wherein activation of Eph molecules is determined by phosphorylation of tyrosine molecules as compared to Eph on a normal cell, Eph in the presence of its ephrin ligand and Eph in the presence of a candidate therapeutic agent. Preferably, expression of Eph is compared to Eph in a normal cell, Eph+ cells in the presence of its ephrin ligand and Eph+ cells in the presence of a candidate therapeutic agent. An example of identifying candidate therapeutic molecules which bind to ephrin is described in the Examples which follow.

In one aspect, hybridization with oligonucleotide probes that are capable of detecting polynucleotide sequences, including genomic sequences, encoding Eph or closely related molecules may be used to identify nucleic acid sequences which encode Eph. The specificity of the probe, whether it is made from a highly specific region, e.g., the 5′ regulatory region, or from a less specific region, e.g., a conserved motif, and the stringency of the hybridization or amplification (maximal, high, intermediate, or low), will determine whether the probe identifies only naturally occurring sequences encoding Eph, allelic variants, or related sequences.

Probes may also be used for the detection of related sequences, and should preferably have at least 50% sequence identity or homology to any of the Eph encoding sequences, more preferably at least about 60, 70, 75, 80, 85, 90 or 95 percent sequence identity to any of the Eph encoding sequences (sequence identity determinations discussed above, including use of BLAST program). The hybridization probes of the subject invention may be DNA or RNA and may be derived from the sequences of the invention or from genomic sequences including promoters, enhancers, and introns of the Eph gene. “Homologous”, as used herein, refers to the subunit sequence similarity between two polymeric molecules, e.g., between two nucleic acid molecules such as two DNA molecules, or two polypeptide molecules. When a subunit position in both of the two molecules is occupied by the same monomeric subunit (e.g., if a position in each of two DNA molecules is occupied by adenine) then they are homologous at that position. The homology between two sequences is a direct function of the number of matching or homologous positions. For example, if 5 of 10 positions in two compound sequences are matched or homologous then the two sequences are 50% homologous, if 9 of 10 are matched or homologous, the two sequences share 90% homology. By way of example, the DNA sequences 3′ ATTGCC 5′ and 3′ TTTCCG 5′ share 50% homology.

Means for producing specific hybridization probes for DNAs encoding Eph include the cloning of polynucleotide sequences encoding Eph or Eph derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³²S, or by enzymatic labels, such as alkaline phosphatase coupled to the probe via avidin-biotin coupling systems, fluorescent labeling, and the like.

The polynucleotide sequences encoding Eph may be used in Southern or Northern analysis, dot blot, or other membrane-based technologies; in PCR technologies; in dipstick, pin, and multiformat ELISA-like assays; and in microarrays utilizing fluids or tissues from patients to detect altered Eph expression. Gel-based mobility-shift analyses may be employed. Other suitable qualitative or quantitative methods are well known in the art.

Identity of genes, or variants thereof, can be verified using techniques well known in the art. Examples include but are not limited to, nucleic acid sequencing of amplified genes, hybridization techniques such as single nucleic acid polymorphism analysis (SNP), microarrays wherein the molecule of interest is immobilized on a biochip. Overlapping cDNA clones can be sequenced by the dideoxy chain reaction using fluorescent dye terminators and an ABI sequencer (Applied Biosystems, Foster City, Calif.). Any type of assay wherein one component is immobilized may be carried out using the substrate platforms of the invention. Bioassays utilizing an immobilized component are well known in the art. Examples of assays utilizing an immobilized component include for example, immunoassays, analysis of protein-protein interactions, analysis of protein-nucleic acid interactions, analysis of nucleic acid-nucleic acid interactions, receptor binding assays, enzyme assays, phosphorylation assays, diagnostic assays for determination of disease state, genetic profiling for drug compatibility analysis, SNP detection, etc.

An Eph receptor or Eph receptor related gene means the gene and all currently known variants thereof, including the different mRNA transcripts to which the gene and its variants can give rise, and any further gene variants which may be elucidated. Exemplary EPH receptors include the EphA1, EphA2, EphA3, EphA4, EphA5, EphA6, EphA7, EphA8, EphB1, EphB2, EphB3, EphB4 and EphB5, eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk and nuk receptors. Ligands include, but not limited to, ephrin-A1, ephrin-A2, ephrin-A3, ephrin-A4, ephrin-A5, ephrin-B1, ephrin-B2 and ephrin-B3.

In general, however, such variants will have significant homology (sequence identity) to a sequence of Eph, e.g. a variant will have at least about 70 percent homology (sequence identity) to a sequence of Eph, more typically at least about 75, 80, 85, 90, 95, 97, 98 or 99 homology (sequence identity) to a sequence of Eph. Homology of a variant can be determined by any of a number of standard techniques such as a BLAST program.

Identification of a nucleic acid sequence capable of binding to a biomolecule of interest can be achieved by immobilizing a library of nucleic acids onto the substrate surface so that each unique nucleic acid was located at a defined position to form an array. The array would then be exposed to the biomolecule under conditions which favored binding of the biomolecule to the nucleic acids. Non-specifically binding biomolecules could be washed away using mild to stringent buffer conditions depending on the level of specificity of binding desired. The nucleic acid array would then be analyzed to determine which nucleic acid sequences bound to the biomolecule. Preferably the biomolecules would carry a fluorescent tag for use in detection of the location of the bound nucleic acids.

An assay using an immobilized array of nucleic acid sequences may be used for determining the sequence of an unknown nucleic acid; single nucleotide polymorphism (SNP) analysis; analysis of gene expression patterns from a particular species, tissue, cell type, etc.; gene identification; etc.

Additional diagnostic uses for oligonucleotides designed from the sequences encoding Eph may involve the use of PCR. These oligomers may be chemically synthesized, generated enzymatically, or produced in vitro. Oligomers will preferably contain a fragment of a polynucleotide encoding Eph, or a fragment of a polynucleotide complementary to the polynucleotide encoding Eph, and will be employed under optimized conditions for identification of a specific gene. Oligomers may also be employed under less stringent conditions for detection or quantitation of closely-related DNA or RNA sequences.

High stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH and nucleic acid concentration) at which 50% of a nucleic acid sequence complementary to the target, hybridizes to the target sequence at equilibrium. Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short nucleic acid sequences (e.g. 10 to 50 nucleotides) and at least about 60° C. for long nucleic acid sequences (e.g. greater than 50 nucleotides). Stringent conditions may also be achieved with the addition of destabilizing agents such as formamide.

The phrase “stringent hybridization” is used herein to refer to conditions under which polynucleic acid hybrids are stable. As known to those of skill in the art, the stability of hybrids is reflected in the melting temperature (Tₘ) of the hybrids. In general, the stability of a hybrid is a function of sodium ion concentration and temperature. Typically, the hybridization reaction is performed under conditions of lower stringency, followed by washes of varying, but higher, stringency. Reference to hybridization stringency relates to such washing conditions. As used herein, the phrase “moderately stringent hybridization” refers to conditions that permit target-DNA to bind a complementary nucleic acid that has about 60% identity, preferably about 75% identity, more preferably about 85% identity to the target DNA; with greater than about 90% identity to target-DNA being especially preferred. Preferably, moderately stringent conditions are conditions equivalent to hybridization in 50% formamide, 5× Denhart's solution, 5×SSPE, 0.2% SDS at 42° C., followed by washing in 0.2×SSPE, 0.2% SDS, at 65° C.

The phrase “high stringency hybridization” refers to conditions that permit hybridization of only those nucleic acid sequences that form stable hybrids in, for example, 0.018 M NaCl at 65° C. (i.e., if a hybrid is not stable in 0.018 M NaCl at 65° C., it will not be stable under high stringency conditions). High stringency conditions can be provided, for example, by hybridization in 50% formamide, 5× Denhart's solution, 5×SSPE, 0.2% SDS at 42° C., followed by washing in 0.1×SSPE, and 0.1% SDS at 65° C.

The phrase “low stringency hybridization” refers to conditions equivalent to hybridization in 10% formamide, 5× Denhart's solution, 6×SSPE, 0.2% SDS at 42° C., followed by washing in 1×SSPE, 0.2% SDS, at 50° C. Denhart's solution and SSPE (see, e.g., Sambrook et al., Molecular Cloning, A Laboratory Manual, Cold Spring Harbor Laboratory Press, 1989) are well known to those of skill in the art as are other suitable hybridization buffers.

In further embodiments, oligonucleotides or longer fragments derived from any of the Eph mouse or human polynucleotide sequences described herein, may be used as targets in a microarray. The microarray can be used to monitor the identity and/or expression level of large numbers of genes and gene transcripts simultaneously to identify genes with which Eph or its product interacts and/or to assess the efficacy of candidate therapeutic agents in regulating genes that mediate EphA2⁺ receptor tumor susceptibility. Microarrays may be used to particular advantage in diagnostic assays, to identify genetic variants, mutations, and polymorphisms of genes that mediate EphA2 tumor susceptibility in a biological sample from a mammal, such as a human or other research subject or clinical patient. This information may be used to determine gene function, and to develop and monitor the activities of therapeutic agents.

In other embodiments, oligonucleotides or longer fragments derived from any of the polynucleotide sequences herein (including, in non-limiting fashion, human sequences) and genomic sequences adjacent to them may be used as diagnostic reagents, such as to detect single-nucelotide polymorphisms or other variations or mutations in Eph or a homologous gene, amplification of Eph or homologous nucleic acid sequences, and for use in nucleic acid sequencing methods.

Microarrays may be prepared, used, and analyzed using methods known in the art (see, e.g., Brennan et al., 1995, U.S. Pat. No. 5,474,796; Schena et al., 1996, Proc. Natl. Acad. Sci. U.S.A. 93: 10614-10619; Baldeschweiler et al., 1995, PCT application WO95/251116; Shalon, et al., 1995, PCT application WO95/35505; Heller et al., 1997, Proc. Natl. Acad. Sci. U.S.A. 94: 2150-2155; and Heller et al., 1997, U.S. Pat. No. 5,605,662).

Expression or activity levels for Eph also may be examined. Normal or standard values for Eph expression are established by combining body fluids or cell extracts taken from normal mammalian subjects, preferably human, with antibody to Eph under conditions suitable for complex formation. The amount of standard complex formation may be quantitated by various methods, preferably by photometric means. Quantities of Eph expressed in subject, control, and disease samples from biopsied tissues are compared with the standard values. Deviation between standard and subject values establishes the parameters for Eph biomarker expression. Parameters studied include, but are not limited to, the below and those described throughout the specification.

Candidate agents include numerous chemical classes, though typically they are organic compounds including small organic compounds, nucleic acids including oligonucleotides, and peptides. Small organic compounds suitably may have e.g. a molecular weight of more than about 40 or 50 yet less than about 2,500. Candidate agents may comprise functional chemical groups that interact with proteins and/or DNA.

Candidate agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of e.g. bacterial, fungal and animal extracts are available or readily produced.

Therapeutic agent assays of the invention suitably include, animal models, cell-based systems and non-cell based systems.

Preferably, Eph, variants, fragments, or oligopeptides thereof are used for identifying agents of therapeutic interest, e.g. by screening libraries of compounds or otherwise identifying compounds of interest by any of a variety of drug screening or analysis techniques. The Eph, allele, fragment, or oligopeptide thereof employed in such screening may be free in solution, affixed to a solid support, borne on a cell surface, or located intracellularly. The formation of binding complexes between Eph and the agent being tested may be measured and then tested.

Another technique for drug screening provides for high throughput screening of compounds having suitable binding affinity to the protein of interest (see, e.g., Geysen et al., 1984, PCT application WO84/03564). In this method, large numbers of different small test compounds are synthesized on a solid substrate. The test compounds are reacted with Eph, or fragments thereof, and washed. Bound Eph is then detected by methods well known in the art. Purified Eph can also be coated directly onto plates for use in the aforementioned drug screening techniques. Alternatively, non-neutralizing antibodies can be used to capture the peptide and immobilize it on a solid support.

Expression Vectors

As discussed above, a preferred use of nucleic acid sequences identified in the present invention, is for the generation of treatments that ameliorate tumors. The Eph receptor and Eph-receptor related genes can be expressed by a vector containing a DNA segment encoding the wild-type, alleles, variants, mutations or fragments of the genes. Mutations and alleles of the Eph genes are also preferably used in the construction of a vector for use in treatment. The vector comprising the desired nucleic acid sequence expressing Eph²⁺, preferably has at least one such nucleic acid sequence. Alternatively, the vector may be comprised of more than one such nucleic acid sequence, or combinations of allelic variants. The vector can also be comprised of cassettes of different allelic variants or wild type Eph genes.

The fourteen members of the Eph receptors are divided into A and B classes based on the similarity of their extracellular domains and their ability to interact with their membrane-bound ligands, the ephrins. Endogenous ligands of Eph receptors are anchored to the surface of neighboring cells makes them unique among the receptor tyrosine kinases, which typically bind soluble factors such as epidermal growth factor (EGF) and vascular endothelial growth factor (VEGF).

As discussed above, most Eph receptors play an important role in axon guiding during cancer development through the mediation of contact-dependent processes between cells. EphA2, however, is normally expressed at low levels on the surface of adult epithelial cells. It is localized to intercellular junctions, where virtually all of the receptor is bound by its ligand, ephrinA1. EphA2 is significantly overexpressed in several human epithelial cancers, including colon, breast, , and pancreatic carcinoma. The unstable cell-cell contacts in cancer tissue hinder the ability of EphA2 to interact with ephrinA1 on neighboring cells. As a result, receptor activation as well as ephrinA1-induced EphA2 degradation are markedly decreased. Interestingly, this produces a situation in malignant cells in which EphA2 is significantly less activated, and at the same time, highly overexpressed. Many of the invasive and aggressive phenotypes of cancer are directly correlated with the overexpression of EphA2, both in vitro and in vivo.

According to the present invention, the coding sequence on the plasmid that encodes the Eph genes or ephrin A1 is provided with a coding sequence that encodes an amino acid sequence whose presence on the protein results in a specific intracellular localization of the expressed protein.

Introducing the genes, fragments or variants thereof, into an individual can include use of vectors, liposomes, naked DNA, adjuvant-assisted DNA, gene gun, catheters, etc. Vectors include chemical conjugates such as described in WO 93/04701, which has a targeting moiety (e.g. a ligand to a cellular surface receptor), and a nucleic acid binding moiety (e.g. polylysine), viral vector (e.g. a DNA or RNA viral vector), fusion proteins such as described in PCT/US95/02140 (WO 95/22618) which is a fusion protein containing a target moiety (e.g. an antibody specific for a target cell) and a nucleic acid binding moiety (e.g. a protamine), plasmids, phage etc. The vectors can be chromosomal, non-chromosomal or synthetic.

Preferred nucleic acid sequences that encode for Eph and/or ephrin A1 may suitably comprise any of the Eph receptors and their ligands, as well as sequences that have a substantial sequence identity to Eph, e.g. at least about 70, 75, 80, 85, 90 or 95 percent sequence identity to any one or more of those sequences. Also preferred nucleic acid sequences that encode for a modified Epha2 amino acid sequence comprise a sequence that will hybridize under normal or high stringency conditions (as such conditions are defined immediately below) to any of the Eph genes and their ligands such as for example, ephrin A1.

Preferred vectors include viral vectors, fusion proteins and chemical conjugates. Retroviral vectors include Moloney murine leukemia viruses. DNA viral vectors are preferred. Viral vectors can be chosen to introduce the genes to cells of choice. Such vectors include pox vectors such as orthopox or avipox vectors, herpesvirus vectors such as herpes simplex I virus (HSV) vector (Geller et al., 1995, J. Neurochem. 64: 487; Lim et al., 1995, in DNA Cloning: Mammalian Systems, D. Glover, ed., Oxford Univ. Press, Oxford, England; Geller et al., 1990, Proc. Natl. Acad. Sci. U.S.A. 87: 1149), adenovirus vectors (LeGal LaSalle et al., 1993, Science 259: 988; Davidson et al., 1993, Nat. Genet. 3: 219; Yang et al., 1995, J. Virol. 69: 2004) and adeno-associated virus vectors (Kaplitt et al., 1994, Nat. Genet. 8: 148).

Pox viral vectors introduce the gene into the cells cytoplasm. Avipox virus vectors result in only short term expression of the nucleic acid. Adenovirus vectors, adeno-associated virus vectors and herpes simplex virus vectors are preferred for introducing the nucleic acid into neural cells. The adenovirus vector results in a shorter term expression (about 2 months) than adeno-associated virus (about 4 months), which in turn is shorter than HSV vectors. The vectors can be introduced by standard techniques, e.g. infection, transfection, transduction or transformation. Examples of modes of gene transfer include for example, naked DNA calcium phosphate precipitation, DEAE dextran, electroporation, protoplast fusion, lipofection, cell microinjection and viral vectors.

The vector can be employed to target essentially any desired target cell. For example, stereotaxic injection can be used to direct the vectors (e.g. adenovirus, HSV) to a desired location. Other methods that can be used include catheters, intravenous, parenteral, intraperitoneal, and subcutaneous injection, and oral or other known routes of administration.

Another preferred method is DNA immunization. DNA immunization employs the subcutaneous injection of a plasmid DNA (pDNA) vector encoding a specific Eph protein and/or ligands, such as for example, ephrin A1. The pDNA sequence is taken up by antigen presenting cells (APC). Once inside the cell, the DNA encoding protein is transcribed and translated. Genetic constructs comprise a nucleotide sequence that encodes the Eph, ephrin A1 nucleic acid sequence of choice and preferably includes an intracellular trafficking sequence operably linked to regulatory elements needed for gene expression.

When taken up by a cell, the genetic construct(s) may remain present in the cell as a functioning extrachromosomal molecule and/or integrate into the cell's chromosomal DNA. DNA may be introduced into cells where it remains as separate genetic material in the form of a plasmid or plasmids. Alternatively, linear DNA which can integrate into the chromosome may be introduced into the cell. When introducing DNA into the cell, reagents which promote DNA integration into chromosomes may be added. DNA sequences which are useful to promote integration may also be included in the DNA molecule. Alternatively, RNA may be administered to the cell. It is also contemplated to provide the genetic construct as a linear minichromosome including a centromere, telomeres and an origin of replication. Gene constructs may remain part of the genetic material in attenuated live microorganisms or recombinant microbial vectors which live in cells. Gene constructs may be part of genomes of recombinant viral vaccines where the genetic material either integrates into the chromosome of the cell or remains extrachromosomal.

Genetic constructs include regulatory elements necessary for gene expression of a nucleic acid molecule. The elements include: a promoter, an initiation codon, a stop codon, and a polyadenylation signal. In addition, enhancers may be required for gene expression of the sequence of choice, for example, the Eph gene, variants or fragments thereof. It is necessary that these elements be operably linked to the sequence that encodes the desired proteins and that the regulatory elements are operable in the individual to whom they are administered.

Initiation codons and stop codons are generally considered to be part of a nucleotide sequence that encodes the immunogenic target protein. However, it is necessary that these elements are functional in the individual to whom the gene construct is administered. The initiation and termination codons must be in frame with the coding sequence.

Promoters and polyadenylation signals used must be functional within the cells of the individual.

Examples of promoters useful to practice the present invention, especially in the production of a genetic vaccine for humans, include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from human genes such as human Actin, human Myosin, human Hemoglobin, human muscle creatine and human metallothionein.

Examples of polyadenylation signals useful to practice the present invention, especially in the production of a genetic vaccine for humans, include but are not limited to SV40 polyadenylation signals and LTR polyadenylation signals. In particular, the SV40 polyadenylation signal which is in pCEP4 plasmid (Invitrogen, San Diego Calif.), referred to as the SV40 polyadenylation signal, is used.

In addition to the regulatory elements required for DNA expression, other elements may also be included in the DNA molecule. Such additional elements include enhancers. The enhancer may be selected from the group including but not limited to: human Actin, human Myosin, human Hemoglobin, human muscle creatine and viral enhancers such as those from CMV, RSV and EBV.

Genetic constructs can be provided with mammalian origin of replication in order to maintain the construct extrachromosomally and produce multiple copies of the construct in the cell. For example, plasmids pCEP4 and pREP4 from Invitrogen (San Diego, Calif.) contain the Epstein Barr virus origin of replication and nuclear antigen EBNA-1 coding region which produces high copy episomal replication without integration.

In order to maximize protein production, regulatory sequences may be selected which are well suited for gene expression in the cells the construct is administered into. Moreover, codons may be selected which are most efficiently transcribed in the cell. One having ordinary skill in the art can produce DNA constructs which are functional in the cells.

The method of the present invention comprises the steps of administering nucleic acid molecules to tissue of the individual. In some preferred embodiments, the nucleic acid molecules are administered intramuscularly, intranasally, intraperatoneally, subcutaneously, intradermally, or topically or by lavage to mucosal tissue selected from the group consisting of vaginal, rectal, urethral, buccal and sublingual.

In some embodiments, the nucleic acid molecule is delivered to the cells in conjunction with administration of a facilitating agent. Facilitating agents are also referred to as polynucleotide function enhancers or genetic vaccine facilitator agents. Facilitating agents are described in e.g. International Application No. PCT/US94/00899 filed Jan. 26, 1994 and International Application No. PCT/US95/04071 filed Mar. 30, 1995, both incorporated herein by reference. Facilitating agents which are administered in conjunction with nucleic acid molecules may be administered as a mixture with the nucleic acid molecule or administered separately simultaneously, before or after administration of nucleic acid molecules.

In some preferred embodiments, the genetic constructs of the invention are formulated with or administered in conjunction with a facilitator selected from the group consisting of, for example, benzoic acid esters, anilides, amidines, urethans and the hydrochloride salts thereof such as those of the family of local anesthetics. The facilitating agent is administered prior to, simultaneously with or subsequent to the genetic construct. The facilitating agent and the genetic construct may be formulated in the same composition.

In some embodiments of the invention, the individual is first subject to injection of the facilitator prior to administration of the genetic construct. That is, for example, up to a about a week to ten days prior to administration of the genetic construct, the individual is first injected with the facilitator. In some embodiments, the individual is injected with the facilitator about 1 to 5 days; in some embodiments 24 hours, before or after administration of the genetic construct. Alternatively, if used at all, the facilitator is administered simultaneously, minutes before or after administration of the genetic construct. Accordingly, the facilitator and the genetic construct may be combined to form a single pharmaceutical composition.

In some embodiments, the genetic constructs are administered free of facilitating agents, that is in formulations free from facilitating agents using administration protocols in which the genetic constructions are not administered in conjunction with the administration of facilitating agents.

Nucleic acid molecules which are delivered to cells according to the invention may serve as genetic templates for proteins that function as prophylactic and/or therapeutic immunizing agents. In preferred embodiments, the nucleic acid molecules comprise the necessary regulatory sequences for transcription and translation of the coding region in the cells of the animal.

To further define nucleic acid sequences important for conferring resistance to Eph related tumors, the invention provides for mutants of Eph and/or ephrin A1. Additionally, the Eph protein-encoding nucleic acid sequences of choice can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, in vitro site-directed mutagenesis (Hutchinson et al., 1978, J. Biol. Chem. 253: 6551; Zoller and Smith, 1984, DNA 3:479-488; Oliphant et al., 1986, Gene 44: 177; Hutchinson et al., 1986, Proc. Natl. Acad. Sci. U.S.A. 83: 710; and others). PCR techniques are preferred for site directed mutagenesis (see Higuchi, 1989, “Using PCR to Engineer DNA”, in PCR Technology: Principles and Applications for DNA Amplification, H. Erlich, ed., Stockton Press, Chapter 6, pp. 61-70).

Various methods known to those skilled in the art can be used to express and produce nucleic acid sequences conferring resistance to EphA2⁺ tumors. For example, the identified and isolated gene can be inserted into an appropriate cloning vector. A large number of vector-host systems known in the art may be used. Possible vectors include, but are not limited to, plasmids or modified viruses, but the vector system must be compatible with the host cell used. Examples of vectors include, but are not limited to, E. coli, bacteriophages such as lambda derivatives, or plasmids such as pBR322 derivatives or pUC plasmid derivatives, e.g., pGEX vectors, pmal-c, pFLAG, etc. The insertion into a cloning vector can, for example, be accomplished by ligating the DNA fragment into a cloning vector that has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences from the yeast 2μ plasmid.

In an alternative method, the desired gene may be identified and isolated after insertion into a suitable cloning vector in a “shot gun” approach. Enrichment for the desired gene, for example, by size fractionation, removal of highly-repetitive sequences, subtractive or otherwise selective hybridization, and other methods as may be known in the art, can be done before insertion into the cloning vector.

The nucleotide sequence coding for Eph protein, ephrin A1, functional fragments, derivatives or analogs thereof, including a chimeric protein, thereof, can be inserted into an appropriate expression vector, i.e., a vector which contains the necessary elements for the transcription and translation of the inserted protein-coding sequence. Such elements are termed herein a “promoter.” Thus, the nucleic acid encoding a Eph protein of the invention or functional fragment, derivatives or analogs thereof, is operationally associated with a promoter in an expression vector of the invention. Both cDNA and genomic sequences can be cloned and expressed under control of such regulatory sequences. An expression vector also preferably includes a replication origin. The necessary transcriptional and translational signals can be provided on a recombinant expression vector.

Potential host-vector systems include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

A recombinant Eph protein of the invention, may be expressed chromosomally, after integration of the coding sequence by recombination. In this regard, any of a number of amplification systems may be used to achieve high levels of stable gene expression (See Sambrook et al., 1989, supra).

The cell into which the recombinant vector comprising the nucleic acid encoding Eph protein, ephrin A1 is cultured in an appropriate cell culture medium under conditions that provide for expression of Eph protein by the cell.

Any of the methods previously described for the insertion of DNA fragments into a cloning vector may be used to construct expression vectors containing a gene consisting of appropriate transcriptional/translational control signals and the protein coding sequences. These methods may include in vitro recombinant DNA and synthetic techniques and in vivo recombination (genetic recombination).

Expression of Eph protein, ephrin A1 may be controlled by any promoter/enhancer element known in the art, but these regulatory elements must be functional in the host selected for expression.

Expression vectors containing a nucleic acid encoding an Eph protein, ephrin A1 of the invention can be detected or identified by four general approaches: (a) PCR amplification of the desired plasmid DNA or specific mRNA, (b) nucleic acid hybridization, (c) presence or absence of selection marker gene functions, and (d) expression of inserted sequences. In the first approach, the nucleic acids can be amplified by PCR to provide for detection of the amplified product. In the second approach, the presence of a foreign gene inserted in an expression vector can be detected by nucleic acid hybridization using probes comprising sequences that are homologous to an inserted marker gene. In the third approach, the recombinant vector/host system can be identified and selected based upon the presence or absence of certain “selection marker” gene functions (e.g., β-galactosidase activity, thymidine kinase activity, resistance to antibiotics, transformation phenotype, occlusion body formation in baculovirus, etc.) caused by the insertion of foreign genes in the vector. In another example, if the nucleic acid encoding Eph protein, ephrin A1 is inserted within the “selection marker” gene sequence of the vector, recombinants containing the Eph protein insert can be identified by the absence of the Eph protein gene function.

A wide variety of host/expression vector combinations may be employed in expressing the DNA sequences of this invention. Useful expression vectors, for example, may consist of segments of chromosomal, nonchromosomal and synthetic DNA sequences. Suitable vectors include derivatives of SV40 and known bacterial plasmids, e.g., E. coli plasmids col E1, pCR1, pBR322, pMal-C2, pET, pGEX (Smith et al., 1988, Gene 67: 31-40), pMB9 and their derivatives, plasmids such as RP4; phage DNAS, e.g., the numerous derivatives of λ phage, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

For example, in a baculovirus expression systems, both non-fusion transfer vectors, such as but not limited to pVL941 (BamH1 cloning site; Summers), pVL1393 (BamH1, SmaI, XbaI, EcoR1, NotI, XmaIII, BglII, and PstI cloning site; Invitrogen), pVL1392 (BglII, PstI, NotI, XmaIII, EcoRI, XbaI, SmaI, and BamH1 cloning site; Summers and Invitrogen), and pBlueBacIII (BamH1, BglII, PstI, NcoI, and HindIII cloning site, with blue/white recombinant screening possible; Invitrogen), and fusion transfer vectors, such as but not limited to pAc700 (BamH1 and KpnI cloning site, in which the BamH1 recognition site begins with the initiation codon; Summers), pAc701 and pAc702 (same as pAc700, with different reading frames), pAc360 (BamH1 cloning site 36 base pairs downstream of a polyhedron initiation codon; Invitrogen(195)), and pBlueBacHisA, B, C (three different reading frames, with BamH1, BglII, PstI, NcoI, and HindIII cloning site, an N-terminal peptide for ProBond purification, and blue/white recombinant screening of plaques; Invitrogen (220)) can be used.

Mammalian expression vectors contemplated for use in the invention include vectors with inducible promoters, such as the dihydrofolate reductase (DHFR) promoter, e.g., any expression vector with a DHFR expression vector, or a DHFR/methotrexate co-amplification vector, such as pED (PstI, SalI, SbaI, SmaI, and EcoRI cloning site, with the vector expressing both the cloned gene and DHFR; see Kaufman, Current Protocols in Molecular Biology, 16.12 (1991). Alternatively, a glutamine synthetase/methionine sulfoximine co-amplification vector, such as pEE14 (HindIII, XbaI, SmaI, SbaI, EcoRI, and BclI cloning site, in which the vector expresses glutamine synthase and the cloned gene; Celltech). In another embodiment, a vector that directs episomal expression under control of Epstein Barr Virus (EBV) can be used, such as pREP4 (BamH1, SfiI, XhoI, NotI, NheI, HindIII, NheI, PvuII, and KpnI cloning site, constitutive RSV-LTR promoter, hygromycin selectable marker; Invitrogen), pCEP4 (BamH1, SfiI, XhoI, NotI, NheI, HindIII, NheI, PvuII, and KpnI cloning site, constitutive hCMV immediate early gene, hygromycin selectable marker; Invitrogen), pMEP4 (KpnI, PvuI, NheI, HindIII, NotI, XhoI, SfiI, BamH1 cloning site, inducible methallothionein IIa gene promoter, hygromycin selectable marker: Invitrogen), pREP8 (BamH1, XhoI, NotI, HindIII, NheI, and KpnI cloning site, RSV-LTR promoter, histidinol selectable marker; Invitrogen), pREP9 (KpnI, NbeI, HindIII, NotI, XhoI, SfiI, and BamHI cloning site, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen). Selectable mammalian expression vectors for use in the invention include pRc/CMV (HindIII, BstXI, NotI, SbaI, and ApaI cloning site, G418 selection; Invitrogen), pRc/RSV (HindIII, SpeI, BstXI, NotI, XbaI cloning site, G418 selection; Invitrogen), and others. Vaccinia virus mammalian expression vectors (see, Kaufman, 1991, supra) for use according to the invention include but are not limited to pSC11 (SmaI cloning site, TK- and β-gal selection), pMJ601 (SalI, SmaI, AflI, NarI, BspMII, BamHI, ApaI, NheI, SacII, KpnI, and HindIII cloning site; TK- and β-gal selection), and pTKgptF1S (EcoRI, PstI, SalI, AccI, HindII, SbaI, BamHI, and Hpa cloning site, TK or XPRT selection).

Yeast expression systems can also be used according to the invention to express Eph polypeptides. For example, the non-fusion pYES2 vector (XbaI, SphI, ShoI, NotI, GstXI, EcoRI, BstXI, BamH1, SacI, Kpn1, and HindIII cloning sit; Invitrogen) or the fusion pYESHisA, B, C (XbaI, SphI, ShoI, NotI, BstXI, EcoRI, BamHI, SacI, KpnI, and HindIII cloning site, N-terminal peptide purified with ProBond resin and cleaved with enterokinase; Invitrogen), to mention just two, can be employed according to the present invention.

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

A preferred vector for the present invention is a Moloney murine leukemia virus derived vector.

Vectors are introduced into the desired host cells by methods known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., Wu et al., 1992, J. Biol. Chem. 267: 963-967; Wu and Wu, 1988, J. Biol. Chem. 263: 14621-14624; Hartmut et al., Canadian Patent Application No. 2,012,311, filed Mar. 15, 1990).

A preferred method of use for the invention is to treat mammals against Eph positive tumors. As described above various nucleic acid and amino acid sequences can be used to achieve this. A therapeutic composition as used herein, can include e.g. any of the above viruses or vectors containing the entire nucleic acid sequence of the Eph molecule, ephrin molecules, variants and fragments thereof; modified nucleic acid sequences of the Eph molecule; ephrin molecule, the entire amino acid sequence of the Eph molecule, ephrin molecule, or fragments thereof; modified amino acid fragments of the Eph molecule, ephrin molecule, or any peptides embodied in the invention.

The therapeutic composition may be introduced in a suitable carrier. For example, sterile saline solution or sterile phosphate buffered saline.

Another preferred method is using the above-described vectors, or other vectors well known in the art, for introducing vectors into cells or tissues which are equally suitable for use in vivo, in vitro, and ex vivo. For ex vivo therapy, vectors may-be introduced into stem cells taken from the patient and clonally propagated for autologous transplant back into that same patient. Macrophages also can be employed. Delivery by transfection, by liposome injections, or by polycationic amino polymers may be achieved using methods which are well known in the art (see, e.g., Goldman et al., 1997, Nature Biotechnology 15: 462-466).

Any of the therapeutic methods described above may be applied to any subject in need of such therapy, including, for example, mammals such as livestock such as sheep, goats, cattle and horses; pets such as dogs, cats and rabbits; preferably, primates such as monkeys; and, most preferably, humans.

Administration of Compositions to Animals

For targeting a tumor cell in situ, the compositions described above may be administered to animals including human beings in any suitable formulation. For example, compositions for targeting a tumor cell may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions of the invention may be administered to animals by any conventional technique. The compositions may be administered directly to a target site by, for example, surgical delivery to an internal or external target site, or by catheter to a site accessible by a blood vessel. Other methods of delivery, e.g., liposomal delivery or diffusion from a device impregnated with the composition, are known in the art. The compositions may be administered in a single bolus, multiple injections, or by continuous infusion (e.g., intravenously). For parenteral administration, the compositions are preferably formulated in a sterilized pyrogen-free form.

Formulations

While it is possible for an antibody or fragment thereof to be administered alone, it is preferable to present it as a pharmaceutical formulation. The active ingredient may comprise, for topical administration, from 0.001% to 10% w/w, e.g., from 1% to 2% by weight of the formulation, although it may comprise as much as 10% w/w but preferably not in excess of 5% w/w and more preferably from 0.1% to 1% w/w of the formulation. The topical fonrulations of the present invention, comprise an active ingredient together with one or more acceptable carrier(s) therefor and optionally any other therapeutic ingredients(s). The carrier(s) must be “acceptable” in the sense of being compatible with the other ingredients of the fonmulation and not deleterious to the recipient thereof.

Formulations suitable for topical administration include liquid or semi-liquid preparations suitable for penetration through the skin to the site of where treatment is required, such as liniments, lotions, creams, ointments or pastes, and drops suitable for administration to the eye, ear, or nose. Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified and sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogels. The formulation may incorporate any suitable surface active agent such as an anionic, cationic or non-ionic surface active such as sorbitan esters or polyoxyethylene derivatives thereof. Suspending agents such as natural gums, cellulose derivatives or inorganic materials such as silicaceous silicas, and other ingredients such as lanolin, may also be included.

Kits

In yet another aspect, the invention provides kits for aiding a diagnosis of tumor, tumor stage and the like, wherein the kits can be used to detect the markers of the present invention. For example, the kits can be used to detect any one or more of the markers described herein, which markers are differentially present in samples of a patient and normal subjects. The kits of the invention have many applications. For example, the kits can be used to differentiate if a subject has GBM versus, for example, other tumors, or has a negative diagnosis, thus aiding neuronal injury diagnosis. In another example, the kits can be used to identify compounds that modulate expression of one or more of the markers in in vitro or in vivo animal models to determine the effects of treatment. In another example, the kit provides a composition or panel of biomarkers. Exemplary EPH biomarkers include the EphA1, EphA2, EphA3, EphA4, EphA5, EphA6, EphA7, EphA8, EphB1, EphB2, EphB3, EphB4 and EphB5, eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk and nuk receptors. Ligands include, but not limited to, ephrin-A1, ephrin-A2, ephrin-A3, ephrin-A4, ephrin-A5, ephrin-B1, ephrin-B2 and ephrin-B3.

In one embodiment, a kit comprises (a) an antibody that specifically binds to a marker; and (b) a detection reagent. Such kits can be prepared from the materials described above, and the previous discussion regarding the materials (e.g., antibodies, detection reagents, immobilized supports, etc.) is fully applicable to this section and will not be repeated. Optionally, the kit may further comprise pre-fractionation spin columns. In some embodiments, the kit may further comprise instructions for suitable operation parameters in the form of a label or a separate insert.

In an additional embodiment, the invention includes a diagnostic kit for use in screening serum containing antigens of the polypeptide of the invention. The diagnostic kit includes a substantially isolated antibody specifically immunoreactive with polypeptide or polynucleotide antigens, and means for detecting the binding of the polynucleotide or polypeptide antigen to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, test serum is reacted with a solid phase reagent having a surface-bound antigen obtained by the methods of the present invention. After binding with specific antigen antibody to the reagent and removing unbound serum components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme which is detected by incubating the solid phase in the presence of a suitable fluorometric, luminescent or colorimetric substrate (Sigma, St. Louis, Mo.).

The solid surface reagent in the above assay is prepared by known techniques for attaching protein material to solid support material, such as polymeric beads, dip sticks, 96-well plate or filter material. These attachment methods generally include non-specific adsorption of the protein to the Support or covalent attachment of the protein, typically through a free amine croup, to a chemically reactive group on the solid support, such as an activated carboxyl, hydroxyl, or aldehyde group. Alternatively, streptavidin coated plates can be used in conjunction with biotinylated antigen(s).

Optionally, the kit may further comprise a standard or control information so that the test sample can be compared with the control information standard to determine if the test amount of a marker detected in a sample is a diagnostic amount consistent with a diagnosis of cancer, and/or effect of treatment on the patient.

In another embodiment, a kit comprises: (a) a substrate comprising an adsorbent thereon, wherein the adsorbent is suitable for binding a marker, and (b) instructions to detect the marker or markers by contacting a sample with the adsorbent and detecting the marker or markers retained by the adsorbent. In some embodiments, the kit may comprise an eluant (as an alternative or in combination with instructions) or instructions for making an eluant, wherein the combination of the adsorbent and the eluant allows detection of the markers using gas phase ion spectrometry. Such kits can be prepared from the materials described above, and the previous discussion of these materials (e.g., probe substrates, adsorbents, washing solutions, etc.) is fully applicable to this section and will not be repeated.

In another embodiment, the kit may comprise a first substrate comprising an adsorbent thereon (e.g., a particle functionalized with an adsorbent) and a second substrate onto which the first substrate can be positioned to form a probe which is removably insertable into a gas phase ion spectrometer. In other embodiments, the kit may comprise a single substrate which is in the form of a removably insertable probe with adsorbents on the substrate. In yet another embodiment, the kit may further comprise a pre-fractionation spin column (e.g., Cibacron blue agarose column, anti-HSA agarose column, size exclusion column, Q-anion exchange spin column, single stranded DNA column, lectin column, etc.).

Optionally, the kit can further comprise instructions for suitable operational parameters in the form of a label or a separate insert. For example, the kit may have standard instructions informing a consumer how to wash the probe after a sample is contacted on the probe. In another example, the kit may have instructions for pre-fractionating a sample to reduce complexity of proteins in the sample. In another example, the kit may have instructions for automating the fractionation or other processes.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

### EXAMPLES

Materials and Methods

Cell Lines and Tissues

Cell lines, such as U-87 MG, SNB-19, U-251 MG, and A-172 were obtained from American Type Culture Collection (Manassas, Va.). G-48a cells were primary high-grade astrocytoma (HGA) cultures isolated in this laboratory. 6B53-11 cells were obtained as a gift from David James of the Mayo Clinic (Rochester, Minn.). All cell lines were grown in the appropriate media.

GBM tumors and normal brain tissue were obtained from the operating room and frozen immediately. Ten-micron sections of GBM were thaw mounted onto slides, which were stored at −80° C. until assayed. Sections were thawed and subsequently fixed for 10 min in acetone at −20° C.

Immunohistochemistry and Immunocytochemistry

GBM cell lines and human explant cells were grown overnight on sterile glass slides in the appropriate media. Slides were washed twice in phosphate-buffered saline (PBS) and fixed for 2 min in acetone at −20° C. Slides were then washed twice in PBS and either used immediately or stored at −80° C. until use.

Rabbit polyclonal EphA2 antibody (1:100) was purchased from Santa Cruz Biotechnology (Santa Cruz, Calif.). Mouse monoclonal EphA2 clone D7 antibody was purchased from Sigma. Slides were washed twice in PBS and blocked for 1 hour in 10% normal goat serum (NGS) at room temperature. Primary antibodies were diluted in 1.5% NGS and incubated overnight at 4° C. Slides were washed three times in PBS, 5 min each. Secondary antibodies were goat anti-rabbit rhodamine (1:200), donkey anti-rabbit rhodamine (1:200) (both from Jackson ImmunoResearch Laboratories, Inc., West Grove, Pa.) or goat anti-mouse IgG Oregon Green (1:200) (Molecular Probes, Eugene, Oreg.). Slides were washed three times for 5 min each in PBS and mounted with Gel-Mount (Biomeda Corp., Foster City, Calif.). Slides were counterstained with Hoescht No.33258 Nuclear Counterstain (DAPI).

Photomicrographs were taken at 63× magnification with an oil immersion lens in all cases with an Axiovision camera. Background was normalized to the samples without primary antibody. Images were processed with Adobe Photoshop 5.0LE.

Western Blots

Cell lysates were prepared from subconfluent cultures. Cells were washed with PBS and lysed in RIPA buffer. Non-malignant brain and GBM tumor tissue were minced into small pieces while frozen and thawed in RIPA buffer with Mammalian Protease Inhibitor Cocktail. Lysates were passed through an 18-gauge needle to shear the DNA. PMSF was added and incubated on ice for 30-60 min. Non-soluble debris was pelleted at 10,000 rpm for 10 min and the supernatant was collected and stored at −80° C. until use. Normal human brain lysates were also purchased from Chemicon International, Inc. (Temecula, Calif.) and Clontech Laboratories, Inc. (Palo Alto, Calif.). Lysates were run on 10% SDS-PAGE. Proteins were transferred to PVDF membrane and blocked for at least 1 hour with blotto (5% dry milk made up in PBS plus 0.05% Tween-20). Membranes were incubated with primary antibody diluted in blotto overnight at 4° C. while shaking. Primary antibodies included anti-mouse EphA2 (1:10000 and 1:5000) and anti-mouse β-actin (1:50,000) from Sigma and anti-rabbit EphA2 (1:100) from Santa Cruz Biotechnologies. Following three 5 min washes in PBS/0.05% Tween-20, membranes were incubated with secondary antibody conjugated with horseradish peroxidase (goat anti-mouse IgG or goat anti-rabbit IgG) at a dilution of 1:5000 in blotto for 1 hour. Membranes were washed three times, 5 min each in PBS/0.05% Tween-20 and detection was performed using the ECL plus Western Blotting Detection System (Amersham Biosciences, UK). Membranes were exposed to autoradiographic film X-OMAT AR for various times.

### Example 1

### EphA2 is Overexpressed in GBM Cells in Culture

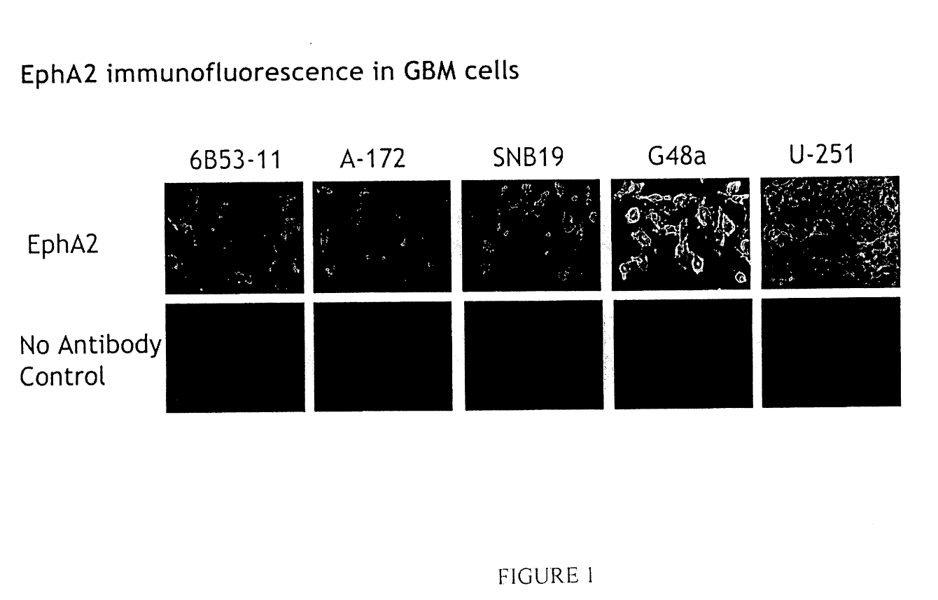

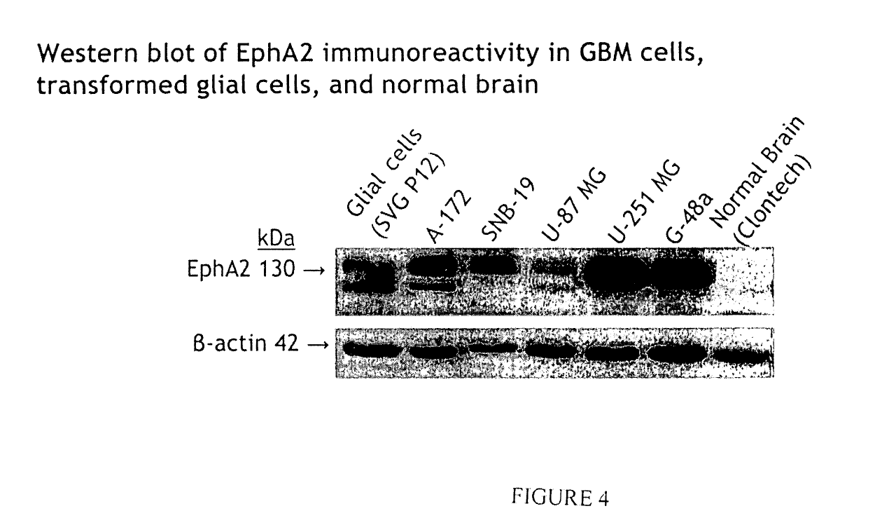

Analysis of EphA2 in GBM cells showed that this receptor is overexpressed at the protein level. Specific staining for EphA2 was observed by immunofluorescence using both polyclonal (cells red in color) and monoclonal (cells green in color) EphA2 antibodies (FIG. 1). Notably, EphA2 staining of U-251 cells showed a distinct honeycomb pattern that is indicative of the expected membrane-localized expression. Also, Western blot analysis of lysates from five different established GBM cell lines and one transformed glial cell line demonstrated elevated levels of EphA2 protein (FIG. 4). Interestingly, a normal human brain protein medley purchased from Clontech as well as tissue from the frontal lobe of a normal human showed little to no expression of EphA2 (FIG. 4).

### Example 2

### EphA2 is Abundant in Human GBM Tumors

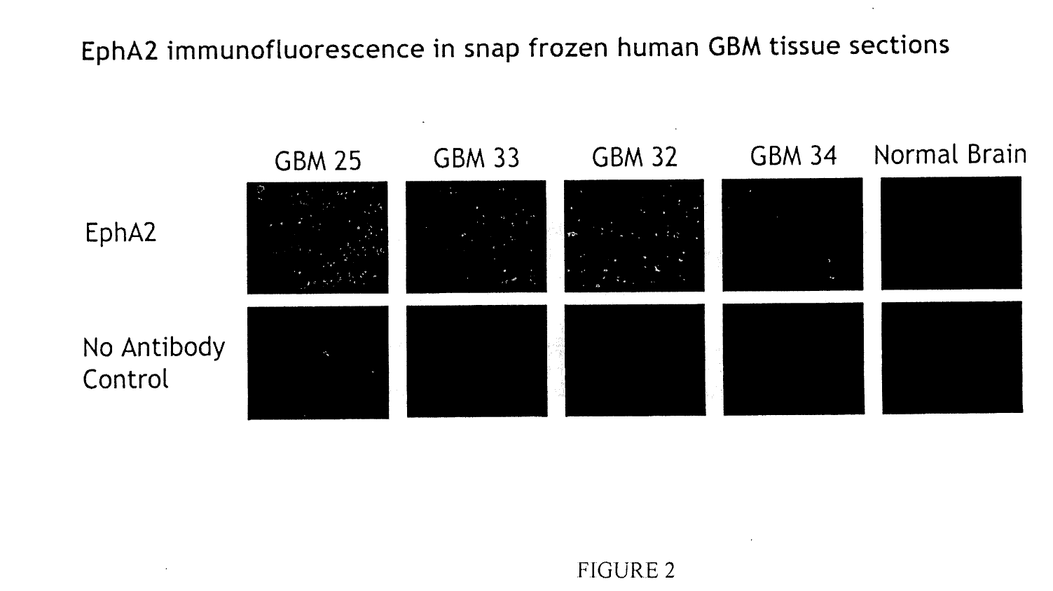

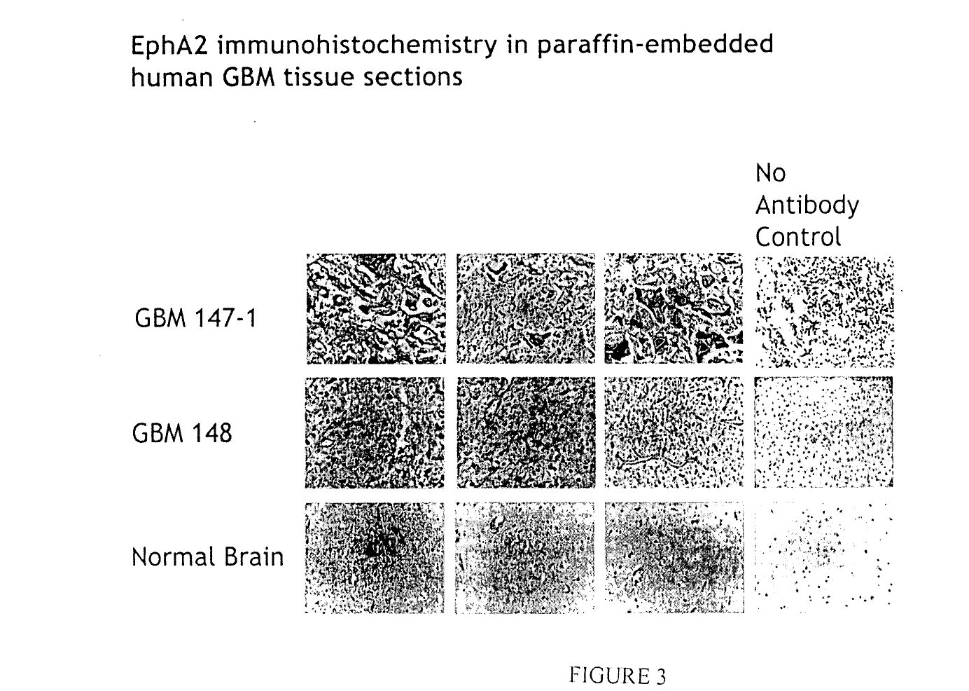

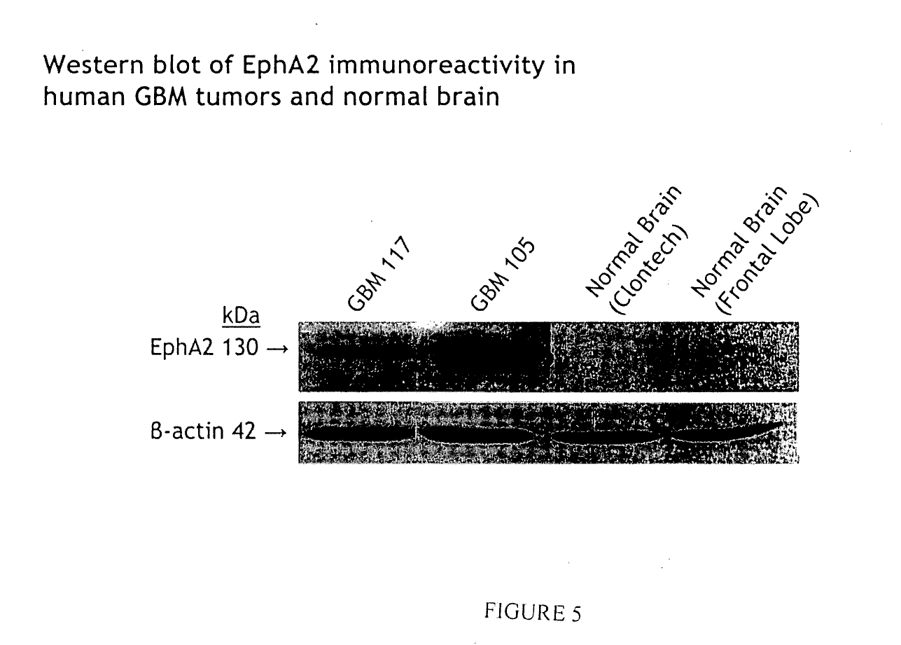

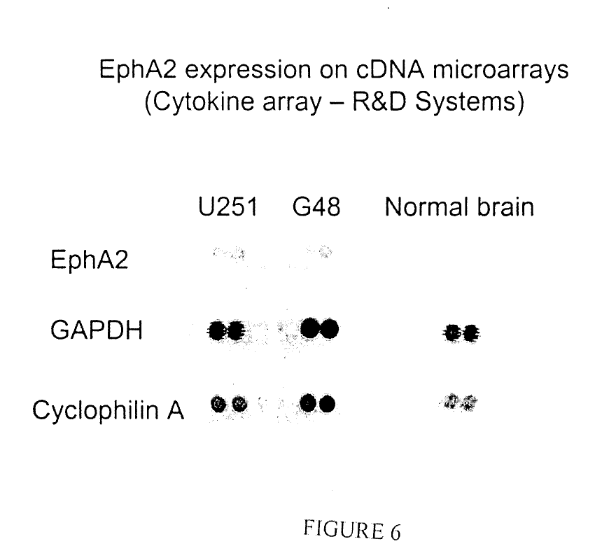

Immunofluorescence of EphA2 in frozen GBM sections revealed that the receptor is expressed at significant levels in human GBM tumors (FIG. 2). The staining of paraffin-embedded human GBM tissue and normal brain illustrated that EphA2 is abundant in GBM, but that it is nearly absent in normal brain (FIG. 3). EphA2 protein is also present at high levels in GBM tumors as shown by Western blot analysis (FIG. 5), but not in normal human brain.

We have documented specific, differential expression of EphA2 in both GBM cells and human GBM tumors compared to normal brain. Therefore, EphA2 serves as a useful molecular marker for GBM in such areas as diagnosis and prognosis. In addition, EphA2 can be used in the development of new therapeutics for GBM, such as molecularly targeted drug delivery.

### Example 3

### EphA2 as a Novel Molecular Marker and Target in Glioblastoma Multiforme

Glioblastoma multiforme (GBM) is an extremely invasive, well-vascularized tumor believed to be of astroglial origin. It is the most prevalent and lethal of all primary malignant brain tumors, with a median survival rate of about 12 months. Despite the standard treatment of surgical resection of the tumor followed by radiation and/or chemotherapy, the survival rate has increased only slightly over the past three decades. It is clear that novel therapies are needed to improve the prognosis and quality of life of patients with GBM. Molecular markers that are either found specifically on tumor cells or are highly overexpressed on malignant cells and nearly absent on normal cells are attractive therapeutic targets for approaches such as targeted drug delivery. Along these lines, we previously identified a receptor for interleukin 13 (IL-13), ILRα2, which is a brain tumor-associated cancer/testis tumor antigen (CTA) and is a very attractive therapeutic target.

The Eph receptors comprise the largest family of tyrosine kinase receptors, a group of transmembrane proteins that are crucial in mediating important signal transduction pathways in cells such as those controlling growth, migration, and differentiation. The fourteen members of the Eph receptors are divided into A and B classes based on the homology of their extracellular domains, which typically include a globular amino-terminal ligand-binding domain followed by a cysteine-rich domain and two fibronectin type-III repeats. The C-terminal intracellular domain that is conserved among all Eph receptors contains two tyrosine residues involved in the auto-phosphorylation activity of the receptor and is followed by a tyrosine kinase catalytic domain. The phosphorylation of both the membrane proximal tyrosines as well as those in the catalytic region controls Eph receptor biological activity.

The Eph receptors are unique among the tyrosine kinase receptors in that their endogenous ligands, the ephrins, are bound to the surface of neighboring cells. The ephrins are a family of cell-surface anchored proteins of two classes based on how they are attached to the plasma membrane. EphrinA ligands are attached via a glycosylphosphatidylinositol (GPI) linkage, whereas ephrinB ligands possess a transmembrane sequence with an intracellular domain that mediates attachment to the cell membrane. All of the ephrins interact with specific Eph receptors, although there is promiscuity as some ephrins bind to more than one Eph receptor.

Eph receptors and ephrin ligands display specific patterns of expression during development. They have been implicated in the complex process of establishing boundaries between populations of cells during the formation of the body plan. In neural development, Eph receptors and their ligands have been shown to play an important role in axon guiding through the mediation of contact-dependent processes between cells. EphA2 is present in the nervous system during embryonic development, but unlike most other Eph receptors, it is also expressed on the surface of proliferating adult epithelial cells. However, this expression is at a relatively low level and most commonly limited to the skin, intestine, lung and ovary. Notably, in these cells, the receptor is localized to points of cell-cell contact and bound by its ligand, ephrinA1.

EphA2 is significantly overexpressed in several human epithelial malignancies, such as breast, colon , ovarian, prostate and pancreatic carcinomas. This elevation in the level of EphA2 is thought to be due in part to a decrease in the amount of ligand-mediated receptor degradation. For example, the unstable cell-cell contacts within cancer tissue may hinder the ability of EphA2 to interact with ephrinA1 on neighboring cells. As a result, receptor activation as well as the subsequent degradation of EphA2 are markedly decreased. Interestingly, this produces a situation in transformed epithelial cells in which EphA2 is significantly less activated, and simultaneously highly overexpressed. The activation of EphA2 negatively regulates integrin-mediated adhesion, cell spreading and migration. Activated EphA2 suppresses the function of integrins and directly cause the dephosphorylation of and subsequent dissociation from focal adhesion kinase. Furthermore, EphA2 activation inhibits cell growth and proliferation and decreases cell-extracellular matrix contacts. Hence, when EphA2 is present in its inactive state, the cells on which it is expressed may have a tendency to be more motile, invasive, and faster-growing. It is thus one embodiment of the invention to correlate the overexpression of inactive EphA2, both in vitro and in vivo with invasiveness and aggressive phenotypes of the aforementioned cancers.

EphA2 also plays an important role in angiogenesis and tumor neovascularization through association with its endogenous ligand, ephrinA1. EphrinA1 was originally described as a TNF-α inducible endothelial gene product, and a role for ephrinA1 as an important factor in angiogenesis has been proposed (Pandey A, Shao H, Marks R M, Polverini P J, and Dixit V M. Science 268: 567-569, 1995). In contrast to the situation in epithelial cells, the failure of ephrinA1 to activate EphA2 in normal endothelial cells inhibits vascular endothelial growth factor-induced angiogenesis.

Cell Lines and Tissues: Cell lines derived from human glioblastoma multiforme (U-87 MG, U-251 MG, DBTRG-05 MG, and A-172 MG), human malignant glioma (H4) and normal human endothelial (HUVEC) were obtained from American Type Culture Collection (Manassas, Va.). G48a cells were primary GBM human explant cell cultures isolated in this laboratory. 6B53-11 GBM cells were obtained as a generous gift from Dr. David James of the Mayo Clinic (Rochester, Minn.). The 6B53-11, A-172 MG, and H4 malignant glioma cells were grown in Dulbecco's Modified Eagle's Medium (D-MEM) with 10% Fetal Calf Serum (FCS) (Life Technologies, Rockville, Md.). The U-87 MG cells were grown in Earle's Minimum Essential Medium (MEM), 10% FCS, 0.1 mM Non-essential Amino Acids (NEAA), 2 mM Glutamine (Life Technologies), and 100 μg/mL Sodium Pyruvate. G48a cells were grown in RPMI-1640 (Life Technologies), 10% FCS, 100 μg/mL Sodium Pyruvate, 100 μg/mL L-Cysteine (Life Technologies), 20 μg/mL L-Proline (Sigma, St. Louis, Mo.), 1× HT Supplement consisting of 0.1 μM Sodium Hypoxanthine and 0.016 μM Thymidine, 5 units/mL Penicillin G and 5 units/mL Streptomycin sulfate (Life Technologies). TNF-α was obtained from R&D Systems (Minneapolis, Minn.). Tissue samples from human GBM and normal brain were obtained from the operating room, formalin-fixed, or frozen immediately and stored at −80° C. Ten-micron sections of GBM were thaw-mounted onto slides, which were stored at −80° C. until assayed.

Western Blot: Cell lysates were prepared from sub-confluent cultures. Cells were washed with phosphate-buffered saline (PBS) and lysed in RIPA buffer (PBS, 0.5% sodium deoxycholate, 0.1% SDS and 0.5% Igepal) containing Mammalian Protease Inhibitor Cocktail (Sigma). Non-malignant brain and pathologist-verified GBM tumor tissue was minced into small pieces while frozen and homogenized in RIPA buffer with Mammalian Protease Inhibitor Cocktail (Sigma). Lysates were passed through an 18-gauge needle to shear the DNA and were incubated on ice for 60 minutes. Non-soluble debris was pelleted at 10,000 rpm for 10 min and the supernatant was collected and stored at −80° C. until use. Normal human brain lysates were purchased from Chemicon International, Inc. (Temecula, Calif.) and Clontech Laboratories, Inc. (Palo Alto, Calif.). Lysates were separated by SDS-PAGE using 10% or 15% acrylamide or with 4-15% Tris-HCl gradient gels (Bio-Rad Laboratories). Proteins were then transferred to a PVDF membrane (Pierce, Rockford, Ill.) and blocked for at least 1 hour with blotto (5% milk in PBS/0.05% Tween-20). Membranes were incubated with primary antibody diluted in blotto overnight at 4° C. while shaking. Rabbit polyclonal EphA2 (1:100) and ephrinA1 (1:150) antibodies were purchased from Santa Cruz Biotechnology (Santa Cruz, Calif.). Mouse monoclonal EphA2 clone D7 (1:500), phosphotyrosine clone PY20 (1:1000) and β-actin (1:50,000) antibodies were purchased from Sigma (St. Louis, Mo.). Following three 5 min washes in PBS/0.05% Tween-20, membranes were incubated with secondary antibody conjugated with horseradish peroxidase (goat anti-mouse IgG or goat anti-rabbit IgG) at a dilution of 1:5000 in blotto for 1 hour. Membranes were washed three times, 5 min each in PBS/0.05% Tween-20 and detection was performed using the ECL plus Western Blotting Detection System (Amersham Biosciences, UK). Membranes were exposed to autoradiographic film X-OMAT AR for various times. Films were scanned at 600× dpi and images compiled using Jasc Paint Shop Pro v 6.0.

Immunofluorescence and Immunohistochemistry: For immunofluorescence, GBM cell lines and human explant cells were grown overnight on sterile glass slides in the appropriate media. Slides were washed twice in phosphate-buffered saline (PBS) and fixed for 2 min in acetone at −20° C. Slides were then washed twice in PBS and either used immediately or stored at −80° C. until use. Frozen sections were thawed and subsequently fixed for 10 min in acetone at −20° C. Slides were washed twice in PBS and blocked for 1 hour in 10% normal goat serum (NGS) at room temperature. Primary antibodies EphA2 polyclonal (1:200) or monoclonal (1:1000) or EphrinA1 polyclonal (1:200) were diluted in 1.5% NGS and incubated overnight at 4° C. No antibody control slides were incubated with 1.5% NGS. Slides were washed twice in PBS, 5 min each and incubated with secondary antibody for 45 minutes at room temperature. Secondary antibodies included goat anti-rabbit rhodamine (1:200), donkey anti-rabbit rhodamine (1:200) (both from Jackson ImmunoResearch Laboratories, Inc., West Grove, Pa.) or goat anti-mouse IgG Oregon Green (1:200) (Molecular Probes, Eugene, Oreg.). Slides were counterstained with Hoechst No. 33258 Nuclear Counterstain (DAPI). Slides were washed two times for 5 min each in PBS and mounted with Gel-Mount (Biomedia Corp., Foster City, Calif.).

For immunohistochemistry (IHC), tumor specimens were fixed in buffered formalin and embedded in paraffin. 5 μm sections were cut and mounted on chrom-alum slides. Tissue microarrays were obtained from Cybrdi, Inc. (Gaithersburg, Md.). After completely dry, slides were baked at 65° C. until the paraffin was melted. Slides were de-paraffinized in xylene and re-hydrated through alcohol. Antigen retrieval was performed with 10 mM sodium citrate buffer, pH 6.0, by microwaving twice on medium for 5 min each. Once cooled, endogenous peroxidase activity was quenched by incubating slides for 30 min in a peroxide/methanol bath. Slides were washed with three changes of PBS over 5 min. Staining was performed using the SensiTek HRP Anti-Polyvalent kit (ScyTek Laboratories, Logan, Utah). Background staining was blocked with ScyTek Superblock for 5 min. Slides were washed with PBS and incubated with primary antibody (EphA2 polyclonal, 1:200 or EphrinA1 polyclonal, 1:200) made in PBS overnight at 4° C. No antibody control slides were incubated with PBS. Excess antibody was removed by washing with PBS. Slides were then blocked in diluted Superblock (1:10 in PBS) for 15 min and washed with PBS. Slides were incubated in ScyTek biotinylated secondary antibody for 15 min then washed with PBS. ScyTek Avidin-HRP was applied to the slides and allowed to incubate for 20 min. Slides were rinsed in distilled water. Visualization with ScyTek AEC/Chromagen was performed and allowed to proceed for 8-10 min. Slides were rinsed with tap water and counterstained in hematoxylin for 1 minute. Slides were given a final rinse in tap water and mounted with Crystal-Mount (Biomedia).

Photomicrographs were taken with a 63× magnification oil immersion lens in all cases with a Zeiss Axiovision camera. Background was normalized to the samples without primary antibody. Images were processed with Jasc Paint Shop Pro v6.01.

Anchorage Independent Growth: 2×10³ U-251 MG, DBTRG-05 MG, U-87 MG, or H4 cells were plated in 6-well plates in growth medium plus 0.35% Agar (Fisher), on a base layer of growth medium plus 0.5% Agar. Cells were supplemented with a recombinant mouse ephrinA1/Fc chimera (R&D Systems) at 0.001, 0.01, 0.1, 0.5, 1 μg/mL, or vehicle alone (each concentration point was performed in triplicate). EphrinA1 was replenished with fresh media 3 days after plating, and colonies were counted at low power after 14 days at. Clusters of colonies greater than 75 cells for DBTRG-05 MG and U-251 MG or 25 cells for U-87 MG and H4 (these latter two cell lines grow poorly in soft agar) were counted in ten random fields at low power; each experimental condition was performed in triplicate for every assay.

Invasion Assay: BD Biocoat™ Matrigel™ Invasion Chambers, control inserts, and wells (BD Biosciences, Bedford, Mass.) were rehydrated with 500 μL serum-free media at 37° C. for 2 hours. Cells were pre-treated for 1 hour with 0.01, 0.1, 0.5, or 1.0 μg/mL ephrinA1-Fc at 37° C., trypsinized, quenched with PBS plus 0.1% BSA, and counted. After removing rehydration media, 750 μL of media plus 5% FBS was added to each well of the 24-well plate, followed immediately by the addition of 4×10⁴ cells in 500 μL serum-free media plus the appropriate concentration of ephrinA1-Fc to each chamber and control insert. Plates were incubated 20-22 hours at 37° C. Non-invading cells were removed from the upper surface of the membrane by scrubbing the membrane with a cotton swab. Cells on the lower surface of the membrane were stained with the DIFF QUIK stain kit (IMEB, Inc., San Marcos, Calif.). Invading cells in 5 random fields were counted with a 40× lens for the three Matrigel inserts and control inserts used for each cell type and treatment condition (each was performed in triplicate), to obtain the mean number of cells migrating through the Matrigel membrane and the control insert membrane. Data is expressed as the percent invasion through the Matrigel Matrix membrane relative to migration through the control membrane.

Immunoprecipitation: Cell lysates were prepared from sub-confluent cultures. Cells were washed with PBS and lysed in RIPA buffer (PBS, 0.5% sodium deoxycholate, 0.1% SDS and 0.5% Igepal) containing Mammalian Protease Inhibitor Cocktail (Sigma) and 1 mM sodium vanadate. Approximately 500 μg of cell lysate was incubated with 5 μg of monoclonal EphA2 (clone D7) overnight at 4° C. 70 μL of a 50% PBS/bead slurry containing ˜35 μL of packed Protein G-Sepharose beads (Sigma, St. Louis, Mo.) were added and incubated for a minimum of 1 hour at 4° C. Beads were collected by centrifugation, washed three times with ice-cold RIPA buffer, and resuspended in 60 μL 3× SDS sample buffer (New England Biolabs). Samples were heated at 100° C. for 5 minutes. Supernatant was collected and stored at −20° C. until separated using SDS-PAGE for Western blotting.

EphA2 Receptor Phosphorylation: Sub-confluent cultures of U-251 MG cells were serum-starved overnight in 100 mm dishes. Cells were treated with a recombinant mouse ephrinA1/Fc chimera (R&D Systems) or mouse monoclonal IgG, isotype control (R&D Systems) for the indicated times. Cell lysates were prepared at the indicated times and used for immunoprecipitation with EphA2 followed by Western blotting for P-Tyr and, subsequently, EphA2 (see above).

Results

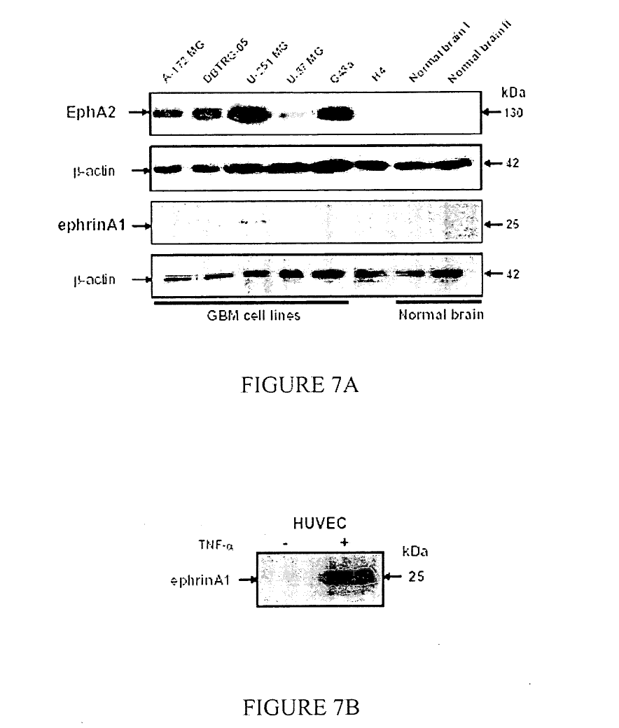

Expression of EphA2 and ephrinA1 in established human GBM cell lines: The level of EphA2 and ephrinA1 proteins in several GBM cell lines and normal brain was first determined by Western blotting. Five different GBM cell lines (A-172 MG, DBTRG-05 MG, U-251 MG, and G48a ) displayed highly elevated levels of EphA2 protein, which migrated as an immunoreactive band at the expected size of 130 kDa (FIG. 7A). The exception was U-87 MG cells, which expressed much less EphA2 than the other GBM cell lines tested (FIG. 7A). Normal brain protein medleys isolated from the frontal lobe of a normal brain (FIG. 1A, Normal brain I) as well as those obtained from Clontech (FIG. 7A, Normal brain II) and Chemicon demonstrated either a very faint immunoreactive band or the absence of any band at 130 kDa (FIG. 7A). Notably, a non-tumorigenic malignant glioma cell line, H4, displayed levels of immunoreactive EphA2 similar to that observed in normal brain (FIG. 7A).

Immunoblotting for the ephrinA1 ligand in GBM cell lines revealed strikingly different results than those observed for EphA2. There was no indication of an immunoreactive band migrating at the expected size of ephrinA1 (˜25 kDa) in most of the same cell lines in which EphA2 was abundantly overexpressed, such as A-172 MG, DBTRG-05 MG and G48a (FIG. 7A). In U-251 MG cells, however, a faint ephrinA1 immunoreactive band was observed (FIG. 7A). In addition, normal brain tissue possessed very low levels of immunoreactive ephrinA1, similar to the level of EphA2 observed in the same samples (FIG. 7A). Because ephrinA1 is a TNF-α inducible gene product, human umbilical vein endothelial cells (HUVEC) stimulated with TNF-α were included as a positive control for ephrinA1 detection in cell lysates (FIG. 7B).

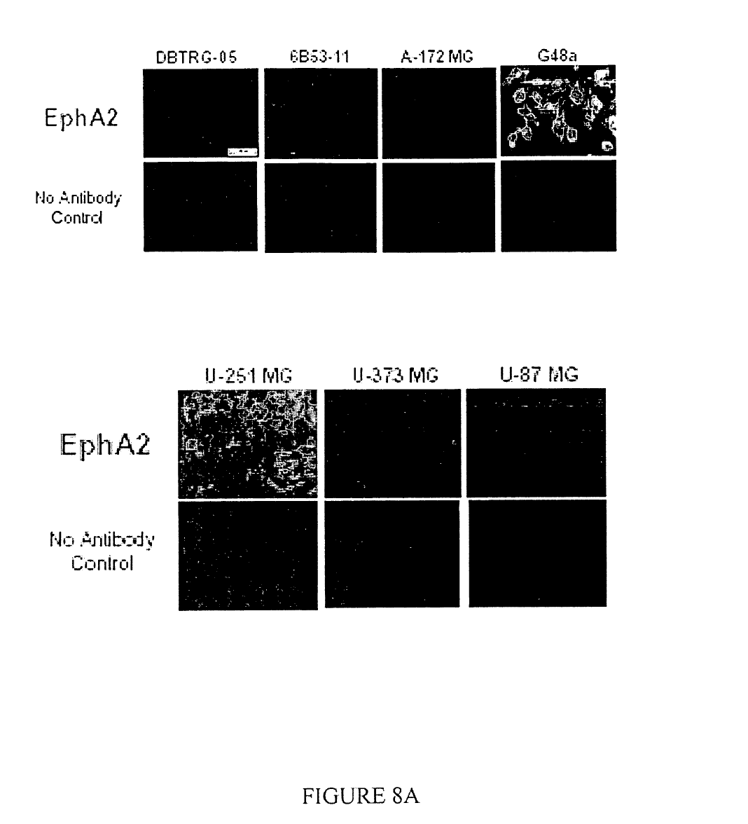

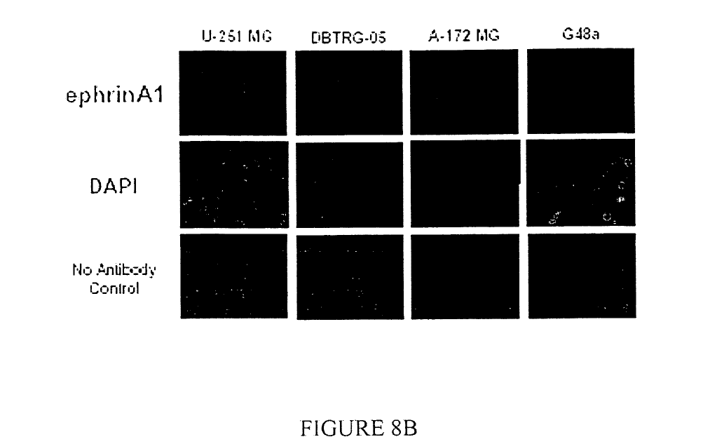

Next, immunofluorescence was performed to further investigate the expression as well as the localization of EphA2 and ephrinA1 in GBM cells. Abundant, specific staining for EphA2 was observed in all GBM cell lines examined using both monoclonal and polyclonal EphA2 antibodies (FIG. 8A). In confluent cells, a distinct, honeycomb pattern of staining was evident (FIG. 8A, U-251 MG cells), which is indicative of the expected membrane-localized expression of the receptor. Some cytoplasmic and perinuclear staining was also seen. In one GBM cell line, U-87 MG, the level of EphA2 immunofluorescence was observed to be visibly lower compared with all other GBM cells (FIG. 8A). This finding is consistent with the low level of immunoreactive EphA2 protein found in U-87 MG cells by Western blotting (FIG. 7A).

To further investigate the expression of EphA2 and ephrinA1 in GBM, immunofluorescence was performed for ephrinA1 in the same GBM cells that had been stained for EphA2. Very low levels of ephrinA1-specific staining was found, most of which was close to the detection limit of the assay (FIG. 8B: 6B53-11, U-373 MG, and U-87 MG cells).

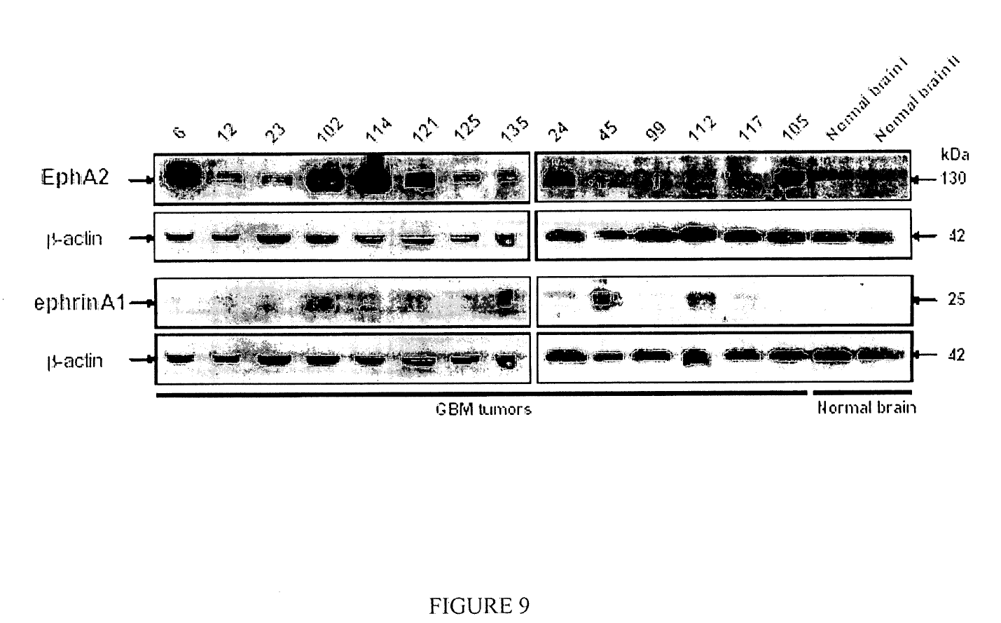

Expression of EphA2 and ephrinA1 in human GBM specimens: Next, the expression of the receptor and its ligand in GBM specimens and normal brain tissue was investigated by Western blotting and IHC. For Western blotting, whole tissue lysates were prepared from snap-frozen human GBM, from the frontal lobe of a normal brain, or commercially purchased. It was found that immunoreactive EphA2 was elevated above that in normal brain in 13 out of 14 GBM tumors examined, while 6 tumors exhibited large overexpression of EphA2 (FIG. 9). In 9 out of 13 samples in which EphA2 was elevated, immunoreactive ephrinA1 was present at markedly lower levels (FIG. 9, GBM 6, 12, 23, 114, 121, 125, 24, 117, and 105). The remaining 4 samples all displayed an increase in both EphA2 and ephrinA1immunoreactivity above that seen in normal tissue (FIG. 9, GBM 102, 135, 45, 112). Only one tumor, GBM 99, appeared to have low levels of both the receptor and the ligand (FIG. 9).

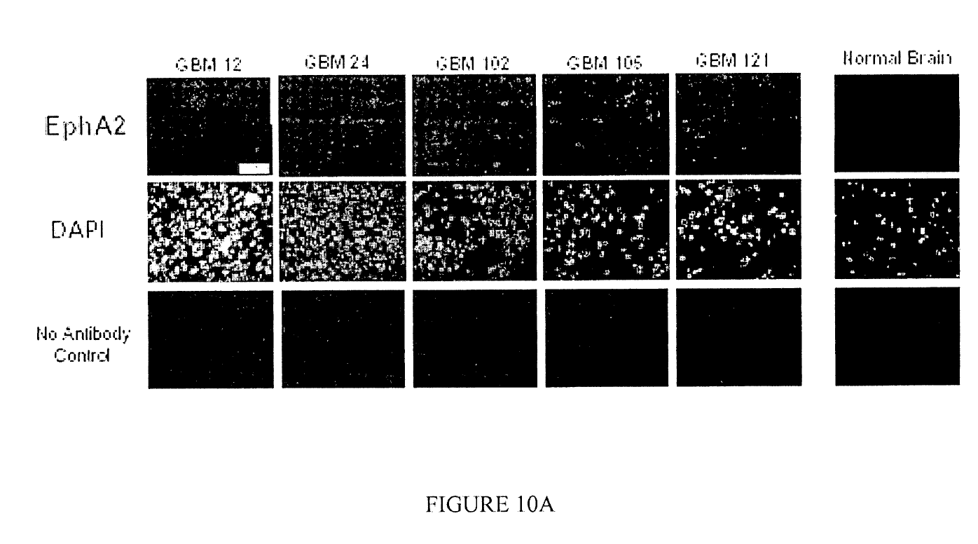

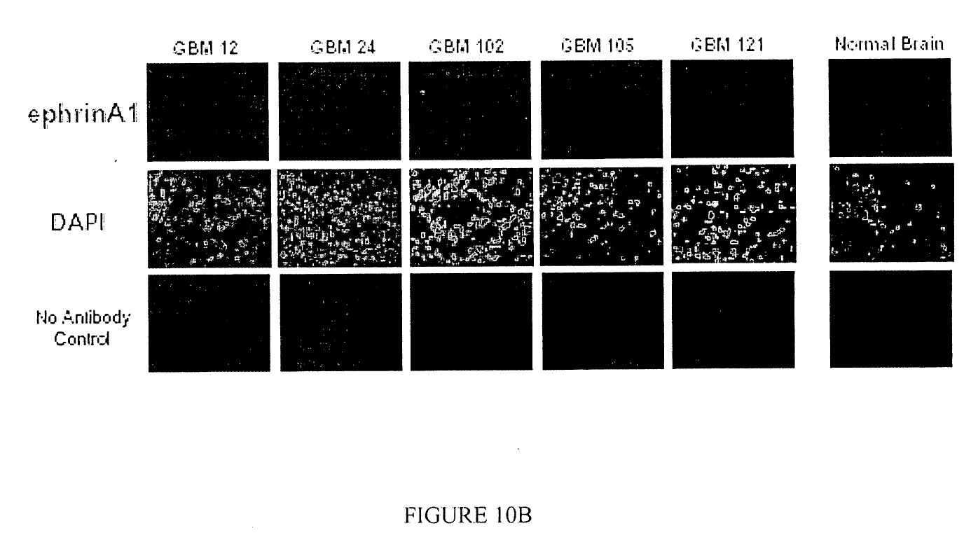

To further assess the expression of EphA2 and ephrinA1, immunofluorescence was performed on several frozen sections of human GBM tissue and non-malignant brain. These experiments revealed abundant staining for EphA2 in GBM and a very low level of detectable immunoreactive receptor in normal brain (FIG. 10A). Several different samples of normal brain were stained for EphA2 with consistently negative results. Immunofluorescent ephrinA1 was present at near background levels in all the same sections with the exception of GBM 102 (FIG. 10B), which also displayed an elevated level of ephrinA1 by Western blotting (FIG. 9). Notably, the level of EphA2 and ephrinA1 immunofluorescence observed in frozen sections was consistent with the amount of immunoreactive protein seen by Western blotting (FIGS. 9, 10A-10B).

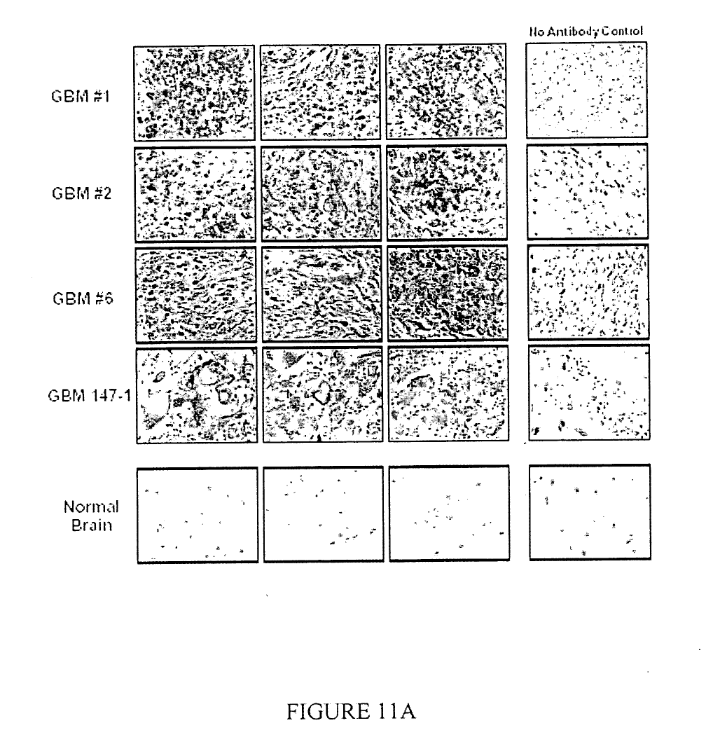

IHC on paraffin-embedded sections of human GBM revealed the same high degree of staining for EphA2 as was seen by Western blotting and immunofluorescence (FIG. 11A). In four different neuropathologist-verified GBM tumor specimens including giant cell GBM, dense, specific EphA2 staining throughout the sections, including glial cells and tumor vascular endothelial cells was observed (FIG. 11A). EphA2 staining was again near the detection limits of the assay in non-malignant brain tissue (FIG. 11A). Unlike the high degree of specific staining observed for EphA2, ephrinA1 was found at low levels throughout the GBM specimens (FIG. 11B). In non-malignant brain, ephrinA1 staining was observed, specifically and intensely localized to the endothelial cells of vessels (FIG. 11B), a phenomenon also seen to a small degree for EphA2 (FIG. 11A). Interestingly, this specific vascular endothelial cell localization of ephrinA1 in normal brain seemed less apparent in GBM (FIG. 11B). In addition, a tissue microarray composed of 61 paraffin-embedded sections of various brain tumors and normal brain revealed the strongest staining for EphA2 consistently among the high-grade (WHO Grade III-IV) astrocytomas; ephrinA1 staining of these sections revealed the same uniform low levels of the ligand.

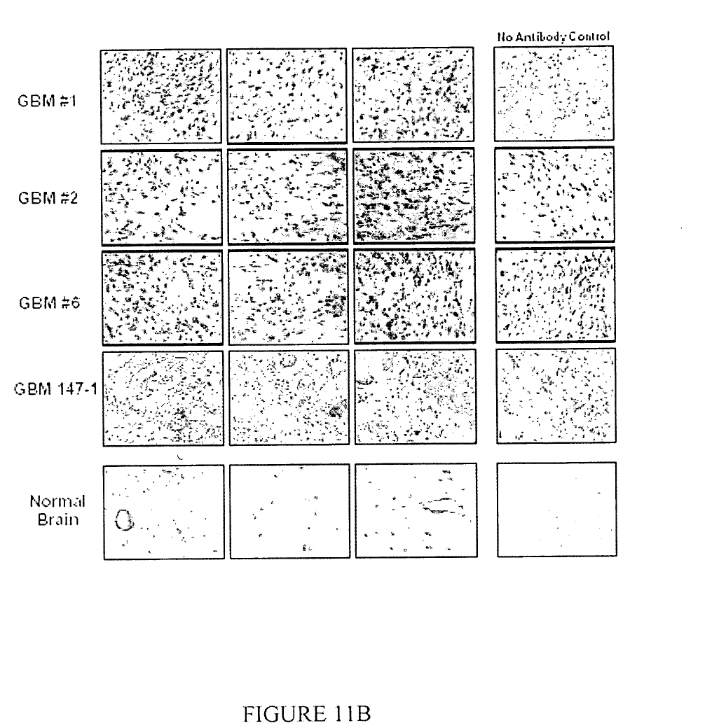

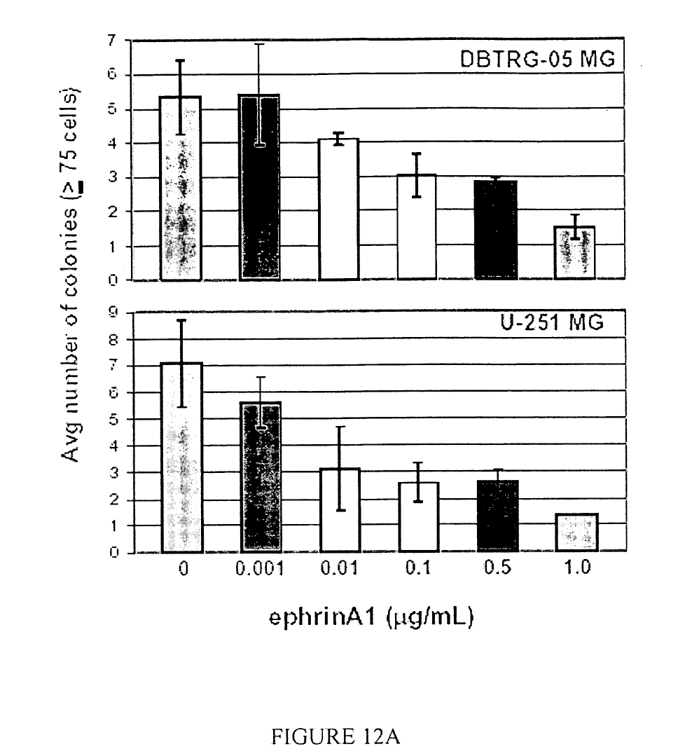

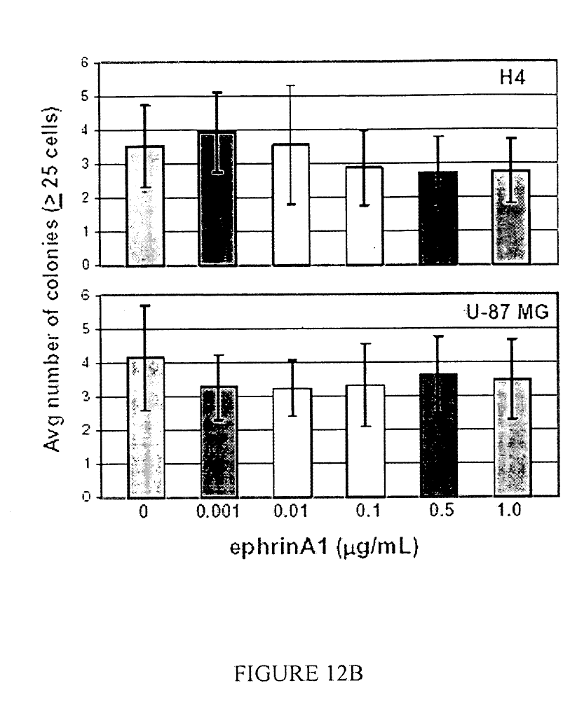

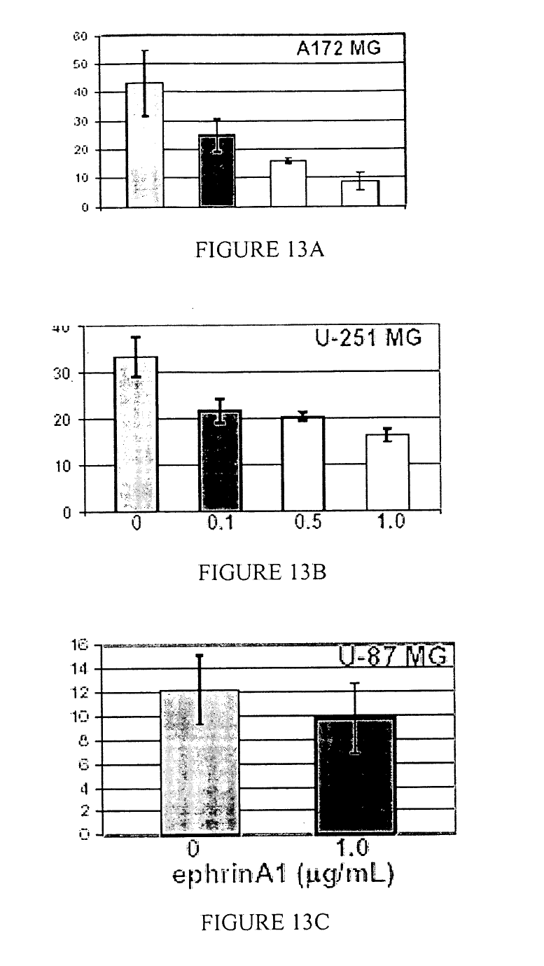

Effect of ephrinA1 on anchorage-independent growth of GBM cells: Due to the trend of differential expression of EphA2 and ephrinA1 observed in GBM cell lines and tumor tissue, we next investigated a possible functional relationship between the receptor and ligand in GBM. The effect of ephrinA1 on the anchorage-independent growth of high EphA2-expressing GBM cell lines was first examined by assessing the ability of U-251 MG and DBTRG-05 MG cells to form colonies in soft agar in the presence or absence of ephrinA1. A dose-dependent inhibitory effect of ephrinA1 on the anchorage-independent growth of the two cell lines, was found (FIG. 12A). Due to the fact that soluble ephrinA1 has been shown previously to activate EphA2 receptors in vitro, we suggest that ephrinA1-mediated activation of EphA2 has a negative effect on the anchorage-independent growth of GBM cells. To confirm that this phenomenon specifically involves ephrinA1-mediated activation of EphA2, the same experiment was performed using two cell lines that express low levels of EphA2: H4 and U-87 MG (FIG. 7A). We failed to observe any significant dose-dependent inhibition of anchorage-independent growth with increasing concentrations of ephrinA1 in both cell lines studied (FIG. 12B). In general, these cells formed smaller colonies in soft agar than the cells that highly overexpress EphA2.

Effect of ephrinA1 on GBM cells invasion: Another enhanced malignant feature of GBM cells that may result in part from a lack of EphA2 receptor activation is invasion. Therefore, the effect of exogenous ephrinA1 on the invasiveness of three different GBM cell lines, was investigated. The U-251 MG and A172 MG cells, high overexpressors of EphA2, exhibited a substantial dose-dependent decrease in invasiveness with increasing concentrations of ephrinA1 (FIG. 12). Treatment with 1.0 μg/mL of ephrinA1 caused an 80% decrease in invasion in A-172 MG cells and a 52% decrease in U-251 MG cells (FIG. 12). In contrast, the U-87 MG cells were less invasive than the high EphA2-expressing cell lines, and we observed only a 20% decrease in invasion from non-treated control to treatment with 1.0 μg/mL ephrinA1 (FIG. 12).

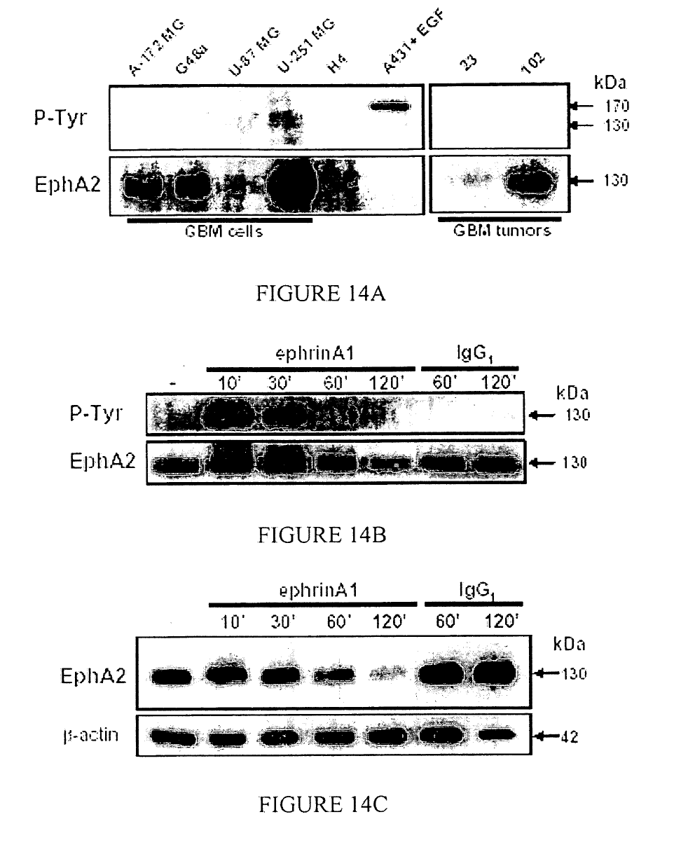

Activation status of the EphA2 receptor in GBM cells. The low levels of ephrinA1 expression in GBM suggested that the EphA2 receptors in these tumors are likely not activated, and thus are present in the non-tyrosine-phosphorylated state. To investigate this possibility, immunoprecipitation of the lysates from several GBM cell lines and tumors was performed using a monoclonal EphA2 antibody. As seen in FIG. 14A, little to no tyrosine phosphorylated EphA2 in GBM cell lines and tumors was detected, while immunoreactive EphA2 was readily observed. Notably, the amount of immunoreactive EphA2 immunoprecipitated from the two GBM tumors correlates directly with that found by Western blotting (FIG. 9). The lack of detectable tyrosine phosphorylation suggests that the EphA2 receptor is inactive, possibly due to lack of stimulation by ephrinA1 originating from neighboring cells. Upon treatment with ephrinA1, the levels of tyrosine phosphorylated EphA2 increased dramatically over time in U-251 MG cells, an indication of ephrinA1-mediated receptor activation (FIG. 14B). There was no increase in phosphorylated EphA2 with IgG₁-treated samples (FIG. 14B). EphrinA1-induced EphA2 phosphorylation was found to occur as early as 10 minutes following treatment and lasted for up to 60 minutes. The activation of the receptor seemed to be reversible, as phosphorylated EphA2 was no longer detected 2 hours following ephrinA1 stimulation (FIG. 14B). In addition, the levels of total immunoreactive EphA2 in ephrinA1-treated cell lysates began to decrease after 60 minutes of treatment (FIG. 14C).

We have demonstrated that EphA2 is abundantly and specifically overexpressed in GBM cell lines as well as human GBM tumor tissue. Important in the consideration of EphA2 as a potential molecular target for medical intervention, the expression of this receptor is detected at very low levels in normal brain. In addition, we documented that in cells and tumors which abundantly overexpress EphA2, ephrinA1 is, on average, present at much lower levels. Furthermore, our findings suggest the existence of a functional relationship between EphA2 and ephrin-A1 in tumors, such as GBM. We found that ephrinA1 activates EphA2 and elicits signals through tyrosine phosphorylation of the receptor that result in the negative regulation of oncogenic properties such as anchorage-independent growth and invasion.

To our knowledge, this is the first study investigating the presence and functional significance of the EphA2 receptor and its ligand, ephrinA1, in a human cancer of astroglial origin. In addition, another member of the Eph receptor family, EphB2, has been found to play a role in the migration and invasion of human glioma cells. The abundant overexpression of EphA2 in GBM may result from a decrease in the amount of ligand-induced receptor degradation. In normal tissues with stable cell-cell contacts, ephrinA1 binds and activates EphA2, causing the internalization of the receptor-ligand complex. Subsequently, EphA2 is degraded due to interaction with c-Cbl, and is found expressed at low levels in its activated state in normal adult epithelial tissue. In contrast, the overexpressed EphA2 in epithelial cancers is found predominately in the non-activated state, in which the tyrosines of the catalytic kinase domain remain un-phosphorylated. As our current results suggest, this situation holds true for malignant gliomas. We failed to detect any significant amount of EphA2 receptor tyrosine phosphorylation in both GBM cell lines and tumors, demonstrating that although overexpressed, EphA2 is present in the biologically inactive state. The very low levels of ephrinA1 in GBM cells and most tumor tissue may, at least partially, explain the lack of EphA2 receptor activation and resultant persistent over-expression. In addition, the gene expression for the receptor is also increased in GBM, which may contribute to the presence of elevated gene product in this disease.

The pattern of EphA2 and ephrinA1 differential expression was evident not only in cell lines, but also in GBM tumors. There were few exceptions, likely owing in part to the heterogeneous nature of tumors. One property of neoplastic transformation is a decrease in the amount of stable cell-cell contacts. Therefore, it is plausible that ephrinA1 is indeed present in some tumors, but can bind to EphA2 only in a short-lived manner that is insufficient to elicit receptor internalization and degradation. Alternatively, the majority of ephrinA1 may no longer exist in a form that is capable of successfully binding to and/or activating EphA2 in malignant tissue. For example, it is possible that ephrinA1 is cleaved from the surface of the cell and is present in the extracellular environment in a soluble, monomeric form, which does not effectively engage the EphA2 receptor. Both scenarios would promote the overexpression of EphA2.

EphA2 has been previously shown to be important in tumor neovascularization. Malignant gliomas are inherently highly vascular tumors. Angiogenic factors, such as vascular endothelial growth factors (VEGFs), are present at variable levels in the tumor microenvironment. The level of ephrinA1 present in a GBM tumor, therefore, may be in part related to tumor cell response to angiogenic factors. Alternatively, the presence of ephrinA1 in tumor tissue may be attributable to that expressed in the normal vasculature. Notably, we found ephrinA1 in normal brain tissue specifically localized to vascular endothelial cells, which was less apparent in GBM specimens. It is possible that different regions of a tumor, i.e., the core versus the invading edge have varying expression profiles of EphA2 and ephrinA1. EphA2, when activated by ephrinA1, signals through pathways involved in the negative regulation of cell growth, migration, proliferation, and invasion. We found that not only does ephrinA1 cause the phosphorylation of EphA2, but this change in the receptor status correlates with a decrease in anchorage-independent growth and invasion of GBM cells. Furthermore, these changes in cellular behavior caused by ephrinA1 appear to be related to the level of EphA2 expression, since they are not seen in low level EphA2-expressing cell lines. Hence, the EphA2/ephrinA1 system has the potential to play a highly significant role in GBM, as EphA2 overexpressed in an inactive form may allow unwarranted intracellular signals facilitating tumor progression and/or maintenance. Studies investigating these possibilities are ongoing in our laboratory.

We have shown that EphA2 is highly overexpressed and functionally important for the oncogenic properties of GBM. This work forms the basis for future studies investigating EphA2/ephrinA1 system as a target for the development of molecular-based interventions for high-grade gliomas.

### Example 4

### Identification of a Candidate Therapeutic Compound Binding to Ephrin Molecules

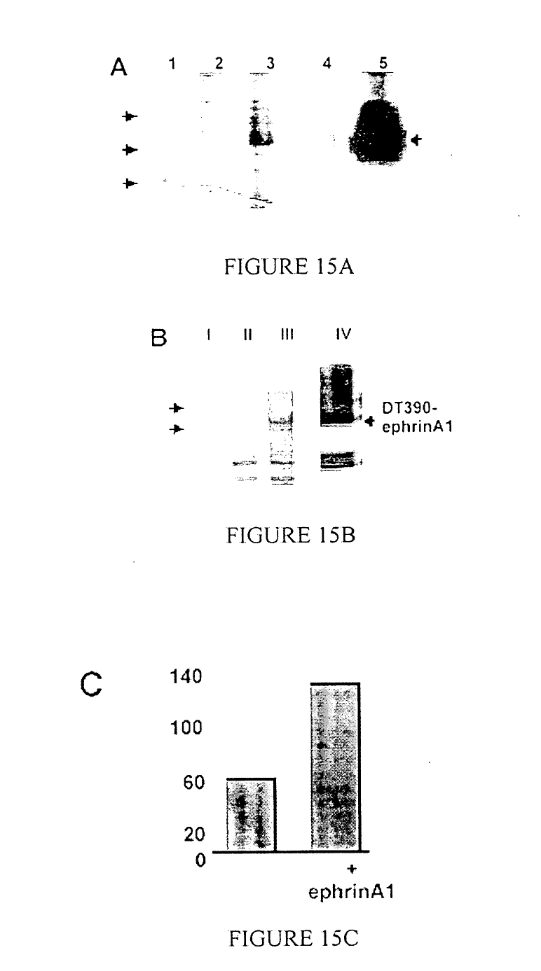

FIGS. 15A to 15C show EphrinA1 and DT390-ephrinA1 recombinant proteins expression in E. coli and partial purification of DT390-ephrinA1 cytotoxin. FIG. 15A: ephrin A1 is a major bacterial protein upon induction with IPTG. 1, molecular size markers; 2, pre-induced lysated of cells; 3, IPTG-induced production of ephrin A1 in bacteria (lanes 1 to 3 represent SDS-PAGE); 4, pre-induced lysated of cells; 5, IPTG-induced production of ephrin A1 in bacteria (lanes 4 to 5 represent Western blot). FIG. 15B: DT390-ephrin A1 is a major bacterial protein upon induction with IPTG. I, molecular size markers; II, pre-induced lysate of cells; III, IPTG-induced production of DT390-ephrin A1 in bacteria (lanes I to III represent SDS-PAGE); IV, Western blot using anti-ephrinA1 antibody of partially purified DT390-ephrinA1 cytotoxin. FIG. 15C: Cell proliferation assay in U-215 MG GBM cells using DT390-ephrinA1 (15 nM) in the absence or presence of an excess of ephrinA1-Fc.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition diagnostic for cancer comprising Eph receptors, Eph-related receptors, ephrin molecules, proteins, peptides, variants, fragments and derivatives thereof.

2. The composition of claim 1, wherein Eph receptors are selected from the group consisting of: EphA1, EphA2, EphA3, EphA4, EphA5, EphA6, EphA7, EphA8, EphB1, EphB2, EphB3, EphB4 and EphB5, eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk and nuk receptors and ephrin molecules comprise at least one of: ephrin A1, ephrin-A2, ephrin-A3, ephrin-A4, ephrin-A5, ephrin-B1, ephrin-B2 or ephrin-B3, variants and fragments thereof.

3. The composition of claim 1, wherein Eph receptors further comprise EphA2 receptors, proteins, peptides, variants, fragments and derivatives thereof.

4. A method for detection and diagnosis of cancer, and/or cancer related disorders comprising:
detecting at least one or more protein biomarkers in a subject sample, and; correlating the detection of one or more protein biomarkers with a diagnosis of cancer, wherein the correlation takes into account the detection of one or more biomarker in each diagnosis, as compared to normal subjects wherein the one or more protein markers are Eph receptors, EphA2 receptors, Eph-related receptors, ephrin molecules, fragments, and variants thereof; and,
correlating the detection of one or more protein biomarkers with a diagnosis of cancer, wherein the correlation takes into account the detection of one or more protein biomarkers in each diagnosis, as compared to normal subjects.

5. The method of claim 4, wherein the protein biomarkers are Eph receptors, EphA2 receptors, Eph-related receptors, fragments, and variants thereof.

6. The method of claim 4, wherein Eph receptors are selected from the group consisting of: EphA1, EphA2, EphA3, EphA4, EphA5, EphA6, EphA7, EphA8, EphB1, EphB2, EphB3, EphB4 and EphB5, eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk and nuk receptors, proteins, peptides, variants, fragments and derivatives thereof and ephrin molecules comprise any one of: ephrin A1, ephrin-A2, ephrin-A3, ephrin-A4, ephrin-A5, ephrin-B1, ephrin-B2 or ephrin-B3, variants and fragments thereof.

7. The method of claim 4, wherein Eph receptors further comprise EphA2 receptors, proteins, peptides, variants, fragments and derivatives thereof.

8. The method of claim 4, wherein amounts of Eph receptors, EphA2 receptors, ephrin proteins, peptides, variants, fragments and derivatives thereof are correlated with a cancer stage.

9. The method of claim 4, wherein one or more protein biomarkers are used to diagnose cancer.

10. The method of claim 4, wherein a plurality of protein biomarkers are used to diagnose cancer.

11. A method of identifying candidate therapeutic agents for treatment of tumors, comprising:
culturing an isolated cell expressing a receptor comprising any one of: EphA1, EphA2, EphA3, EphA4, EphA5, EphA6, EphA7, EphA8, EphB1, EphB2, EphB3, EphB4and EphB5, eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk or nuk receptors, proteins, peptides, variants, fragments and derivatives thereof; and,
administering a candidate therapeutic agent to the cultured cell; and,
correlating expression levels and phosphorylation of the receptor in the presence or absence of a candidate therapeutic agent as compared to a normal cell and a cell cultured in the presence of an ephrin molecule; thereby,
identifying candidate therapeutic agents that decrease Eph expression and activate Eph molecules, thereby,
identifying candidate therapeutic agents for treatment of tumors.

12. The method of claim 11, wherein activation of Eph molecules is determined by phosphorylation of tyrosine molecules as compared to Eph on a normal cell, Eph in the presence of its ephrin ligand and Eph in the presence of a candidate therapeutic agent.

13. The method of claim 11, wherein expression of Eph is compared to Eph in a normal cell, Eph+ cells in the presence of its ephrin ligand and Eph+ cells in the presence of a candidate therapeutic agent.

14. A composition for modulating Eph receptors comprising ephrin molecules, ephrin specific antibodies, peptides, variants and fragments thereof.

15. The composition of claim 14, wherein ephrin molecules further comprise ephrin A1, ephrin-A2, ephrin-A3, ephrin-A4, ephrin-A5, ephrin-B1, ephrin-B2 or ephrin-B3, variants and fragments thereof.

16. A method of treatment for subjects with EphA2 positive tumors comprising:
administering a therapeutically effective amount of a pharmaceutical composition comprising ephrins, ephrin A1, peptides, variants and fragments thereof to a patient in need of treatment; and,
modulating expression of Eph in tumors; thereby,
ameliorating EphA2 positive tumors.

17. The method of claim 16, wherein ephrin molecules modulate expression of Eph by binding the EphA2 receptor, activating the receptor an/or cellular internalization of the EphA2 receptor.

18. The method of claim 16, wherein activation of an Eph receptor is measured by detecting phosphorylation of EphA2 molecules.

19. The method of claim 16, wherein ephrin molecules are ephrin A1 proteins, peptides, variants, and fragments thereof.

20. The method of claim 16, wherein pharmaceutical compositions comprising ephrins are administered to a patient in conjunction with chemotherapeutic agents.

21. The method of claim 16, wherein the pharmaceutical composition comprises antibodies that specifically bind to EphA2.

22. The method of claim 16, wherein the pharmaceutical composition is administered to a patient by intravenous administration, intrasynovial administration, transdermal administration, intramuscular administration, subcutaneous administration or oral administration.

23. A method of determining malignancy or invasiness of a tumor, comprising:
detecting at least one or more Eph biomarkers and/or ligands thereof in a subject sample, and; correlating the detection of one or more Eph biomarkers and/or ligands thereof, with a diagnosis of a malignant tumor, wherein the correlation takes into account the detection of one or more biomarker in each diagnosis, as compared to normal subjects wherein the Eph biomarkers are one or more of: EphA1, EphA2, EphA3, EphA4, EphA5, EphA6, EphA7, EphA8, EphB1, EphB2, EphB3, EphB4 and EphB5, eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk and nuk receptors, peptides or fragments thereof, and; the ligands comprising at least one of: ephrin A1, ephrin-A2, ephrin-A3, ephrin-A4, ephrin-A5, ephrin-B1, ephrin-B2 or ephrin-B3, variants and fragments thereof,
correlating the detection of one or more biomarkers with a diagnosis of malignancy, wherein the correlation takes into account the detection of one or more biomarkers in each diagnosis, as compared to normal subjects.

24. The method of claim 23, wherein the Eph biomarker is inactive EphA2 and is detected in increased levels as compared to benign tumors and normal cells.

25. A kit for diagnosing cancer in a subject, the kit comprising:
at least one biomarker identified by any one of EphA1, EphA2, EphA3, EphA4, EphA5, EphA6, EphA7, EphA8, EphB1, EphB2, EphB3, EphB4 and EphB5, eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk, nuk, ephrin A1, ephrin-A2, ephrin-A3, ephrin-A4, ephrin-A5, ephrin-B1, ephrin-B2 or ephrin-B3;
a substrate for holding a biological sample isolated from a human subject suspected of having a damaged nerve cell,
an antibody that detects at least one or more of the biomarkers; and,
printed instructions for reacting the agent with the biological sample or a portion of the biological sample to detect the presence or amount of at least one marker in the biological sample.

26. The kit of claim 25, wherein the kit comprises a plurality of biomarkers as identified by EphA1, EphA2, EphA3, EphA4, EphA5, EphA6, EphA7, EphA8, EphB1, EphB2, EphB3, EphB4 and EphB5, eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk, nuk, ephrin A1, ephrin-A2, ephrin-A3, ephrin-A4, ephrin-A5, ephrin-B1, ephrin-B2 or ephrin-B3.

27. The kit of claim 25, wherein the kit comprises at least one antibody that is specific for any one of: EphA1, EphA2, EphA3, EphA4, EphA5, EphA6, EphA7, EphA8, EphB1, EphB2, EphB3, EphB4 and EphB5, eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk, nuk, ephrin A1, ephrin-A2, ephrin-A3, ephrin-A4, ephrin-A5, ephrin-B1, ephrin-B2 or ephrin-B3.

28. A method of identifying candidate therapeutic agents for treatment of tumors, comprising:
culturing an isolated cell expressing a receptor comprising any one of: ephrin A1, ephrin-A2, ephrin-A3, ephrin-A4, ephrin-A5, ephrin-B1, ephrin-B2 or ephrin-B3, variants and fragments thereof,
administering a candidate therapeutic agent to the cultured cell; and,
correlating expression levels and phosphorylation of the receptor in the presence or absence of a candidate therapeutic agent as compared to a normal cell and a cell cultured in the presence of an ephrin molecule; and,
identifying candidate therapeutic agents that bind to ephrin molecules, thereby,
identifying candidate therapeutic agents for treatment of tumors.

29. The method of claim 28, wherein candidate therapeutic agents are administered to normal cells and cancer cells and assayed for binding to ephrin molecules.

30. The method of claim 28, wherein binding of the candidate therapeutic agents to ephrin molecules is compared to binding of said agents to normal cells and cancer cells.

31. The method of claim 28, wherein binding of the candidate therapeutic agents to ephrin molecules is compared to expression and activation of Eph molecules, wherein activation of Eph molecules is determined by phosphorylation of tyrosine molecules as compared to Eph on a normal cell, Eph in the presence of its ephrin ligand and Eph in the presence of a candidate therapeutic agent.

32. The method of claim 31, wherein expression of Eph is compared to Eph in a normal cell, Eph+ cells in the presence of its ephrin ligand and Eph+ cells in the presence of a candidate therapeutic agent.
